SEQUENCE LISTING (33 sequences)

SEQ ID NO: 1 - DNA, 30 bp, synthetic construct
  source 1..30: mol_type = other DNA
agcaatacgtaactgaacgaagtaccctaa

SEQ ID NO: 2 - DNA, 30 bp, synthetic construct
  source 1..30: mol_type = other DNA
agcaatacgtaactgaacgaagtaacccta

SEQ ID NO: 3 - DNA, 30 bp, synthetic construct
  source 1..30: mol_type = other DNA
agcaatacgtaactgaacgaagttaaccct

SEQ ID NO: 4 - DNA, 30 bp, synthetic construct
  source 1..30: mol_type = other DNA
agcaatacgtaactgaacgaagtctaaccc

SEQ ID NO: 5 - DNA, 30 bp, synthetic construct
  source 1..30: mol_type = other DNA
agcaatacgtaactgaacgaagtcctaacc

SEQ ID NO: 6 - DNA, 30 bp, synthetic construct
  source 1..30: mol_type = other DNA
agcaatacgtaactgaacgaagtccctaac

SEQ ID NO: 7 - DNA, 23 bp, synthetic construct
  source 1..23: mol_type = other DNA
agcaatacgtaactgaacgaagt

SEQ ID NO: 8 - DNA, 23 bp, synthetic construct
  source 1..23: mol_type = other DNA
acttcgttcagttacgtattgct

SEQ ID NO: 9 - DNA, 29 bp, synthetic construct
  source 1..29: mol_type = other DNA
acttcgttcagttacgtattgctagcaat

SEQ ID NO: 10 - DNA, 24 bp, synthetic construct
  source 1..24: mol_type = other DNA
acttcgttcagttacgtattgcta

SEQ ID NO: 11 - DNA, 16 bp, synthetic construct
  source 1..16: mol_type = other DNA
gcttgggtgtttaacc

SEQ ID NO: 12 - DNA, 41 bp, synthetic construct
  source 1..41: mol_type = other DNA
agcaatacgtaactgaacgaagtccctaaccctaaccctaa

SEQ ID NO: 13 - DNA, 41 bp, synthetic construct
  source 1..41: mol_type = other DNA
agcaatacgtaactgaacgaagtctaaccctaaccctaacc

SEQ ID NO: 14 - DNA, 41 bp, synthetic construct; identical to SEQ ID NO: 12
  source 1..41: mol_type = other DNA

SEQ ID NO: 15 - DNA, 41 bp, synthetic construct
  source 1..41: mol_type = other DNA
agcaatacgtaactgaacgaagttaaccctaaccctaaccc

SEQ ID NO: 16 - DNA, 41 bp, synthetic construct; identical to SEQ ID NO: 13
  source 1..41: mol_type = other DNA

SEQ ID NO: 17 - DNA, 41 bp, synthetic construct
  source 1..41: mol_type = other DNA
agcaatacgtaactgaacgaagtcctaaccctaaccctaac

SEQ ID NO: 18 - DNA, 41 bp, synthetic construct
  source 1..41: mol_type = other DNA
agcaatacgtaactgaacgaagtaaccctaaccctaaccct

SEQ ID NO: 19 - DNA, 41 bp, synthetic construct
  source 1..41: mol_type = other DNA
agcaatacgtaactgaacgaagtaccctaaccctaacccta

SEQ ID NO: 20 - DNA, 29 bp, synthetic construct
  source 1..29: mol_type = other DNA
acttcgttcggtgacttgaggacagcaat

SEQ ID NO: 21 - DNA, 48 bp, synthetic construct
  source 1..48: mol_type = other DNA
ttagggttagggttagggttagggttagggttagggttagggttaggg

SEQ ID NO: 22 - DNA, 18 bp, synthetic construct
  source 1..18: mol_type = other DNA
ccctaaccctaaccctaa

SEQ ID NO: 23 - DNA, 48 bp, synthetic construct
  source 1..48: mol_type = other DNA
agcaatacgtaactgaacgaagtaccctaaccctaaccctaaccctaa

SEQ ID NO: 24 - DNA, 54 bp, synthetic construct
  source 1..54: mol_type = other DNA
attgctagcaatacgtaactgaacgaagtaccctaaccctaaccctaaccctaa

SEQ ID NO: 25 - DNA, 50 bp, synthetic construct
  source 1..50: mol_type = other DNA
  modified_base 17..20: mod_base = OTHER; note = 5-Nitroindole
gcttgggtgtttaaccnnnnagcaatacgtaactgaacgaagtaccctaa

SEQ ID NO: 26 - DNA, 50 bp, synthetic construct
  source 1..50: mol_type = other DNA
  modified_base 17..20: mod_base = OTHER; note = 5-Nitroindole
gcttgggtgtttaaccnnnnagcaatacgtaactgaacgaagtaacccta

SEQ ID NO: 27 - DNA, 50 bp, synthetic construct
  source 1..50: mol_type = other DNA
  modified_base 17..20: mod_base = OTHER; note = 5-Nitroindole
gcttgggtgtttaaccnnnnagcaatacgtaactgaacgaagttaaccct

SEQ ID NO: 28 - DNA, 50 bp, synthetic construct
  source 1..50: mol_type = other DNA
  modified_base 17..20: mod_base = OTHER; note = 5-Nitroindole
gcttgggtgtttaaccnnnnagcaatacgtaactgaacgaagtctaaccc

SEQ ID NO: 29 - DNA, 50 bp, synthetic construct
  source 1..50: mol_type = other DNA
  modified_base 17..20: mod_base = OTHER; note = 5-Nitroindole
gcttgggtgtttaaccnnnnagcaatacgtaactgaacgaagtcctaacc

SEQ ID NO: 30 - DNA, 50 bp, synthetic construct
  source 1..50: mol_type = other DNA
  modified_base 17..20: mod_base = OTHER; note = 5-Nitroindole
gcttgggtgtttaaccnnnnagcaatacgtaactgaacgaagtccctaac

SEQ ID NO: 31 - DNA, 68 bp, synthetic construct
  source 1..68: mol_type = other DNA
  modified_base 17..20: mod_base = OTHER; note = 5-Nitroindole
gcttgggtgtttaaccnnnnagcaatacgtaactgaacgaagtaccctaaccctaaccctaaccctaa

SEQ ID NO: 32 - DNA, 63 bp, synthetic construct
  source 1..63: mol_type = other DNA
tnnnnnnnnnnnnnttagggttagggttagggttagggttagggttagggttagggttaggga

SEQ ID NO: 33 - DNA, 63 bp, synthetic construct
  source 1..63: mol_type = other DNA
tccctaaccctaaccctaaccctaaccctaaccctaaccctaaccctaannnnnnnnnnnnna

(19) United States
(12) Patent Application Publication
(10) Pub. No.: US 2025/0361552 A1
(43) Pub. Date: Nov. 27, 2025

(54) METHOD AND ADAPTORS

(71) Applicant: Oxford Nanopore Technologies PLC, Oxford (GB)

(72) Inventors: Daniel George Fordham, Oxford (GB); Xiaguang Dai, Oxford (GB); Scott Hickey, Oxford (GB); Preeyesh Rughani, Oxford (GB); Carly Tyler, Oxford (GB)

(73) Assignee: Oxford Nanopore Technologies PLC, Oxford (GB)

(21) Appl. No.: 18/866,113

(22) PCT Filed: May 16, 2023
(86) PCT No.: PCT/EP2023/063061

§ 371 Date: Nov. 15, 2024

Related U.S. Application Data
(60) Provisional application No. 63/342,802, filed on May 17, 2022.

(51) Int. Cl. C12Q 1/6855

(52) U.S. Cl. CPC C12Q 1/6855

(57) ABSTRACT

The invention relates to methods for characterising, such as sequencing, at least part of a telomere and adaptors for use in such methods.

### RELATED APPLICATIONS

The present invention is a national stage filing under 35 U.S.C. § 371 of international PCT application PCT/EP2023/063061, filed May 16, 2023, which claims priority under 35 U.S.C. § 119(e) to U.S. provisional patent application, U.S. Ser. No. 63/342,802, filed May 17, 2022, the entire contents of each of which are herein incorporated by reference.

### REFERENCE TO AN ELECTRONIC SEQUENCE LISTING

The contents of the electronic sequence listing (0036670128US01-SEQ-KZM.xml; Size: 34,282 bytes; and Date of Creation: Nov. 12, 2024) is herein incorporated by reference in its entirety.

### TECHNICAL FIELD

The invention relates to methods for characterising, such as sequencing, at least part of a telomere and adaptors for use in such methods.

### BACKGROUND

Telomeres, the regions of repetitive DNA sequences at the end of eukaryotic chromosomes, are difficult to characterise, such as sequence. This is for a variety of reasons including, but not limited to, them being highly repetitive and non-linear, looping back on themselves to result in a displacement loop (D-loop), and telomere-binding proteins binding along the D-loop. Methods of sequencing telomeres are known but these typically involve restriction digestion and/or PCR and Southern blot analysis (Lai, T P., Zhang, N., Noh, J. et al. Nat Commun 8, 1356 (2017); Bendix L, Horn P B, Jensen U B, Rubelj I, Kolvraa S. Aging Cell. 2010 June; 9(3):383-97; and Bennett H W, Liu N, Hu Y, King M C. FEBS Lett. 2016 December; 590(23):4159-4170). The sequence of the telomere has to be reconstructed from shorter sequences in these methods, and they focus on characterising short telomeres. The use of PCR is problematic because polymerases cannot effectively copy long stretches of repetitive sequences as found in telomeres.

Biological pores (and other nanopores) have great potential as direct, electrical biosensors for polymers and a variety of small molecules. In particular, recent focus has been given to nanopores as a potential DNA sequencing technology. When a potential is applied across a nanopore, there is a change in the current flow when an analyte, such as a nucleotide, resides transiently in the barrel for a certain period of time. Nanopore detection of the nucleotide gives a current change of known signature and duration. In the strand sequencing method, a single polynucleotide strand is passed through the pore and the identities of the nucleotides are derived. Strand sequencing can involve the use of a molecular brake to control the movement of the polynucleotide through the pore.

Nanopores have been used to characterise telomeres by extending the 3′ overhang of the telomere with a polyA tail (Sholes S L, Karimian K, Gershman A, Kelly T J, Timp W, Greider C W. Genome Res. 2022 April; 32(4):616-628. doi: 10.1101/gr.275868.121.), but this method was not specific to telomeres and many other 3′ overhangs in genomic DNA were extended. The method also used polymerases to fill-in overhangs before additional tagging. There is a need for improved methods of characterising telomeres.

### SUMMARY OF THE INVENTION

The present inventors have identified a specific method for characterising, such as sequencing, at least part of a telomere. The method involves ligating a polynucleotide telomere adaptor to the 5′ end of the non-overhanging strand at the end of the telomere. As described in more detail below, the 3′ end of the adaptor specifically hybridises to the first part of the overhanging strand and so only the end of the telomere will be adapted. The telomere adaptor can then be used to characterise the ligated, non-overhanging strand of the at least part of the telomere in the in the 5′ to 3′ direction from the end of the telomere. The telomere adaptor is not ligated to any other part of the chromosome and so the method specifically characterises the at least part of the telomere. In essence, the method is an enrichment strategy to specifically characterise the at least part of the telomere. Part or all of the telomere may be specifically characterised and the method may also involve characterising part or all of one or more of the subtelomere, the chromatin (i.e., genomic DNA), the opposite subtelomere and the opposite telomere. The method may be used in combination with nanopore sequencing but does not have to be. The method of the invention has several advantages which include, but are not limited to, specific and effective characterisation of the at least part of the telomere, high resolution of sequence as well as the length, the ability to characterise beyond the telomere and further into the chromosome, including the possibility of whole chromosome characterisation, no requirement for restriction digestion and fragment size control, no requirement for PCR, the ability to identify methylated nucleotides in the telomere or other modifications, the ability to identify the presence or absence of telomere binding proteins and to characterise such proteins.

In a preferred embodiment, the method also comprises creating a double stranded break at the opposite end of the at the opposite end of the at least part of the telomere from the telomere end and characterising the non-ligated, overhanging strand of the at least part of the telomere in the opposite direction, i.e., towards the end of the telomere. This allows both strands of the at least part of the telomere to be characterised and provides increased resolution, especially with regards to sequencing of the repetitive telomeric sequence and identifying modifications, such as methylation.

The invention provides a method for characterising at least part of a telomere, the method comprising:
(a) ligating a polynucleotide telomere adaptor to the 5′ end of the non-overhanging strand at the end of the telomere wherein the 3′ end of the adaptor specifically hybridises to the first part of the overhanging strand and the 5′ end of the adaptor does not hybridise to the opposite part of the overhanging strand; and
(b) using the telomere adaptor to characterise the ligated, non-overhanging strand of the at least part of the telomere in the 5′ to 3′ direction from the end of the telomere.

The invention also provides a method for characterising at least part of a telomere, the method comprising (a) ligating a polynucleotide telomere adaptor to the 5′ end of the non-overhanging strand at the end of the telomere wherein the 3′ end of the specifically hybridises to the first part of the overhanging strand and the 5′ end of the adaptor does not hybridise to the opposite part of the overhanging strand, (b) using a polymer-guided effector protein to create a double stranded break at the opposite end of the at least part of the telomere from the telomere end and attaching a sequencing adaptor to the opposite end and (c) using the telomere adaptor to characterise the ligated non-overhanging strand of at least part of the telomere in the 5′ to 3′ direction from the end of the telomere and using the sequencing adaptor to characterise the non-ligated overhanging strand of the at least part of the telomere in the 5′ to 3′ direction to the end of the telomere.

The invention also provides:
a polynucleotide telomere adaptor wherein the 3′ end of the adaptor specifically hybridises to the first part of the overhanging strand at the end of a telomere and the 5′ end of the adaptor does not hybridise to the opposite part of the overhanging strand at the end of the telomere;
a population of six telomere adaptors each of which has a 3′ end which specifically hybridises to one of the six possible sequences of the first part of the overhanging strand at the end of a telomere and a 5′ end which does not hybridise to the opposite part of the overhanging strand at the end of the telomere.
a kit for characterising at least part of a telomere, comprising (a) one or more polynucleotide telomere adaptors of the invention or a population of six telomere adaptors of the invention and (b) one or more splint polynucleotides or one or more polynucleotide extensions; and
a system comprising (a) one or more polynucleotide telomere adaptors of the invention or a population of six telomere adaptors of the invention and (b) a nanopore.

### DESCRIPTION OF THE FIGURES

It is to be understood that Figures are for the purpose of illustrating particular embodiments of the invention only and are not intended to be limiting.

FIG. 1 shows T1 to T6 telomere adaptors as used in Example 1. Telomere adaptors are ligated to the C-rich strands at chromosome ends.

FIG. 2 shows annealing of telomere splint as per Example 1. T1=telomere adapter; S1=telomere splint variation; S2=telomere splint variation.

FIG. 3 shows ligation of a nanopore sequencing adapter (Oxford Nanopore Technologies). The sequencing adapter is ligated to the telomere adaptor T1. The ligation reaction is facilitated by the annealed splint S1.

FIG. 4 shows ligation of example “click” chemistry telomere adaptors. T1 to T6 represent different adaptors. The “click” telomere adaptors are ligated to the C-rich strands at chromosome ends.

FIG. 5 shows the addition of a “click” nanopore sequencing adapter (Oxford Nanopore Technologies) using “click” chemistry.

FIG. 6 shows annealing of biotinylated telomere adaptors. The biotinylated telomere adaptors are ligated to the C-rich strands at chromosome ends.

FIG. 7 shows ligation of nanopore sequencing adaptors (Oxford Nanopore Technologies). Sequencing adaptors were added following dA-tailing of both ends of the double-stranded DNA molecule.

FIG. 8 shows the mappable telomeric read counts per chromosome for a given method. 1× Teloseq begins the protocol as described in the Examples with 5 μg of DNA input, whereas 3× Teloseq begins the protocol with 15 μg of input. The read count for 3× Teloseq is 3 times more than 1× Teloseq, as expected.

FIG. 9 shows the read coverage per chromosome for a given condition. The reads were filtered and assigned to a chromosome. When a read has multiple mapping alignments, a single alignment is chosen by selecting the longest read coverage, highest identity and overlapping the telomere and sub-telomeric regions. The coverage was normalized by dividing total number of telomeric reads for a given chromosome over the total number of telomeric reads by a given method. Coverage is relatively the same between WGS and the “Teloseq” method of the invention, while the restriction digest methods show biases toward certain chromosomes.

FIG. 10 shows the total number of telomeric reads for each best run per method that were identified using the noise cancelling repeat finder (dark blue). Shows the number of telomeric reads that where mapped to the terminal ends of chromosome (light blue). If a read did not map to the terminal, they were discarded from the analysis. WGS is the control. 1× Teloseq begins the protocol with 5 μg of DNA input, where 3× Teloseq begins the protocol with 15 μg of input.

FIG. 11 shows the percentage of telomeric reads that are mappable and unmappable. Teloseq has ˜10% of telomeric reads that are not mappable. Conversely, BamHI has ˜40% of telomeric reads are not mappable.

FIG. 12 shows the percentage of methylated CpG sites for a single flowcell of 3× Teloseq. The blue bar is the percentage of the methylated CpG site and the red lines show the total CpG sites along the aligned region. For example, chromosome 3Q has over 800 CPG site however less than 40% are methylated, whereas chromosome 11 Q has 200 CPG sites and over 80% are methylated.

FIG. 13 shows an example of methylated CpG (dark grey) sites on chromosome 11 from a single run of 3× Telomere. The telomeric reads were re-basecalled with Remora to call methylation.

FIG. 14 shows the “Teloseq” method of the invention enabled the identification of the greatest number of telomere sequences. Each triplet set of bars shows (reading left to right): Telomeric read found; Telomeric read classified; Anchored reads.

FIG. 15 shows a telomere bioinformatics workflow and telomere enrichment with HG002.

FIG. 16 shows haplotyped HG002 telomeric reads to chromosome 18.q and read counts.

FIG. 17 shows telomere lengths by chromosome and haplotype.

### DESCRIPTION OF THE SEQUENCE LISTING

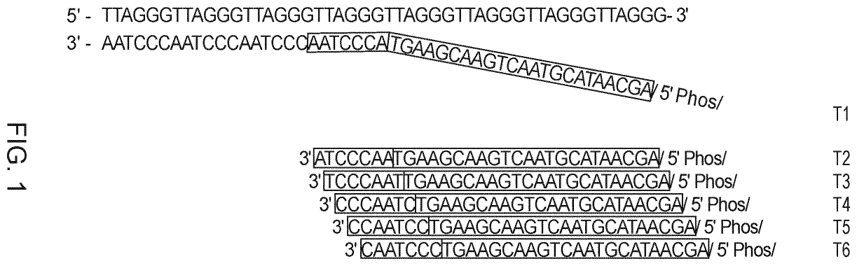

SEQ ID NOs: 1-6 show the sequences of the telomere adaptors used in Example 1 (Table 3 and FIG. 1).

SEQ ID NO: 7 shows a preferred 5′ end of a telomere adaptor of the invention. This is present in all of SEQ ID NOs: 1-6 and 14-19.

SEQ ID NO: 8 shows a preferred sequence for the splint polynucleotide and is the reverse complement of SEQ ID NO: 7.

SEQ ID NOs: 9-10 show the splint polynucleotides used in the Examples (Table 5).

SEQ ID NO: 11 shows the polynucleotide extension used in the Examples (Table 7).

SEQ ID NOs: 12-13 show the sequences of the biotinylated telomere adaptors used in the Examples (Table 9).

SEQ ID NOs: 14-19 show the sequences of the telomere adaptors used in Example 10 (Table 14).

SEQ ID NO: 20 shows the splint polynucleotide used in the Example 10 (Table 15).

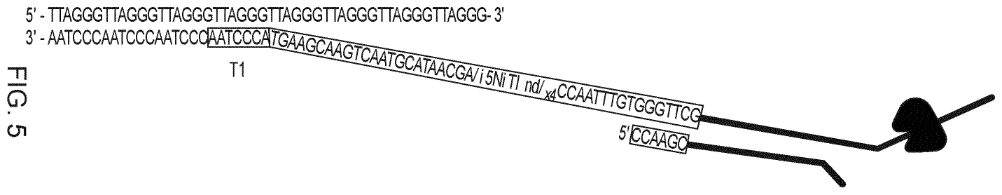

SEQ ID NO: 21 shows the sequence of the top (overhanging) strand of the exemplary chromosome end in FIGS. 1-6.

SEQ ID NO: 22 show the sequence (in the 5′ to 3′ direction) of the bottom (non-overhanging) strand of the exemplary chromosome end in FIGS. 1-6.

SEQ ID NO: 23 shows the sequence (in the 5′ to 3′ direction) formed by attachment of the T1 telomere adaptor to the non-overhanging strand in FIG. 1.

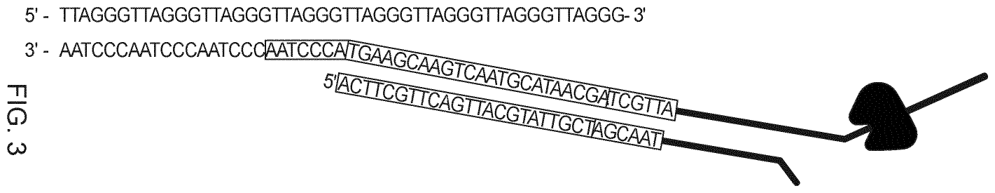

SEQ ID NO: 24 shows the sequence (in the 5′ to 3′ direction) formed by attachment of the sequencing adaptor to the telomere adaptor in FIG. 3. This sequence includes SEQ ID NO: 23.

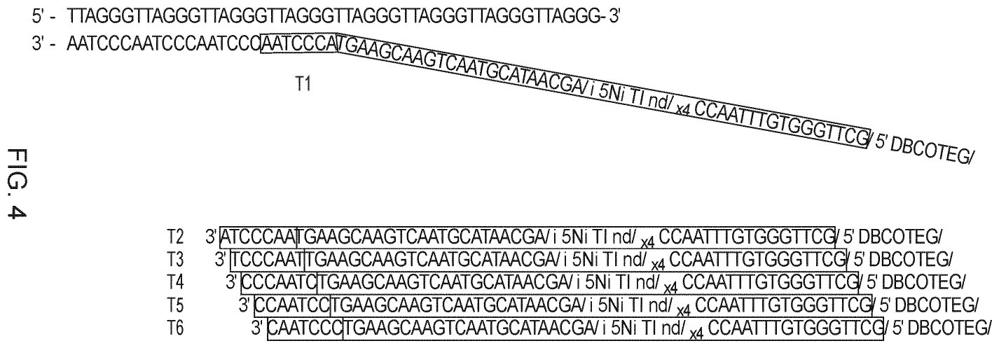

SEQ ID 25-30 show the sequences (in the 5′ to 3′ direction) of the extended telomere adaptors in FIG. 4.

SEQ ID NO: 31 shows the sequence (in the 5′ to 3′ direction) formed by attachment of the extended T1 telomere adaptor to the non-overhanging strand in FIG. 4.

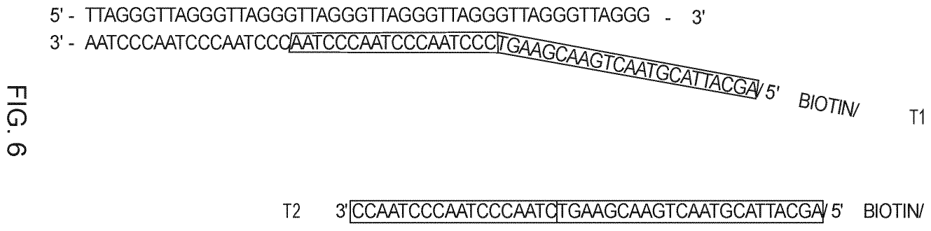

SEQ ID NO: 32 shows the sequence of the top strand in FIG. 6.

SEQ ID NO: 33 shows the sequence (in the 5′ to 3′ direction) of the bottom strand in FIG. 6.

### DETAILED DESCRIPTION

It is to be understood that different applications of the disclosed products and methods may be tailored to the specific needs in the art. It is also to be understood that the terminology used herein is for the purpose of describing particular embodiments of the invention only and is not intended to be limiting.

All publications, patents and patent applications cited herein, whether supra or infra, are hereby incorporated by reference in their entirety. All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### Definitions

Where an indefinite or definite article is used when referring to a singular noun e.g., “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the invention. Practitioners are particularly directed to Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016), for definitions and terms of the art. The definitions provided herein should not be construed to have a scope less than understood by a person of ordinary skill in the art.

“About” as used herein when referring to a measurable value such as an amount, a temporal duration, and the like, is meant to encompass variations of ±20% or +10%, more preferably ±5%, even more preferably ±1%, and still more preferably ±0.1% from the specified value, as such variations are appropriate to perform the disclosed methods.

“Nucleotide sequence”, “DNA sequence” or “nucleic acid molecule(s)” as used herein refers to a polymeric form of nucleotides of any length, either ribonucleotides or deoxyribonucleotides. This term refers only to the primary structure of the molecule. Thus, this term includes double- and single-stranded DNA, and RNA. The term “nucleic acid” as used herein, is a single or double stranded covalently linked sequence of nucleotides in which the 3′ and 5′ ends on each nucleotide are joined by phosphodiester bonds. The polynucleotide may be made up of deoxyribonucleotide bases or ribonucleotide bases.

Nucleic acids may be manufactured synthetically in vitro or isolated from natural sources. Nucleic acids may further include modified DNA or RNA, for example DNA or RNA that has been methylated, or RNA that has been subject to post-translational modification, for example 5′-capping with 7-methylguanosine, 3′-processing such as cleavage and polyadenylation, and splicing. Nucleic acids may also include synthetic nucleic acids (XNA), such as hexitol nucleic acid (HNA), cyclohexene nucleic acid (CeNA), threose nucleic acid (TNA), glycerol nucleic acid (GNA), locked nucleic acid (LNA) and peptide nucleic acid (PNA). Sizes of nucleic acids, also referred to herein as “polynucleotides” are typically expressed as the number of base pairs (bp) for double stranded polynucleotides, or in the case of single stranded polynucleotides as the number of nucleotides (nt). One thousand bp or nt equal a kilobase (kb). Polynucleotides of less than around 40 nucleotides in length are typically called “oligonucleotides” and may comprise primers for use in manipulation of DNA such as via polymerase chain reaction (PCR).

The term “amino acid” in the context of the present disclosure is used in its broadest sense and is meant to include organic compounds containing amine (NH₂) and carboxyl (COOH) functional groups, along with a side chain (e.g., a R group) specific to each amino acid. The amino acids typically refer to naturally occurring L α-amino acids or residues. The commonly used one and three letter abbreviations for naturally occurring amino acids are used herein: A=Ala; C=Cys; D=Asp; E=Glu; F=Phe; G=Gly; H=His; I=Ile; K=Lys; L=Leu; M=Met; N=Asn; P=Pro; Q=Gln; R=Arg; S=Ser; T=Thr; V=Val; W=Trp; and Y=Tyr (Lehninger, A. L., (1975) Biochemistry, 2d ed., pp. 71-92, Worth Publishers, New York). The general term “amino acid” further includes D-amino acids, retro-inverso amino acids as well as chemically modified amino acids such as amino acid analogues, naturally occurring amino acids that are not usually incorporated into proteins such as norleucine, and chemically synthesised compounds having properties known in the art to be characteristic of an amino acid, such as β-amino acids. For example, analogues or mimetics of phenylalanine or proline, which allow the same conformational restriction of the peptide compounds as do natural Phe or Pro, are included within the definition of amino acid. Such analogues and mimetics are referred to herein as “functional equivalents” of the respective amino acid. Other examples of amino acids are listed by Roberts and Vellaccio, The Peptides: Analysis, Synthesis, Biology, Gross and Meiehofer, eds., Vol. 5 p. 341, Academic Press, Inc., N.Y. 1983, which is incorporated herein by reference.

The terms “polypeptide”, and “peptide” are interchangeably used herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally occurring amino acid polymers. Polypeptides can also undergo maturation or post-translational modification processes that may include, but are not limited to glycosylation, proteolytic cleavage, lipidization, signal peptide cleavage, propeptide cleavage, phosphorylation, and such like. A peptide can be made using recombinant techniques, e.g., through the expression of a recombinant or synthetic polynucleotide. A recombinantly produced peptide it typically substantially free of culture medium, e.g., culture medium represents less than about 20%, more preferably less than about 10%, and most preferably less than about 5% of the volume of the protein preparation.

The term “protein” is used to describe a folded polypeptide having a secondary or tertiary structure. The protein may be composed of a single polypeptide or may comprise multiple polypeptides that are assembled to form a multimer. The multimer may be a homooligomer, or a heterooligmer. The protein may be a naturally occurring, or wild type protein, or a modified, or non-naturally, occurring protein. The protein may, for example, differ from a wild type protein by the addition, substitution, or deletion of one or more amino acids.

A “variant” of a protein encompasses peptides, oligopeptides, polypeptides, proteins, and enzymes having amino acid substitutions, deletions and/or insertions relative to the unmodified or wild-type protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

For all aspects and embodiments of the invention, a “variant” has at least 50%, 60%, 70%, 80%, 90%, 95% or 99% complete sequence identity to the amino acid sequence of the corresponding wild-type protein. Sequence identity can also be to a fragment or portion of the full-length polynucleotide or polypeptide. Hence, a sequence may have only 50% overall sequence identity with a full-length reference sequence, but a sequence of a particular region, domain or subunit could share 80%, 90%, or as much as 99% sequence identity with the reference sequence.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified”, “mutant” or “variant” refers to a gene or gene product that displays modifications in sequence (e.g., substitutions, truncations, or insertions), post-translational modifications and/or functional properties (e.g., altered characteristics) when compared to the wild-type gene or gene product. It is noted that naturally occurring mutants can be isolated; these are identified by the fact that they have altered characteristics when compared to the wild-type gene or gene product. Methods for introducing or substituting naturally occurring amino acids are well known in the art. For instance, methionine (M) may be substituted with arginine (R) by replacing the codon for methionine (ATG) with a codon for arginine (CGT) at the relevant position in a polynucleotide encoding the mutant monomer. Methods for introducing or substituting non-naturally occurring amino acids are also well known in the art. For instance, non-naturally occurring amino acids may be introduced by including synthetic aminoacyl-tRNAs in the IVTT system used to express the mutant monomer. Alternatively, they may be introduced by expressing the mutant monomer in E. coli that are auxotrophic for specific amino acids in the presence of synthetic (i.e., non-naturally occurring) analogues of those specific amino acids. They may also be produced by naked ligation if the mutant monomer is produced using partial peptide synthesis. Conservative substitutions replace amino acids with other amino acids of similar chemical structure, similar chemical properties, or similar side-chain volume. The amino acids introduced may have similar polarity, hydrophilicity, hydrophobicity, basicity, acidity, neutrality, or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 1 below. Where amino acids have similar polarity, this can also be determined by reference to the hydropathy scale for amino acid side chains in Table 2.

TABLE 1
Chemical properties of amino acids

| | | | |
|---|---|---|---|
| Ala | aliphatic, hydrophobic, | Met | hydrophobic, neutral |
| | neutral | | |
| Cys | polar, hydrophobic, neutral | Asn | polar, hydrophilic, neutral |
| Asp | polar, hydrophilic, charged | Pro | hydrophobic, neutral |
| | (−) | | |
| Glu | polar, hydrophilic, charged | Gln | polar, hydrophilic, neutral |
| | (−) | | |
| Phe | aromatic, hydrophobic, | Arg | polar, hydrophilic, charged |
| | neutral | | (+) |
| Gly | aliphatic, neutral | Ser | polar, hydrophilic, neutral |
| His | aromatic, polar, | Thr | polar, hydrophilic, neutral |
| | hydrophilic, charged (+) | | |
| Ile | aliphatic, hydrophobic, | Val | aliphatic, hydrophobic, |
| | neutral | | neutral |
| Lys | polar, hydrophilic, | Trp | aromatic, hydrophobic, |
| | charged(+) | | neutral |
| Leu | aliphatic, hydrophobic, | Tyr | aromatic, polar, |
| | neutral | | hydrophobic |

TABLE 2
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

A mutant or modified protein, monomer or peptide can also be chemically modified in any way and at any site. A mutant or modified monomer or peptide is preferably chemically modified by attachment of a molecule to one or more cysteines (cysteine linkage), attachment of a molecule to one or more lysines, attachment of a molecule to one or more non-natural amino acids, enzyme modification of an epitope or modification of a terminus. Suitable methods for carrying out such modifications are well-known in the art. The mutant of modified protein, monomer or peptide may be chemically modified by the attachment of any molecule. For instance, the mutant of modified protein, monomer or peptide may be chemically modified by attachment of a dye or a fluorophore.

### METHODS OF THE INVENTION

The invention provides a method for characterising at least part of a telomere. Telomeres are regions of repetitive DNA sequences at the ends of a eukaryotic chromosome. Each chromosome has a telomere at each end. Telomeres protect the terminal regions of chromosomal DNA from progressive degradation and ensure the integrity of linear chromosomes by preventing DNA repair systems from mistaking the very ends of the DNA strand for a double strand break. Chromosomes are structures formed from long DNA molecules containing all or part of the genetic material in a eukaryotic cell. Sections of DNA called subtelomeres typically separate telomeres from the chromatin (i.e., genomic DNA) in chromosomes. The structure of a chromosome is typically telomere-subtelomere-chromatin-subtelomere-telomere.

The term “part” in the at least part of a telomere is interchangeable with “portion”. The part may be any amount of the telomere. The at least part of the telomere is preferably at least about 5%, such as at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98% or at least about 99%, of the telomere. The method is preferably for characterising all of the telomere (or the whole telomere).

The method is preferably for characterising all of the telomere (or the whole telomere) and an additional part of the chromosome. The additional part of the chromosome may be any additional amount of the chromosome. The additional part may be any of the % s of rest of the chromosome discussed above with reference to the at least part of the telomere.

The method is preferably for characterising (i) all of the telomere, (ii) all of the telomere and at least part of, or all of, the subtelomere, (iii) all of the telomere, all of the subtelomere and at least part of, or all of, the chromatin, (iv) all of the telomere, all of the subtelomere, all of the chromatin and at least part of, or all of, the opposite subtelomere, or (v) all of the telomere, all of the subtelomere, all of the chromatin, all of the opposite subtelomere and at least part of, or all of, the opposite telomere. Chromatin is interchangeable with genomic DNA.

The method is preferably for characterising (i), i.e., all of the telomere. The method is preferably for characterising (ii). The method is preferably for characterising all of the telomere and all of the subtelomere. The method is preferably for characterising (iii). The method is preferably for characterising all of the telomere, all of the subtelomere and all of the chromatin. The method is preferably for characterising (iv). The method is preferably for characterising all of the telomere, all of the subtelomere, all of the chromatin and all of the opposite subtelomere. The method is preferably for characterising (v). The method is preferably for characterising all of the telomere, all of the subtelomere, all of the chromatin, all of the opposite subtelomere and all of the opposite telomere.

In any of (ii)-(v), at least part of the subtelomere/chromatin/opposite subtelomere/opposite telomere may be any of the % s discussed above with reference to the at least part of the telomere.

The method is preferably for characterising all of a chromosome (or a whole chromosome). The method of the invention is preferably repeated at the other end of a chromosome and the method comprises characterising both strands of the whole chromosome. The method of the invention preferably comprises conducting a method of the invention at both ends of a chromosome and comprises characterising both strands of the whole chromosome. Any of the method of the invention can be used at each end of the chromosome. The methods used at each end may be the same or different. The methods are preferably the same.

The method preferably comprises in step (b) characterising the ligated, non-overhanging strand of the (i) telomere, (ii) the at least part of, or all of, the subtelomere, (iii) the at least part of, or all of, the chromatin, (iv) the at least part of, or all of, the opposite subtelomere, or (v) the at least part of, or all of, the opposite telomere in the 5′ to 3′ direction from the end of the telomere. Any of the embodiments discussed above for (i)-(v) equally apply to step (b) of the method.

The at least part of telomere, including any of (i)-(v) discussed above, may be any length. For example, the at least part of a telomere may be at least about 10, at least about 50, at least about 100, at least about 150, at least about 200, at least about 250, at least about 300, at least about 400 or at least about 500 nucleotides or nucleotide pairs in length. The at least part of a telomere can be about 1000 or more nucleotides or nucleotide pairs, about 5000 or more nucleotides or nucleotide pairs in length, about 100000 or more nucleotides or nucleotide pairs in length, about 500,000 or more nucleotides or nucleotide pairs in length, about 1,000,000 or more nucleotides or nucleotide pairs in length, about 10,000,000 or more nucleotides or nucleotide pairs in length, about 100,000,000 or more nucleotides or nucleotide pairs in length, or about 200,000,000 or more nucleotides or nucleotide pairs in length, or the entire length of a chromosome.

The method is preferably for characterising at least part of one or both telomeres on each of two or more different chromosomes. This allows the characteristics of the telomeres on two or more different chromosomes to be compared. The two or more different chromosomes may be from the same cell, tissue, organism, or taxonomic rank (such as genus or species) or from different cells, tissues, organisms, or taxonomic ranks (such as genera or species). The cell(s), tissue(s), organism(s), or taxonomic rank(s) are typically eukaryotic. The method may be for characterising at least part of one or both telomeres on each of any number of two or more different chromosomes, such as about 3 or more, about 4 or more, about 5 or more, about 10 or more, about 15 or more, about 20 or more, about 25 or more, about 30 or more or about 40 or more different chromosomes. Preferred numbers of different chromosomes include, but are not limited to, 2, 4, 6, 7, 8, 9, 10, 11, 12, 14, 16, 17, 18, 20, 22, 24, 26, 28, 30, 31, 32, 34, 36, 38, 40, 42, 44, 46, 48, 50, 52, 54, 56, 58, 60, 62, 63, 64, 66, 68, 70, 72, 74, 76, 78, 80 and 82. The number of different chromosomes may be even higher in polyploidy cells, tissues, organisms or taxonomic ranks. The method is preferably for characterising at least part of one or both telomeres on each of 46 different chromosomes. In these embodiments, the method comprises (a) ligating a polynucleotide telomere adaptor to the 5′ end of the non-overhanging strand at the end of each telomere wherein the 3′ end of the adaptor specifically hybridises to the first part of the overhanging strand and the 5′ end of the adaptor does not hybridise to the opposite part of the overhanging strand and (b) using each telomere adaptor to characterise the ligated, non-overhanging strand of the at least part of each telomere in the 5′ to 3′ direction from the end of each telomere.

The method preferably comprises (a) sequencing the at least part of the telomere, (b) measuring the length of the at least part of the telomere, (c) telomere to telomere assembly of a chromosome or genome, (d) identifying telomere or chromosome fusions, (e) identifying one or more modifications in the at least part of the telomere, (f) identifying the at least part of the telomere as a variant or (g) linking the at least part of the telomere to a particular cell or tissue type. The method may comprise any number and combination of (a)-(g).

The method preferably comprises (a). Methods of sequencing the ligated, non-overhanging strand are discussed in more detail below.

The method preferably comprises (b). The lack of digestion in the method of the invention means the length of the at least part of the telomere or all of the telomere can easily be measured in one step.

The method preferably comprises (c). As described above, the method allows whole chromosomes to be characterised from telomere to telomere. Also as discussed above, the method may be applied to two or more different chromosomes meaning whole genomes may be characterised.

The method preferably comprises (d). By characterising the at least part of the telomere, it is possible using the method to identify telomere fusions.

The method preferably comprises (e). The one or more modifications preferably comprise one or more of (i) methylation of one or more nucleotides, (ii) oxidation of one or more nucleotides, and (iii) damage to one or more nucleotides. The method may identify (i), (ii), (iii), (i) and (ii), (i) and (iii), (ii) and (iii) or (i), (ii) and (iii). For instance, the at least part of the telomere may comprise one or more pyrimidine dimers. Such dimers are typically associated with damage by ultraviolet light and are the primary cause of skin melanomas. Nanopore sequencing is capable of identifying methylated, oxidised, and damaged nucleotides.

The method preferably comprises (f). The method may identify the at least part of the telomere as a variant comprising one or more of (i) a telomere deletion, (ii) a telomere addition, and/or (ii) a telomere substitution. The method may identify the at least part of the telomere as a variant comprising (i), (ii), (iii), (i) and (ii), (i) and (iii), (ii) and (iii) or (i), (ii) and (iii). (i) may comprise the deletion of any number of nucleotides from the telomere. (i) may comprise a single deletion or multiple deletions. The deletion(s) may be in one or both strands. (ii) may comprise the addition of any number of nucleotides to the telomere. (ii) may comprise a single addition or multiple additions. The addition(s) may be in one or both strands. (iii) may comprise the substitution of any number of nucleotides within the telomere. The substitution(s) may be in one or both strands.

The method preferably comprises (g). If the method is conducted in a specific cell, tissue, organism, or taxonomic rank (such as genus or species), the at least part of the telomere can be linked to the cell, tissue, organism, or taxonomic rank (such as genus or species). The method of the invention may be carried out on different chromosomes from different cells, tissues or taxonomic ranks as discussed above and so each at least part of the telomere may be linked to different cells, tissues, or taxonomic ranks. The cell(s), tissue(s), organism(s), or taxonomic rank(s) are typically eukaryotic.

### Telomere Adaptors

The method of the invention comprises in step (a) ligating a polynucleotide telomere adaptor to the 5′ end of the non-overhanging strand at the end of the telomere. Methods for ligating polynucleotides are known in the art. The telomere adaptor is formed from at least one polynucleotide as discussed below. Polynucleotide telomere adaptor is interchangeable with telomere adaptor.

Telomeres typically have a 3′ overhang (see FIG. 1). This means the strand in the 5′ to 3′ direction typically overhangs at the end of the telomere. The strand in the 3′ to 5′ direction at the end of the telomere is typically non-overhanging. “Non-overhanging” is interchangeable with “underhanging”. Both strands at the end of the telomere are typically DNA.

The telomere adaptor may be any type of polynucleotide. A polynucleotide, such as a nucleic acid, is a macromolecule comprising two or more nucleotides. A polynucleotide can be single-stranded or double-stranded. A double-stranded polynucleotide is made of two single stranded polynucleotides hybridised together. The polynucleotide telomere adaptor can be a single-stranded polynucleotide or a double-stranded polynucleotide.

A polynucleotide may comprise any combination of any nucleotides. The nucleotides can be naturally occurring or artificial. A nucleotide typically contains a nucleobase, a sugar and at least one phosphate group. The nucleobase and sugar form a nucleoside. The nucleobase is typically heterocyclic. Nucleobases include, but are not limited to, purines and pyrimidines and more specifically adenine (A), guanine (G), thymine (T), uracil (U) and cytosine (C).

The sugar is typically a pentose sugar. Nucleotide sugars include, but are not limited to, ribose and deoxyribose. The sugar is preferably a deoxyribose. The polynucleotide preferably comprises the following nucleosides: deoxyadenosine (dA), deoxyuridine (dU) and/or thymidine (dT), deoxyguanosine (dG) and deoxycytidine (dC).

The nucleotide is typically a ribonucleotide or deoxyribonucleotide. The nucleotide typically contains a monophosphate, diphosphate, or triphosphate. The nucleotide may comprise more than three phosphates, such as 4 or 5 phosphates. Phosphates may be attached on the 5′ or 3′ side of a nucleotide. Nucleotides include, but are not limited to, adenosine monophosphate (AMP), guanosine monophosphate (GMP), thymidine monophosphate (TMP), uridine monophosphate (UMP), 5-methylcytidine monophosphate, 5-hydroxymethylcytidine monophosphate, cytidine monophosphate (CMP), cyclic adenosine monophosphate (cAMP), cyclic guanosine monophosphate (cGMP), deoxyadenosine monophosphate (dAMP), deoxyguanosine monophosphate (dGMP), deoxythymidine monophosphate (dTMP), deoxyuridine monophosphate (dUMP), deoxycytidine monophosphate (dCMP) and deoxymethylcytidine monophosphate. The nucleotides are preferably selected from AMP, TMP, GMP, CMP, UMP, dAMP, dTMP, dGMP, dCMP and dUMP.

A nucleotide may be abasic (i.e., lack a nucleobase). A nucleotide may also lack a nucleobase and a sugar (i.e., is a C3 spacer).

The nucleotides in the polynucleotide may be attached to each other in any manner. The nucleotides are typically attached by their sugar and phosphate groups as in nucleic acids. The nucleotides may be connected via their nucleobases as in pyrimidine dimers.

The polynucleotide can be a nucleic acid, such as deoxyribonucleic acid (DNA) or ribonucleic acid (RNA). The polynucleotide can comprise one strand of RNA hybridized to one strand of DNA. The polynucleotide may be any synthetic nucleic acid known in the art, such as peptide nucleic acid (PNA), glycerol nucleic acid (GNA), threose nucleic acid (TNA), locked nucleic acid (LNA), bridged nucleic acid (BNA) or other synthetic polymers with nucleotide side chains. The PNA backbone is composed of repeating N-(2-aminoethyl)-glycine units linked by peptide bonds. The GNA backbone is composed of repeating glycol units linked by phosphodiester bonds. The TNA backbone is composed of repeating threose sugars linked together by phosphodiester bonds. LNA is formed from ribonucleotides as discussed above having an extra bridge connecting the 2′ oxygen and 4′ carbon in the ribose moiety.

The polynucleotide is preferably DNA, RNA or a DNA or RNA hybrid, most preferably DNA. A DNA/RNA hybrid may comprise DNA and RNA on the same strand. Preferably, the DNA/RNA hybrid comprises one DNA strand hybridized to a RNA strand.

The backbone of the polynucleotide can be altered to reduce the possibility of strand scission. For example, DNA is known to be more stable than RNA under many conditions. The backbone of the polynucleotide strand can be modified to avoid damage caused by e.g., harsh chemicals such as free radicals. DNA or RNA that contains unnatural or modified bases can be produced by amplifying natural DNA or RNA polynucleotides in the presence of modified NTPs using an appropriate polymerase.

The telomere adaptor can be any length. For example, the telomere adaptor can be at least about 10, at least about 15, at least about 20, at least about 25, at least about 30, at least about 40 or at least about 50 nucleotides or nucleotide pairs in length.

The telomere adaptor is typically a single-stranded oligonucleotide. Oligonucleotides are short nucleotide polymers which typically have about 50 or fewer nucleotides, such about 45 or fewer, about 42 or fewer, about 41 or fewer, about 40 or fewer, about 35 or fewer or about 30 or fewer nucleotides. The telomere adaptor is preferably from about 15 to about 50 nucleotides in length, such as from about 20 to about 45 nucleotides in length. For example, the oligonucleotide can be about 15, about 16, about 17, about 18, about 19, about 20, about 21, about 22, about 23, about 24, about 25, about 26, about 27, about 28, about 29, about 30 nucleotides, about 35 nucleotides, about 40 nucleotides, about 41 nucleotides, about 42 nucleotides or about 45 nucleotides in length. The polynucleotide telomere adaptor is most preferably about 30 nucleotides or about 41 nucleotides in length. The different regions of the telomere adaptor are discussed in more detail below.

The telomere adaptor is typically synthetic or semi-synthetic. For example, DNA or RNA may be purely synthetic, synthesised by conventional DNA synthesis methods such as phosphoramidite based chemistries. Synthetic polynucleotides subunits may be joined together by known means, such as ligation or chemical linkage, to produce longer strands. Internal self-forming structures (e.g., hairpins, quadruplexes) can be designed into the substrate, e.g., by ligating appropriate sequences. Synthetic polynucleotides can be copied and scaled up for production by means known in the art, including PCR, incorporation into bacterial factories, and the like.

The polynucleotide telomere adaptor has 5′ to 3′ directionality. The 3′ end of the adaptor specifically hybridises to the first part of the overhanging strand. The 3′ end may be any part or portion of the polynucleotide telomere adaptor, such as at least about 5%, at least about 10%, at least about 15%, at least about 20%, at least about 30%, at least about 40% or at least about 50%. The 3′ end of the adaptor may be any length, such as at least about 5 nucleotides, at least about 6 nucleotides, at least about 7 nucleotides, at least about 10 nucleotides or at least about 20 nucleotides in length.

The 3′ end of the adaptor is preferably about 18 or fewer nucleotides, such about 16 or fewer, about 15 or fewer about 14 or fewer, about 10 or fewer, about 8 or fewer or about 7 or fewer nucleotides. The 3′ end is preferably from about 4 to about 18 nucleotides in length, such as from about 5 to about 16, from about 6 to about 15 or from about 7 to about 10 nucleotides in length. For example, the 3′ end can be about 5, about 6, about 7, about 8, about 9, about 10, about 11, about 12, about 13, about 14, about 15, about 16, about 17 or about 18 nucleotides in length. The 3′ end of polynucleotide telomere adaptor is most preferably about 7 nucleotides in length or about 18 nucleotides in length.

The 3′ end of the telomere adaptor specifically hybridises to the first part of the overhanging strand. The first part of the overhanging strand may be any of the lengths discussed above with reference to the 3′ end. The first part of the overhanging strand is preferably the same length as the 3′ end of the telomere adaptor.

The 3′ end “specifically hybridises” to first part of the overhanging strand when it hybridises with preferential or high affinity to the first part of the overhanging strand but does not substantially hybridise, does not hybridise, or hybridises with only low affinity to other polynucleotide sequences, especially other sequences in the telomere or chromosome. Conditions that permit the hybridisation are well-known in the art (for example, Sambrook et al., 2001, Molecular Cloning: a laboratory manual, 3rd edition, Cold Spring Harbour Laboratory Press; and Current Protocols in Molecular Biology, Chapter 2, Ausubel et al., Eds., Greene Publishing and Wiley-Interscience, New York (1995)). Hybridisation can be carried out under low stringency conditions, for example in the presence of a buffered solution of 30 to 35% formamide, 1 M NaCl and 1% SDS (sodium dodecyl sulfate) at 37° C. followed by a 20 wash in from 1× (0.1650 M Na+) to 2× (0.33 M Na+) SSC (standard sodium citrate) at 50° C. Hybridisation can be carried out under moderate stringency conditions, for example in the presence of a buffer solution of 40 to 45% formamide, 1 M NaCl, and 1% SDS at 37° C., followed by a wash in from 0.5× (0.0825 M Na+) to 1× (0.1650 M Na+) SSC at 55° C. Hybridisation can be carried out under high stringency conditions, for example in the presence of a buffered solution of 50% formamide, 1 M NaCl, 1% SDS at 37° C., followed by a wash in 0.1× (0.0165 M Na+) SSC at 60° C. The 3′ end “specifically hybridises” if it hybridises to the first part of the overhanging strand with a melting temperature (Tm) that is at least 2° C., such as at least 3° C., at least 4° C., at least 5° C., at least 6° C., at least 7° C., at least 8° C., at least 9° C. or at least 10° C., greater than its Tm for other polynucleotide sequences. More preferably, the 3′ end hybridises to first part of the overhanging strand with a Tm that is at least 2° C., such as at least 3° C., at least 4° C., at least 5° C., at least 6° C., at least 7° C., at least 8° C., at least 9° C., at least 10° C., at least 20° C., at least 30° C. or at least 40° C., greater than its Tm for other polynucleotide sequences. Preferably, the 3′ end hybridises to the first part of the overhanging strand with a Tm that is at least 2° C., such as at least 3° C., at least 4° C., at least 5° C., at least 6° C., at least 7° C., at least 8° C., at least 9° C., at least 10° C., at least 20° C., at least 30° C. or at least 40° C., greater than its Tm for a polynucleotide which differs from the first part of the overhanging strand by one or more nucleotides, such as by 1, 2, 3, 4 or 5 or more nucleotides. The 3′ end typically hybridises to the first part of the overhanging strand with a Tm of at least 90° C., such as at least 92° C. or at least 95° C. Tm can be measured experimentally using known techniques, including the use of DNA microarrays, or can be calculated using publicly available Tm calculators, such as those available over the internet.

The 3′ end of the adaptor typically comprises or consists of a sequence at least about 80% identical to the reverse complement of sequence of the first part of the overhanging strand. The 3′ end of the adaptor preferably comprises or consists of a sequence at least about 85% identical or at least about 85.7% identical to the reverse complement of the sequence of the first part of the overhanging strand. The 3′ end of the adaptor more preferably comprises or consists of a sequence at least about 90%, at least about 95%, at least about 98% or at least about 99% identical to the reverse complement of the sequence of the first part of the overhanging strand. The 3′ end of the adaptor most preferably comprises or consists of a sequence which is the reverse complement of the sequence of the first part of the overhanging strand. By complementary, it is meant the 3′ end of the adaptor comprises or consists of a sequence 100% identical to the reverse complement of the sequence of the first part of the overhanging strand. Complementarity is typically determined using canonical Watson-Crick base pairing. In the most preferred embodiment, the 3′ end is single-stranded DNA and comprises or consists of a sequence which is the reverse complement of the sequence of the first part of the overhanging DNA strand at the end of the telomere.

Human telomeres typically comprise numerous repetitions of the sequence TTAGGG. The 3′ end of the telomere adaptor preferably comprises at least one sequence which specifically hybridises to or is the reverse complement of one of the six possible sequences of the first part of the overhanging strand. The six possible sequences in the 5′ to 3′ direction are typically TTAGGG, TAGGGT, AGGGTT, GGGTTA, GGTTAG and GTTAGG. The 3′ end of the telomere adaptor preferably comprises CCCTAA, ACCCTA, AACCCT, TAACCC, CTAACC or CCTAAC. The 3′ end of the telomere adaptor preferably comprises two or more consecutive repetitions of CCCTAA, ACCCTA, AACCCT, TAACCC, CTAACC or CCTAAC. The 3′ end of the telomere adaptor may comprise any number, such as 3 or more, 4 or more or 5 or more, consecutive repetitions of CCCTAA, ACCCTA, AACCCT, TAACCC, CTAACC or CCTAAC. The 3′ end preferably comprises or consists of three consecutive repetitions of CCCTAA, ACCCTA, AACCCT, TAACCC, CTAACC or CCTAAC.

The method may comprise the use of more than one polynucleotide telomere adaptor. Any number of telomere adaptors may be used, such as about 2 or more, about 3 or more, about 4 or more, about 5 or more, about 6 or more or about 10 or more. The method preferably comprises before step (a) contacting the telomere with a population of six telomere adaptors each of which has a 3′ end which specifically hybridises to or comprises the reverse complement of one of the six possible sequences of the first part of the overhanging strand and a 5′ end which does not hybridise to the opposite part of the overhanging strand. The population comprises sequences which specifically hybridise to or are the reverse complement of all of the six possible sequences. The six possible sequences in the 5′ to 3′ direction are typically TTAGGG, TAGGGT, AGGGTT, GGGTTA, GGTTAG and GTTAGG. The population of six telomere adaptors preferably each has a 3′ end which specifically hybridises to or is the reverse complement of one of the six possible sequences of the first part of the overhanging strand (i.e., one of CCCTAA, ACCCTA, AACCCT, TAACCC, CTAACC and CCTAAC). The population typically comprises adaptors comprising sequences which specifically hybridise to all six possible sequences or all six possible reverse complement sequences. The 3′ end of each adaptor may have two or more, such as such as 3 or more, 4 or more or 5 or more, consecutive repetitions of its one of the six possible reverse complement sequences (i.e., of CCCTAA, ACCCTA, AACCCT, TAACCC, CTAACC or CCTAAC). The 3′ end of each adaptor preferably has three consecutive repetitions of CCCTAA, ACCCTA, AACCCT, TAACCC, CTAACC or CCTAAC. All of the adaptors in the population may have multiple consecutive repetitions of its one of the six possible reverse complement sequences and preferably have the same number of repetitions.

The population preferably comprises six telomere adaptors, wherein the 3′ end of one adaptor comprises or consists of CCCTAA, the 3′ end of one adaptor comprises or consists of ACCCTA, the 3′ end of one adaptor comprises or consists of AACCCT, the 3′ end of one adaptor comprises or consists of TAACCC, the 3′ end of one adaptor comprises or consists of CTAACC, and the 3′ end of one adaptor comprises or consists of CCTAAC. The population preferably comprises six telomere adaptors, wherein the 3′ end of one adaptor comprises or consists of two or more, such as three, consecutive repetitions of CCCTAA, the 3′ end of one adaptor comprises or consists of two or more, such as three, consecutive repetitions of ACCCTA, the 3′ end of one adaptor comprises or consists of two or more, such as three, consecutive repetitions of AACCCT, the 3′ end of one adaptor comprises or consists of two or more, such as three, consecutive repetitions of TAACCC, the 3′ end of one adaptor comprises or consists of two or more, such as three, consecutive repetitions of CTAACC, and the 3′ end of one adaptor comprises or consists of two or more, such as three, consecutive repetitions of CCTAAC. The population preferably comprises six telomere adaptors, wherein the 3′ end of one adaptor comprises or consists of ACCCTAA, the 3′ end of one adaptor comprises or consists of AACCCTA, the 3′ end of one adaptor comprises or consists of TAACCCT, the 3′ end of one adaptor comprises or consists of CTAACCC, the 3′ end of one adaptor comprises or consists of CCTAACC, and the 3′ end of one adaptor comprises or consists of CCCTAAC. All of the specific sequences are given in the 5′ to 3′ direction.

The 5′ end(s) of the polynucleotide telomere adaptor(s) does/do not hybridise to the opposite part of the overhanging strand. This means the 5′ end(s) of the adaptor(s) does/do not form a double strand with the opposite part of the overhanging strand and is/are free for characterisation or modification as discussed in more detail below. Lack of hybridisation can be measured as discussed above. The 5′ end may be any part or portion of the polynucleotide telomere adaptor, such as at least about 50%, at least about 60%, at least 70%, at least about 80% or at least about 85%. The 5′ end of the adaptor may be any length, such as at least about 10 nucleotides, at least about 15 nucleotides, at least about 20 nucleotides, at least about 25 nucleotides or at least about 30 nucleotides in length.

The 5′ end of the adaptor is preferably 60 or fewer nucleotides, such about 50 or fewer, about 40 or fewer, about 30 or fewer or about 25 or fewer nucleotides. The 5′ end is preferably from about 15 to about 35 nucleotides in length, such as from about 18 to about 30 or from about 20 to 25 nucleotides in length. For example, the 5′ end can be about 18, about 19, about 20, about 21, about 22, about 23, about 24, about 25, about 26, about 27 or about 28 nucleotides in length. The 5′ end of polynucleotide telomere adaptor is most preferably about 23 nucleotides in length.

The skilled person is capable of designing a 5′ end that sufficiently mismatches the opposite part of the overhanging strand such that it does not hybridise. The 5′ end of the adaptor typically comprises or consists of a sequence less than about 20% identical to the reverse complement of sequence of the opposite part of the overhanging strand. The 5′ end of the adaptor preferably comprises or consists of a sequence which is less than about 15%, less than about 10%, less than about 5%, less than about 2%, or less than about 1% identical to the reverse complement of the sequence of the opposite part of the overhanging strand. The 5′ end of the adaptor most preferably comprises or consists of a sequence which 0% identical to (i.e., is non-complementary to) the reverse complement of the sequence of the opposite part of the overhanging strand. Complementarity or a lack thereof is typically determined using canonical Watson-Crick base pairing.

For human telomeres, the 5′ end does not comprise repetitions of CCCTAA. A preferred 5′ end comprises or consists of AGCAATACGTAACTGAACGAAGT (SEQ ID NO: 7). This is in the 5′ to 3′ direction. In embodiments where more than one polynucleotide telomere adaptors are used, the adaptors may have the same 5′ end or different 5′ ends. The nucleotide at 5′ end of the telomere adaptor is preferably modified, for instance with a phosphate group.

The telomere adaptor preferably comprises or consists of the sequence shown in any one of SEQ ID NOs: 1-6. These sequences are shown below, in Table 3 and in FIG. 1.

The method preferably comprises before step (a) contacting the telomere with a population of six telomere adaptors which comprise or consist of the following sequences:

(SEQ ID NO: 1)
AGCAATACGTAACTGAACGAAGTACCCTAA;
(SEQ ID NO: 2)
AGCAATACGTAACTGAACGAAGTAACCCTA;
(SEQ ID NO: 3)
AGCAATACGTAACTGAACGAAGTTAACCCT;
(SEQ ID NO: 4)
AGCAATACGTAACTGAACGAAGTCTAACCC;
(SEQ ID NO: 5)
AGCAATACGTAACTGAACGAAGTCCTAACC;
and
(SEQ ID NO: 6)
AGCAATACGTAACTGAACGAAGTCCCTAAC.

These are shown in Table 3 of Example 1 and FIG. 1.

The telomere adaptor preferably comprises or consists of the sequence shown in any one of SEQ ID NOs: 14-19. These sequences are shown below and in Table 14.

The method preferably comprises before step (a) contacting the telomere with a population of six telomere adaptors which comprise or consist of the following sequences:

(SEQ ID NO: 14)
AGCAATACGTAACTGAACGAAGTCCCTAACCCTAACCCTAA;
(SEQ ID NO: 15)
AGCAATACGTAACTGAACGAAGTTAACCCTAACCCTAACCC;
(SEQ ID NO: 16)
AGCAATACGTAACTGAACGAAGTCTAACCCTAACCCTAACC;
(SEQ ID NO: 17)
AGCAATACGTAACTGAACGAAGTCCTAACCCTAACCCTAAC;
(SEQ ID NO: 18)
AGCAATACGTAACTGAACGAAGTAACCCTAACCCTAACCCT;
and
(SEQ ID NO: 19)
AGCAATACGTAACTGAACGAAGTACCCTAACCCTAACCCTA.

These are shown in Table 14.

Any of the telomere adaptors used in the invention may comprise click chemistry groups to facilitate covalent attachment as discussed below.

### Splint Polynucleotide

Step (a) preferably further comprises hybridising a splint polynucleotide to the 5′ end of the telomere adaptor. This is possible because the 5′ end of the telomere adaptor is not hybridised to the overhanging strand at the end of the telomere. The splint polynucleotide may be any of the polynucleotides discussed above with reference to the telomere adaptor. The splint polynucleotide may be any of the lengths discussed above with reference to the 5′ of the telomere adaptor. The splint polynucleotide may be the same length as the 5′ end or may be a different length. All or part of the splint polynucleotide may hybridise to all or part of the 5′ end.

The splint polynucleotide typically specifically hybridises to the 5′ end of the telomere adaptor. Specific hybridisation is discussed above. The splint polynucleotide typically comprises or consists of a sequence at least 80% identical to the reverse complement of a part or portion of the sequence of 5′ end of the telomere adaptor. The splint polynucleotide preferably comprises or consists of a sequence at least about 85% identical, at least about 90%, at least about 95%, at least about 98% or at least about 99% identical to the reverse complement of a part or portion of the 5′ end of the telomere adaptor. The splint polynucleotide preferably comprises or consists of a sequence that is complementary to the reverse complement of a part or portion the 5′ end of the telomere adaptor. By complementary, it is meant the splint polynucleotide comprises or consists of a sequence which is 100% identical to the reverse complement of the part or portion 5′ end of the telomere adaptor. Complementarity is typically determined using canonical Watson-Crick base pairing. The part or portion of the 5′ end may be any length, such as at least about 5, at least about 10, at least about 15 or at least about 20 nucleotides in length.

The splint polynucleotide preferably comprises or consists of a sequence which specifically hybridises to a part or portion of, or all of, AGCAATACGTAACTGAACGAAGT (SEQ ID NO: 7). A preferred splint polynucleotide comprises or consists of ACTTCGTTCAGTTACGTATTGCT (SEQ ID NO: 8). This is in the 5′ to 3′ direction. SEQ ID NO: 8 is the reverse complement of SEQ ID NO: 7.

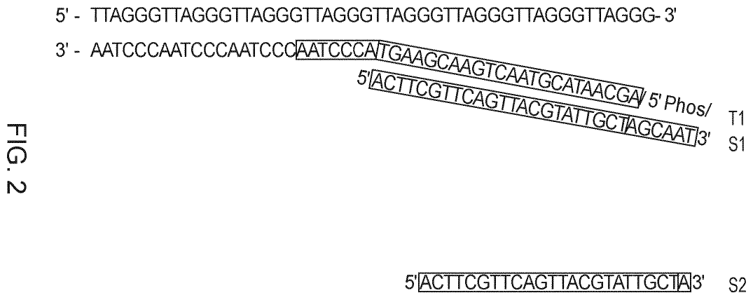

The splint polynucleotide is preferably compatible with a sequencing adaptor. The splint polynucleotide is typically adapted so it forms a 3′ overhang when hybridised to the 5′ end of the telomere adaptor. An example of this is shown in FIG. 2. The overhang may be any length such as at least about one nucleotide, at least about two nucleotides, at least about three nucleotides, at least about four nucleotides, at least about five nucleotides or at least about six nucleotides in length. The 3′ overhang preferably specifically hybridises to a part of the sequencing adaptor, such as an overhang on the sequencing adaptor. Specific hybridisation is discussed above. The 3′ overhang is preferably complementary to a part of the sequencing adaptor, such as an overhang on the sequencing adaptor. Suitable sequencing adaptors are discussed in more detail below.

The splint polynucleotide preferably comprises or consists of ACTTCGTTCAGTTACGTATTGCTAGCAAT (SEQ ID NO: 9) or ACTTCGTTCAGTTACGTATTGCTA (SEQ ID NO: 10). These are shown in Table 5 and specifically hybridise to a 5′ end having the sequence shown in SEQ ID NO: 7. The overhang on the sequencing adaptor preferably comprises ATTGCT.

The splint polynucleotide preferably comprises or consists of ACTTCGTTCGGTGACTTGAGGACAGCAAT (SEQ ID NO: 20). This is shown in Table 15. The overhang on the sequencing adaptor preferably comprises ATTGCT.

### Sequencing Adaptors

Step (a) preferably further comprises attaching a sequencing adaptor to the telomere adaptor and, if present, the splint polynucleotide, and step (b) preferably comprises using the sequencing adaptor to characterise the ligated, non-overhanging strand of the at least part of the telomere in the 5′ to 3′ direction. Step (a) preferably further comprises attaching a sequencing adaptor to the telomere adaptor and step (b) preferably comprises using the sequencing adaptor to characterise the ligated, non-overhanging strand of the at least part of the telomere in the 5′ to 3′ direction. Step (a) preferably further comprises attaching a sequencing adaptor to the telomere adaptor and the splint polynucleotide and step (b) preferably comprises using the sequencing adaptor to characterise the ligated, non-overhanging strand of the at least part of the telomere in the 5′ to 3′ direction. In all instances, the attachment is preferably covalent attachment. The sequencing adaptor may be ligated or annealed to the telomere adaptor and, if present, the splint polynucleotide. The sequencing adaptor is preferably attached to the telomere adaptor and, if present, splint polynucleotide by ligation. The sequencing adaptor may be attached to the telomere adaptor using click chemistry. Suitable click chemistry groups are discussed in more detail below.

Step (a) more preferably further comprises specifically hybridising a 3′ overhang formed by the splint polynucleotide hybridised to the telomere adaptor with an overhang on the sequencing adaptor and attaching, preferably covalently attaching, the sequencing adaptor to the telomere adaptor and splint polynucleotide. An example of this is shown in FIG. 3. The sequencing adaptor may be ligated or annealed to the telomere adaptor and splint polynucleotide. The sequencing adaptor is preferably attached to the telomere adaptor and splint polynucleotide by ligation.

A sequencing adaptor typically comprises a polynucleotide strand capable of being attached to the end of a target polynucleotide. The target polynucleotide is typically intended for characterisation in accordance with methods disclosed herein, and includes the telomere adaptor, the splint polynucleotide and/or the polynucleotide extension.

A sequencing adaptor may be added to both ends of the target polynucleotide. Alternatively, different adaptors may be added to the two ends of the target polynucleotide. An adaptor may be added to just one end of the target polynucleotide. Methods of adding adaptors to polynucleotides are known in the art. Adaptors may be attached to polynucleotides, for example, by ligation, by click chemistry, by tagmentation, by topoisomerisation or by any other suitable method.

An adaptor may be synthetic or artificial. Typically, an adaptor comprises a polymer as described herein. The adaptor preferably comprises a polynucleotide. An adaptor may comprise a single-stranded polynucleotide strand. An adaptor may comprise a double-stranded polynucleotide. A sequencing adaptor may comprise any of the polynucleotide discussed above with reference to the telomere adaptor and includes DNA, RNA, modified DNA (such as a basic DNA), RNA, PNA, LNA, BNA and/or PEG. Usually, the adaptor comprises single stranded and/or double stranded DNA or RNA.

A sequencing adaptor may be a Y adaptor. A Y adaptor is typically double stranded and comprises (a) at one end, a region where the two strands are hybridised together and (b), at the other end, a region where the two strands are not complementary. The non-complementary parts of the strands form overhangs. The hybridised stem of the adaptor typically attaches to the 5′ end of a first strand of a double-stranded polynucleotide and the 3′ end of a second strand of a double-stranded polynucleotide; or to the 3′ end of a first strand of a double-stranded polynucleotide and the 5′ end of a second strand of a double-stranded polynucleotide. The presence of a non-complementary region in the Y adaptor gives the adaptor its Y shape since the two strands typically do not hybridise to each other unlike the double stranded portion. The hybridised stem end of the Y adaptor may also comprise a short overhang that allows it to specifically hybridise to and be attached to the telomere adaptor, splint polynucleotide or polynucleotide extension.

Some of the methods of the invention use a polynucleotide binding protein to control the movement of a telomeric strand with respect to a nanopore. A polynucleotide binding protein may bind to an overhang of an adaptor such as a Y adaptor. A polynucleotide binding protein may bind to the double stranded region. A polynucleotide binding protein may bind to a single-stranded and/or a double-stranded region of the adaptor. A first polynucleotide binding protein may bind to the single-stranded region of such an adaptor and a second polynucleotide binding protein may bind to the double-stranded region of the adaptor.

The sequencing adaptor preferably comprises a membrane anchor or a pore anchor. The anchor may be attached to a polynucleotide that is complementary to and hence that is hybridised to the overhang to which a polynucleotide binding protein is bound.

One of the non-complementary strands of a sequencing adaptor, such as a Y adaptor, may comprise a leader sequence, which when contacted with a nanopore is capable of threading into the nanopore.

The leader sequence typically comprises a polymer such as a polynucleotide, for instance DNA or RNA, a modified polynucleotide (such as abasic DNA), PNA, LNA, polyethylene glycol (PEG) or a polypeptide. The leader sequence preferably comprises a single strand of DNA, such as a poly dT section. The leader sequence can be any length, but is typically 10 to 150 nucleotides in length, such as from 20 to 120, 30 to 100, 40 to 80 or 50 to 70 nucleotides in length.

The sequencing adaptor may be a hairpin loop adaptor. A hairpin loop adaptor is an adaptor comprising a single polynucleotide strand, wherein the ends of the polynucleotide strand are capable of hybridising to each other, or are hybridized to each other, and wherein the middle section of the polynucleotide forms a loop. Suitable hairpin loop adaptors can be designed using methods known in the art. Typically, the 3′ end of a hairpin loop adaptor attaches to the 5′ end of a first strand of a double-stranded polynucleotide and the 5′ end of the hairpin loop adaptor attaches to the 3′ end of a second strand of a double-stranded polynucleotide; or the 5′ end of a hairpin loop adaptor attaches to the 3′ end of a first strand of a double-stranded polynucleotide and the 3′ end of the hairpin loop adaptor attaches to the 5′ end of a second strand of a double-stranded polynucleotide. As explained in more detail below, a sequencing adaptor can be attached to a target polynucleotide in order to characterise the target polynucleotide.

Those skilled in the art will also appreciate that when the adaptor comprises a polynucleotide strand, the sequence of the adaptor is typically not determinative and can be controlled or chosen according to the polynucleotide binding protein and other experimental conditions such as any polynucleotide to be characterised. Exemplary sequences are provided solely by way of illustration in the examples. For example, the adaptor may comprise a sequence such as one or more of SEQ ID NOs: 21-26 or 28-33 in WO 2021/255476 (incorporated herein by reference in its entirety) or a polynucleotide sequence having at least 20%, such as at least 30%, e.g., at least 40% such as at least 50%, e.g., at least 60% such as at least 70%, e.g., at least 80%, for example at least 90% e.g., at least 95% sequence similarity or identity to one or more of SEQ ID NOs: 21-26 or 28-33 in WO 2021/255476 (incorporated herein by reference in its entirety). The sequence of the adaptor can typically be altered without negatively affecting the efficacy of the method of the invention.

A sequencing adaptor may comprise a loading site for loading the polynucleotide binding protein. The loading site may be for instance a single-stranded region which can targeted by the polynucleotide binding protein. The loading site may be a region of the sequencing adaptor to which an exogenous polynucleotide strand comprising the polynucleotide binding protein can bind in order to transfer the polynucleotide binding protein to the polynucleotide to be assessed in the method of the invention.

The polynucleotide binding protein if present may be provided on a sequencing adaptor. WO 2015/110813 and WO 2020/234612 describe the loading of polynucleotide binding proteins onto a target polynucleotide such as an adaptor and are hereby incorporated by reference in their entireties.

### Spacers

A polynucleotide, such as a telomere adaptor, a sequencing adaptor, a splint polynucleotide, or a polynucleotide extension, used in the invention may comprise one or more spacers, e.g., from about one to about 10 spacers, e.g., from about 1 to about 5 spacers, e.g., about 1, 2, 3, 4 or 5 spacers. The spacer may comprise any suitable number of spacer units. A spacer typically provides an energy barrier which impedes movement of a polynucleotide binding protein. For example, a spacer may impede movement of a polynucleotide binding protein by reducing the traction of the protein, e.g., using an abasic spacer. A spacer may physically block movement of the protein, for instance by introducing a bulky chemical group to physically impede the movement of the polynucleotide binding protein.

One or more spacers are typically included in the polynucleotide or in a sequencing adaptor to provide a distinctive signal when they pass through or across a nanopore. One or more spacers may be used to define or separate one or more regions of a polynucleotide, e.g., to separate an adaptor from the target polynucleotide.

A spacer may comprise a linear molecule, such as a polymer, e.g., a polypeptide or a polyethylene glycol (PEG). Typically, such a spacer has a different structure from the target polynucleotide. For instance, if the target polynucleotide is DNA, the or each spacer typically does not comprise DNA. In particular, if the target polynucleotide is deoxyribonucleic acid (DNA) or ribonucleic acid (RNA), the or each spacer preferably comprises peptide nucleic acid (PNA), glycerol nucleic acid (GNA), threose nucleic acid (TNA), locked nucleic acid (LNA) or a synthetic polymer with nucleotide side chains. A spacer may comprise one or more nitroindoles, one or more inosines, one or more acridines, one or more 2-aminopurines, one or more 2-6-diaminopurines, one or more 5-bromo-deoxyuridines, one or more inverted thymidines (inverted dTs), one or more inverted dideoxy-thymidines (ddTs), one or more dideoxy-cytidines (ddCs), one or more 5-methylcytidines, one or more 5-hydroxymethylcytidines, one or more 2′-O-Methyl RNA bases, one or more Iso-deoxycytidines (Iso-dCs), one or more Iso-deoxyguanosines (Iso-dGs), one or more C3 (OC₃H₆OPO₃) groups, one or more photo-cleavable (PC) [OC₃H₆—C(O)NHCH₂—C₆H₃NO₂—CH(CH₃)OPO₃] groups, one or more hexandiol groups, one or more spacer 9 (iSp9) [(OCH₂CH₂)₃OPO₃] groups, or one or more spacer 18 (iSp18) [(OCH₂CH₂)₆OPO₃] groups; or one or more thiol connections. A spacer may comprise any combination of these groups. Many of these groups are commercially available from IDT® (Integrated DNA Technologies®). For example, C3, iSp9 and iSp18 spacers are all available from IDT®. A spacer may comprise any number of the above groups as spacer units.

A spacer may comprise one or more chemical groups, e.g., one or more pendant chemical groups. The one or more chemical groups may be attached to one or more nucleobases in a sequencing adaptor. The one or more chemical groups may be attached to the backbone of a sequencing adaptor. Any number of appropriate chemical groups may be present, such as 2, 3, 4, 5, 6, 7, 8, 9, 10, 11, 12 or more. Suitable groups include, but are not limited to, fluorophores, streptavidin and/or biotin, cholesterol, methylene blue, dinitrophenols (DNPs), digoxigenin and/or anti-digoxigenin and dibenzylcyclooctyne groups.

A spacer may comprise one or more abasic nucleotides (i.e., nucleotides lacking a nucleobase), such as 2, 3, 4, 5, 6, 7, 8, 9, 10, 11, 12 or more abasic nucleotides. The nucleobase can be replaced by —H (idSp) or —OH in the abasic nucleotide. Abasic spacers can be inserted into target polynucleotides by removing the nucleobases from one or more adjacent nucleotides. For instance, polynucleotides may be modified to include 3-methyladenine, 7-methylguanine, 1,N6-ethenoadenine inosine or hypoxanthine and the nucleobases may be removed from these nucleotides using Human Alkyladenine DNA Glycosylase (hAAG). Alternatively, polynucleotides may be modified to include uracil and the nucleobases removed with Uracil-DNA Glycosylase (UDG). The one or more spacers preferably do not comprise any abasic nucleotides.

Suitable spacers can be designed or selected depending on the nature of the polynucleotide or sequencing adaptor, the polynucleotide binding protein, and the conditions under which the method is to be carried out.

### Tags

A polynucleotide, such as a telomere adaptor, a sequencing adaptor, a splint polynucleotide, or a polynucleotide extension, used in the invention may comprise a tag or tether. For example, a polynucleotide can bind to a tag on a nanopore, e.g., via its adaptor, and release at some point, e.g., during characterization of the polynucleotide by the nanopore. A strong non-covalent bond (e.g., biotin/avidin) is still reversible and can be useful in some embodiments of the methods described herein.

The pair of pore tag and sequencing adaptor can be configured such that the binding strength or affinity of a binding site on the polynucleotide (e.g., a binding site provided by an anchor or a leader sequence of an adaptor or by a capture sequence within the duplex stem of an adaptor) to a tag on a nanopore is sufficient to maintain the coupling between the nanopore and polynucleotide until an applied force is placed on it to release the bound polynucleotide from the nanopore.

The tags or tethers are preferably uncharged. This can ensure that the tags or tethers are not drawn into the nanopore under the influence of a potential difference.

One or more molecules that attract or bind the polynucleotide or adaptor may be linked to the detector (e.g., the pore). Any molecule that hybridizes to the adaptor and/or target polynucleotide may be used. The molecule attached to the pore may be selected from a PNA tag, a PEG linker, a short oligonucleotide, a positively charged amino acid and an aptamer. Pores having such molecules linked to them are known in the art. For example, pores having short oligonucleotides attached thereto are disclosed in Howarka et al (2001) Nature Biotech. 19: 636-639 and WO 2010/086620, and pores comprising PEG attached within the lumen of the pore are disclosed in Howarka et al (2000) J. Am. Chem. Soc. 122(11): 2411-2416.

A short oligonucleotide attached to the detector (e.g., a nanopore), which oligonucleotide comprises a sequence complementary to a sequence in the leader sequence or another single stranded sequence in the adaptor may be used to enhance capture of the target polynucleotide in the methods described herein.

The tag or tether may comprise or be an oligonucleotide (e.g., DNA, RNA, LNA, BNA, PNA, or morpholino). The oligonucleotide (e.g., DNA, RNA, LNA, BNA, PNA, or morpholino) can have about 10-30 nucleotides in length or about 10-20 nucleotides in length. The oligonucleotide (e.g., DNA, RNA, LNA, BNA, PNA, or morpholino) for use in the tag or tether can have at least one end (e.g., 3′- or 5′-end) modified for conjugation to other modifications or to a solid substrate surface including, e.g., a bead. The end modifiers may add a reactive functional group which can be used for conjugation. Examples of functional groups that can be added include, but are not limited to amino, carboxyl, thiol, maleimide, aminooxy, and any combinations thereof. The functional groups can be combined with different length of spacers (e.g., C3, C9, C12, Spacer 9 and 18) to add physical distance of the functional group from the end of the oligonucleotide sequence.

The tag or tether may comprise or be a morpholino oligonucleotide. The morpholino oligonucleotide can have about 10-30 nucleotides in length or about 10-20 nucleotides in length. The morpholino oligonucleotides can be modified or unmodified. For example, the morpholino oligonucleotide can be modified on the 3′ and/or 5′ ends of the oligonucleotides. Examples of modifications on the 3′ and/or 5′ end of the morpholino oligonucleotides include, but are not limited to 3′ affinity tag and functional groups for chemical linkage (including, e.g., 3′-biotin, 3′-primary amine, 3′-disulfide amide, 3′-pyridyl dithio, and any combinations thereof); 5′ end modifications (including, e.g., 5′-primary ammine, and/or 5′-dabcyl), modifications for click chemistry (including, e.g., 3′-azide, 3′-alkyne, 5′-azide, 5′-alkyne), and any combinations thereof.

The tag or tether may further comprise a polymeric linker, e.g., to facilitate coupling to a detector e.g., a nanopore. An exemplary polymeric linker includes, but is not limited to, polyethylene glycol (PEG). The polymeric linker may have a molecular weight of about 500 Da to about 10 kDa (inclusive), or about 1 kDa to about 5 kDa (inclusive). The polymeric linker (e.g., PEG) can be functionalized with different functional groups including, e.g., but not limited to maleimide, NHS ester, dibenzocyclooctyne (DBCO), azide, biotin, amine, alkyne, aldehyde, and any combinations thereof. The tag or tether may further comprise a 1 kDa PEG with a 5′-maleimide group and a 3′-DBCO group. The tag or tether may further comprise a 2 kDa PEG with a 5′-maleimide group and a 3′-DBCO group. The tag or tether may further comprise a 3 kDa PEG with a 5′-maleimide group and a 3′-DBCO group. The tag or tether may further comprise a 5 kDa PEG with a 5′-maleimide group and a 3′-DBCO group.

Other examples of a tag or tether include, but are not limited to His tags, biotin or streptavidin, antibodies that bind to analytes, aptamers that bind to analytes, analyte binding domains such as DNA binding domains (including, e.g., peptide zippers such as leucine zippers, single-stranded DNA binding proteins (SSB)), and any combinations thereof.

The tag or tether may be attached to the external surface of a nanopore, e.g., on the cis side of a membrane, using any methods known in the art. For example, one or more tags or tethers can be attached to the nanopore via one or more cysteines (cysteine linkage), one or more primary amines such as lysines, one or more non-natural amino acids, one or more histidines (His tags), one or more biotin or streptavidin, one or more antibody-based tags, one or more enzyme modification of an epitope (including, e.g., acetyl transferase), and any combinations thereof. Suitable methods for carrying out such modifications are well-known in the art. Suitable non-natural amino acids include, but are not limited to, 4-azido-L-phenylalanine (Faz) and any one of the amino acids numbered 1-71 in FIG. 1 of Liu C. C. and Schultz P. G., Annu. Rev. Biochem., 2010, 79, 413-444.

Where one or more tags or tethers are attached to a nanopore via cysteine linkage(s), the one or more cysteines can be introduced to one or more monomers that form the nanopore by substitution. The nanopore may be chemically modified by attachment of (i) Maleimides including diabromomaleimides such as: 4-phenylazomaleinanil, 1.N-(2-Hydroxyethyl)maleimide, N-Cyclohexylmaleimide, 1.3-Maleimidopropionic Acid, 1.1-4-Aminophenyl-1H-pyrrole,2,5,dione, 1.1-4-Hydroxyphenyl-1H-pyrrole,2,5,dione, N-Ethylmaleimide, N-Methoxycarbonylmaleimide, N-tert-Butylmaleimide, N-(2-Aminoethyl)maleimide, 3-Maleimido-PROXYL, N-(4-Chlorophenyl)maleimide, 1-[4-(dimethylamino)-3,5-dinitrophenyl]-1H-pyrrole-2,5-dione, N-[4-(2-Benzimidazolyl)phenyl]maleimide, N-[4-(2-benzoxazolyl)phenyl]maleimide, N-(1-naphthyl)-maleimide, N-(2,4-xylyl)maleimide, N-(2,4-difluorophenyl)maleimide, N-(3-chloro-para-tolyl)-maleimide, 1-(2-amino-ethyl)-pyrrole-2,5-dione hydrochloride, 1-cyclopentyl-3-methyl-2,5-dihydro-1H-pyrrole-2,5-dione, 1-(3-aminopropyl)-2,5-dihydro-1H-pyrrole-2,5-dione hydrochloride, 3-methyl-1-[2-oxo-2-(piperazin-1-yl)ethyl]-2,5-dihydro-1H-pyrrole-2,5-dione hydrochloride, 1-benzyl-2,5-dihydro-1H-pyrrole-2,5-dione, 3-methyl-1-(3,3,3-trifluropropyl)-2,5-dihydro-1H-pyrrole-2,5-dione, 1-[4-(methylamino)cyclohexyl]-2,5-dihydro-1H-pyrrole-2,5-dione trifluroacetic acid, SMILES O═C1C═CC(═O)N1CC=2C═CN═CC2, SMILES O═C1C═CC(═O)N1CN2CCNCC2, 1-benzyl-3-methyl-2,5-dihydro-1H-pyrrole-2,5-dione, 1-(2-fluorophenyl)-3-methyl-2,5-dihydro 1H-pyrrole-2,5-dione, N-(4-phenoxyphenyl)maleimide, N-(4-nitrophenyl)maleimide (ii) Iodocetamides such as: 3-(2-Iodoacetamido)-proxyl, N-(cyclopropylmethyl)-2-iodoacetamide, 2-iodo-N-(2-phenylethyl)acetamide, 2-iodo-N-(2,2,2-trifluoroethyl)acetamide, N-(4-acetylphenyl)-2-iodoacetamide, N-(4-(aminosulfonyl)phenyl)-2-iodoacetamide, N-(1,3-benzothiazol-2-yl)-2-iodoacetamide, N-(2,6-diethylphenyl)-2-iodoacetamide, N-(2-benzoyl-4-chlorophenyl)-2-iodoacetamide, (iii) Bromoacetamides: such as N-(4-(acetylamino)phenyl)-2-bromoacetamide, N-(2-acetylphenyl)-2-bromoacetamide, 2-bromo-n-(2-cyanophenyl)acetamide, 2-bromo-N-(3-(trifluoromethyl)phenyl)acetamide, N-(2-benzoylphenyl)-2-bromoacetamide, 2-bromo-N-(4-fluorophenyl)-3-methylbutanamide, N-Benzyl-2-bromo-N-phenylpropionamide, N-(2-bromo-butyryl)-4-chloro-benzenesulfonamide, 2-Bromo-N-methyl-N-phenylacetamide, 2-bromo-N-phenethyl-acetamide,2-adamantan-1-yl-2-bromo-N-cyclohexyl-acetamide, 2-bromo-N-(2-methylphenyl)butanamide, Monobromoacetanilide, (iv) Disulphides such as: aldrithiol-2, aldrithiol-4, isopropyl disulfide, 1-(Isobutyldisulfanyl)-2-methylpropane, Dibenzyl disulfide, 4-aminophenyl disulfide, 3-(2-Pyridyldithio)propionic acid, 3-(2-Pyridyldithio)propionic acid hydrazide, 3-(2-Pyridyldithio)propionic acid N-succinimidyl ester, am6amPDP1-βCD and (v) Thiols such as: 4-Phenylthiazole-2-thiol, Purpald, 5,6,7,8-tetrahydro-quinazoline-2-thiol.

The tag or tether may be attached directly to a nanopore or via one or more linkers. The tag or tether may be attached to the nanopore using the hybridization linkers described in WO 2010/086602 (incorporated herein by reference in its entirety). Alternatively, peptide linkers may be used. Peptide linkers are amino acid sequences. The length, flexibility and hydrophilicity of the peptide linker are typically designed such that it does not to disturb the functions of the monomer and pore. Preferred flexible peptide linkers are stretches of 2 to 20, such as 4, 6, 8, 10 or 16, serine and/or glycine amino acids. More preferred flexible linkers include (SG)₁, (SG)₂, (SG)₃, (SG)₄, (SG)₅ and (SG)₈ wherein S is serine and G is glycine. Preferred rigid linkers are stretches of 2 to 30, such as 4, 6, 8, 16 or 24, proline amino acids. More preferred rigid linkers include (P)₁₂ wherein P is proline.

Suitable pore tags are also described in WO 2018/100370, which describes non-hairpin methods for characterising double-stranded polynucleotides and is herein incorporated by reference in its entirety.

### Anchor

A polynucleotide, such as a telomere adaptor, a sequencing adaptor, a splint polynucleotide, or a polynucleotide extension, used in the invention may comprise a membrane anchor. The anchor typically assists in the characterisation of a target polynucleotide in accordance with the methods disclosed herein. For example, a membrane anchor may promote localisation of the selected polynucleotides around a nanopore.

The anchor may be a polypeptide anchor and/or a hydrophobic anchor that can be inserted into the membrane. The hydrophobic anchor is preferably a lipid, fatty acid, sterol, carbon nanotube, polypeptide, protein, or amino acid, for example cholesterol, palmitate, or tocopherol. The anchor may comprise thiol, biotin, or a surfactant.

The anchor may be biotin (for binding to streptavidin), amylose (for binding to maltose binding protein or a fusion protein), Ni-NTA (for binding to poly-histidine or poly-histidine tagged proteins) or peptides (such as an antigen).

The anchor preferably comprises a linker, or 2, 3, 4 or more linkers. Preferred linkers include, but are not limited to, polymers, such as polynucleotides, polyethylene glycols (PEGs), polysaccharides and polypeptides. These linkers may be linear, branched, or circular. For instance, the linker may be a circular polynucleotide. The adaptor may hybridise to a complementary sequence on a circular polynucleotide linker. The one or more anchors or one or more linkers may comprise a component that can be cut or broken down, such as a restriction site or a photolabile group. The linker may be functionalised with maleimide groups to attach to cysteine residues in proteins. Suitable linkers are described in WO 2010/086602 (incorporated herein by reference in its entirety).

The anchor is preferably cholesterol or a fatty acyl chain. For example, any fatty acyl chain having a length of from 6 to 30 carbon atom, such as hexadecanoic acid, may be used.

Examples of suitable anchors and methods of attaching anchors to adaptors are disclosed in WO 2012/164270 and WO 2015/150786 (incorporated herein by reference in their entireties).

The anchor may consist or comprise a hydrophobic modification to the polynucleotide or sequencing adaptor. The hydrophobic modification may comprise a modified phosphate group comprised within the polynucleotide or polynucleotide anchor. The hydrophobic modification may for example comprise a phosphorothioate such as a charge-neutralized alkyl-phosphorothioate (PPT) as described in Jones et al, J. Am. Chem. Soc. 2021, 143, 22, 8305, the entire contents of which are hereby incorporated by reference. Suitable alkyl groups include for example C₁-C₁₀ alkyl groups such as C₂-C₆ alkyl groups, e.g., methyl, ethyl, propyl, butyl, pentyl and hexyl groups. Incorporation of the charge-neutralized alkyl-phosphorothioate into a polynucleotide allows for the polynucleotide to anchor to a hydrophobic region such as a lipid bilayer.

### Polynucleotide Extension

In an alternative to using the splint polynucleotides, step (a) preferably further comprises ligating or covalently attaching a polynucleotide extension to the telomere adaptor. The polynucleotide extension is typically ligated or covalently attached to the 5′ end of the telomere adaptor. The polynucleotide extension may be any of the polynucleotides discussed above with reference to the telomere adaptor. The polynucleotide extension may be any length, including any of the lengths discussed above with reference to the 5′ of the telomere adaptor. The polynucleotide extension is preferably covalently attached to the telomere adaptor using click chemistry. Suitable click chemistries are known in the art and discussed herein.

A preferred polynucleotide extension comprises or consists of GCTTGGGTGTTTAACC (SEQ ID NO: 11). This is used in the click adaptors of Table 7. The polynucleotide extension may comprise or further comprise one or more, such as two or more, three or more or four or more, universal nucleotides, such as Int 5-Nitroindole (i5NiTInd). The one or more universal nucleotides are typically at the 3′ end of the polynucleotide extension. The polynucleotide extension may be ligated or covalently attached to the telomere adaptor using the one or more universal nucleotides.

The method of the invention preferably comprises in step (a) ligating a polynucleotide telomere adaptor to the 5′ end of the non-overhanging strand at the end of the telomere wherein the 3′ end of the adaptor specifically hybridises to the first part of the overhanging strand and the 5′ end of the adaptor does not hybridise to the opposite part of the overhanging strand and wherein the telomere adaptor comprises a polynucleotide extension at the 5′ end. Any of the embodiments discussed above with reference to telomere adaptors and polynucleotide extensions equally apply to this “extended” telomere adaptor embodiment.

The extended telomere adaptor preferably comprises or consists of a sequence shown in SEQ ID NO: 11 covalently attached to any one of SEQ ID NOs: 1-6. The extended telomere adaptor preferably comprises or consists of a sequence shown in SEQ ID NO: 11 covalently attached to any one of SEQ ID NOs: 14-19. The two sequences are preferably covalently attached by one or more, such as two or more, three or more or four or more, universal nucleotides, such as Int 5-Nitroindole (i5NiTInd). The two sequences are more preferably covalently attached by 4 i5NiTInds. The extended telomere adaptor preferably comprises or consists of a sequence shown in any one of SEQ ID NOs: 25-30. The extended telomere adaptor preferably comprises a click chemistry group, such as DBCOTEG, at the 5′ end. Examples of these extended telomere adaptors are shown in Table 7.

The method preferably comprises before step (a) contacting the telomere with a population of six extended telomere adaptors which comprise or consist of the following sequences:

SEQ ID NO: 11 covalently attached to
(SEQ ID NO: 1)
AGCAATACGTAACTGAACGAAGTACCCTAA;
SEQ ID NO: 11 covalently attached to
(SEQ ID NO: 2)
AGCAATACGTAACTGAACGAAGTAACCCTA;
SEQ ID NO: 11 covalently attached to
(SEQ ID NO: 3)
AGCAATACGTAACTGAACGAAGTTAACCCT;
SEQ ID NO: 11 covalently attached to
(SEQ ID NO: 4)
AGCAATACGTAACTGAACGAAGTCTAACCC;
SEQ ID NO: 11 covalently attached to
(SEQ ID NO: 5)
AGCAATACGTAACTGAACGAAGTCCTAACC;
and
SEQ ID NO: 11 covalently attached to
(SEQ ID NO: 6)
AGCAATACGTAACTGAACGAAGTCCCTAAC.

The method preferably comprises before step (a) contacting the telomere with a population of six extended telomere adaptors which comprise or consist of the following sequences:

(SEQ ID NO: 25)
GCTTGGGTGTTTAACCNNNNAGCAATACGTAACTGAACGAAGTACCCTA
A;
(SEQ ID NO: 26)
GCTTGGGTGTTTAACCNNNNAGCAATACGTAACTGAACGAAGTAACCCT
A;
(SEQ ID NO: 27)
GCTTGGGTGTTTAACCNNNNAGCAATACGTAACTGAACGAAGTTAACCC
T;
(SEQ ID NO: 28)
GCTTGGGTGTTTAACCNNNNAGCAATACGTAACTGAACGAAGTCTAACC
C;
(SEQ ID NO: 29)
GCTTGGGTGTTTAACCNNNNAGCAATACGTAACTGAACGAAGTCCTAAC
C;
and
(SEQ ID NO: 30)
GCTTGGGTGTTTAACCNNNNAGCAATACGTAACTGAACGAAGTCCCTAA
C,
wherein N is Int 5-Nitroindole (i5NiTInd).

The method preferably comprises before step (a) contacting the telomere with a population of six extended telomere adaptors which comprise or consist of the following sequences:

SEQ ID NO: 11 covalently attached to
(SEQ ID NO: 14)
AGCAATACGTAACTGAACGAAGTCCCTAACCCTAACCCTAA;
SEQ ID NO: 11 covalently attached to
(SEQ ID NO: 15)
AGCAATACGTAACTGAACGAAGTTAACCCTAACCCTAACCC;
SEQ ID NO: 11 covalently attached to
(SEQ ID NO: 16)
AGCAATACGTAACTGAACGAAGTCTAACCCTAACCCTAACC;
SEQ ID NO: 11 covalently attached to
(SEQ ID NO: 17)
AGCAATACGTAACTGAACGAAGTCCTAACCCTAACCCTAAC;
SEQ ID NO: 11 covalently attached to
(SEQ ID NO: 18)
AGCAATACGTAACTGAACGAAGTAACCCTAACCCTAACCCT;
and
SEQ ID NO: 11 covalently attached to
(SEQ ID NO: 19)
AGCAATACGTAACTGAACGAAGTACCCTAACCCTAACCCTA.

The two sequences in the extended adaptors are preferably covalently attached by one or more, such as two or more, three or more or four or more, universal nucleotides, such as Int 5-Nitroindole (i5NiTInd). The two sequences in the extended adaptors are more preferably covalently attached by 4 i5NiTInds. The extended telomere adaptors preferably comprise a click chemistry group, such as DBCOTEG, at their 5′ ends. Examples of these extended adaptors are shown in Table 7 and FIG. 4.

The polynucleotide extension preferably comprises a sequencing adaptor and step (b) preferably comprises using the sequencing adaptor to characterise the ligated, non-overhanging strand of the at least part of the telomere in the 5′ to 3′ direction from the end of the telomere. Step (a) preferably further comprises attaching, such as covalently attaching, a sequencing adaptor to the polynucleotide extension and step (b) preferably comprises using the sequencing adaptor to characterise the ligated, non-overhanging strand of the at least part of the telomere in the 5′ to 3′ direction. Step (a) more preferably further comprises specifically hybridising a 5′ overhang on the sequencing adaptor to the polynucleotide extension and attaching, such as covalently attaching, the sequencing adaptor to the polynucleotide extension. In any of these sequencing adaptor embodiments, a polynucleotide extension may be ligated or covalently attached to the telomere adaptor or the method may use extended telomere adaptors as defined above. The sequencing adaptor may be ligated or annealed to the polynucleotide extension. The sequencing adaptor is preferably attached to the polynucleotide extension by ligation. The polynucleotide extension is preferably covalently attached to the sequencing adaptor using click chemistry. The polynucleotide extension preferably comprises a click chemistry group, such as DBCOTEG, group at its 5′ end and is attached to the sequencing adaptor using click chemistry. Other possible click chemistry groups are discussed in more detail below.

### Biotin Enrichment

The telomere adaptor(s), including the extended telomere adaptors, preferably comprise(s) biotin. The biotin is preferably at the 5′ end of the telomere adaptor(s) or extended telomere adaptor(s). An example of this is shown in FIG. 6. In such embodiments, step (a) preferably further comprises using the biotin to enrich the ligated, non-overhanging strand of the at least part of the telomere. Suitable methods for biotin-based enrichment are known in the art. For instance, the telomere adaptor(s) preferably comprise(s) biotin and a surface, such as a bead, comprises avidin/or streptavidin and the surface may be used to enrich the ligated, non-overhanging strand of the at least part of the telomere.

### Linkers or Spacers

One or more linkers or spacers are preferably present between the 3′ end of the telomere adaptor and the 5′ end of the telomere adaptor. The one or more linkers or spacers are preferably flexible. The telomere adaptor may comprise any number of one or more spacers, e.g., from about one to about 10 spacers, e.g., from about 1 to about 5 spacers, e.g., about 1, 2, 3, 4 or 5 spacers. The spacer may comprise any suitable number of spacer units. A spacer typically provides an energy barrier which impedes movement of a polynucleotide binding protein. For example, a spacer may impede movement of a polynucleotide binding protein by reducing the traction of the protein, e.g., using an abasic spacer. A spacer may physically block movement of the protein, for instance by introducing a bulky chemical group to physically impede the movement of the polynucleotide binding protein. Suitable spacers and their uses are discussed above with reference to sequencing adaptors and these equally apply to the telomere adaptor.

### Two-Way Characterisation

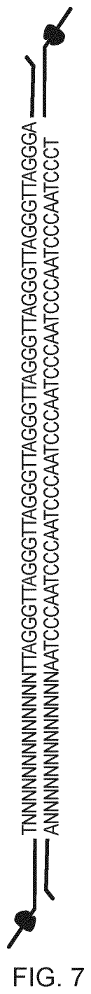

The method of the invention preferably further comprises characterising the non-ligated, overhanging strand of the at least part of the telomere in the 5′ to 3′ direction to the end of the telomere. An example of this is shown in FIG. 7. Any method may be used to characterise the non-ligated, overhanging strand, including any of the method discussed below. The same methods or different methods may be used to characterise the ligated, non-overhanging strand and the non-ligated, overhanging stand. The same method is preferably used to characterise the ligated, non-overhanging strand and the non-ligated, overhanging stand.

The method of the invention preferably further comprises using a polymer-guided effector protein, such as a Cas9 protein, to create a double stranded break at the opposite end of the at least part of the telomere from the telomere end and attaching a sequencing adaptor to the opposite end and using the sequencing adaptor to characterise the non-ligated, overhanging strand of the at least part of the telomere in the 5′ to 3′ direction to the end of the telomere. The sequencing adaptor may be ligated or annealed to the opposite end. The sequencing adaptor may be any of those discussed above. Attaching a Y sequencing adaptor to a double stranded break is known in the art.

The invention also provides a method for characterising at least part of a telomere, the method comprising (a) ligating a polynucleotide telomere adaptor to the 5′ end of the non-overhanging strand at the end of the telomere wherein the 3′ end of the adaptor is complementary to the first part of the overhanging strand and the 5′ end of the adaptor is not complementary to the opposite part of the overhanging strand, (b) using a polymer-guided effector protein, such as a Cas9 protein, to create a double stranded break at the opposite end of the at least part of the telomere from the telomere end and attaching a sequencing adaptor to the opposite end and (c) using the telomere adaptor to characterise the ligated non-overhanging strand of at least part of the telomere in the 5′ to 3′ direction from the end of the telomere and using the sequencing adaptor to characterise the non-ligated overhanging strand of the at least part of the telomere in the 5′ to 3′ direction to the end of the telomere.

### Polymer-Guided Effector Protein

The polymer-guided effector protein may be any protein that binds to the opposite end of the at least part of the telomere via a guide polymer. The polymer-guided effector protein may, by way of non-limiting examples, bind to or be attached to a guide oligonucleotide, such as an aptamer, or a guide polypeptide, such as an antibody, which binds to part of the at least part of the telomere.

The polymer-guided effector protein is preferably a polynucleotide-guided effector protein. In this embodiment, the guide polymer is preferably a guide polynucleotide. The polynucleotide-guided effector protein may be any protein that binds to or is attached to a guide polynucleotide and which binds to the opposite end of the at least part of the telomere, preferably a target polynucleotide sequence at the opposite end at least part of the telomere, to which the guide polynucleotide binds. The polynucleotide-guided effector protein preferably comprises a target polynucleotide sequence recognition domain and at least one nuclease domain. The recognition domain binds a guide polynucleotide (e.g., RNA) and a target polynucleotide (e.g., DNA). The polynucleotide-guided effector protein may contain one nuclease domain that cuts one or both strands of a double stranded polynucleotide or may contain two nuclease domains wherein a first nuclease domain is positioned for cleavage of one strand of the target polynucleotide sequence and a second nuclease domains is positioned for cleavage of the complementary strand of the target polynucleotide sequence. The nuclease domains may be active or inactive. For example, the nuclease domain or one or both of the two nuclease domains may be inactivated by mutation.

The guide polynucleotide may be a guide RNA, a guide DNA, or a guide containing both DNA and RNA. The guide polynucleotide is preferably a guide RNA. Therefore, the polynucleotide-guided effector protein is preferably an RNA-guided effector protein.

The RNA-guided effector protein may be any protein that binds to or is attached to the guide RNA. The RNA-guided effector protein typically binds to a region of guide RNA that is not the region of guide RNA which binds to the target polynucleotide sequence. For example, where the guide RNA comprises crRNA and tracrRNA, the RNA-guided effector protein typically binds to the tracrRNA and the crRNA typically binds to the opposite end of the at least part of the telomere, also known as a target polynucleotide sequence. The RNA-guided effector protein preferably also binds to a target polynucleotide sequence. The region of the guide RNA that binds to the target polynucleotide sequence may also bind to the RNA-guided effector protein. The RNA-guided effector protein typically binds to a double stranded region of the target polynucleotide sequence. The region of the target polynucleotide sequence to which the RNA-guided effector protein binds is typically located close to the sequence to which the guide RNA hybridizes. The guide RNA and RNA-guided effector protein typically form a complex, which complex then binds to the target polynucleotide sequence at a site determined by the sequence of the guide RNA.

The RNA-guided effector protein may bind upstream or downstream of the sequence to which the guide RNA binds. For example, the RNA-guided effector protein may bind to a protospacer adjacent motif (PAM) in DNA located next to the sequence to which the guide RNA binds. A PAM is a short (less than 10, typically a 2 to 6 base pair) sequence, such as 5′-NGG-3′ (wherein N is any base), 5′-NGA-3′, 5′-YG-3′ (wherein Y is a pyrimidine), 5′TTN-3′ or 5′-YTN-3′. Different RNA-guided effector proteins bind to different PAMs. RNA-guided effector proteins may bind to a target polynucleotide sequence which does not comprise a PAM, in particular, where the target is RNA or a DNA/RNA hybrid.

The RNA-guided effector protein is typically a nuclease, such as an RNA-guided endonuclease. The RNA-guided effector protein is typically a Cas protein. The RNA-guided effector protein may be Cas, Csn2, Cpf1, Csf1, Cmr5, Csm2, Csy1, Cse1 or C2c2. The Cas protein may be Cas3, Cas 4, Cas8a, Cas8b, Cas8c, Cas9, Cas10 or Cas10d. Preferably, the Cas protein is Cas9. Cas, Csn2, Cpf1, Csf1, Cmr5, Csm2, Csy1 or Cse1 is preferably used where the target polynucleotide sequence comprises a double stranded DNA region. C2c2 is preferably used where the target polynucleotide sequence comprises a double stranded RNA region.

A DNA-guided effector protein, such as proteins from the RecA family may be used to target DNA. Examples of proteins from the RecA family that may be used are RecA, RadA and Rad51. The nuclease activity of the RNA-guided endonuclease may be disabled. One or more of the catalytic nuclease sites of the RNA-guided endonuclease may be inactivated. For example, where the RNA-guided endonuclease comprises two catalytic nuclease sites, one or both of the catalytic sites may be inactivated. Typically, one of the catalytic sites will cut one strand of the polynucleotide to which it specifically binds and the other catalytic site will cut the opposite strand of the polynucleotide. Therefore, the RNA-guided endonuclease may cut both strands, one strand or neither strand of a double stranded region of a target polynucleotide. The RNA-guided endonuclease preferably cuts both strands of the at least part of the telomere.

The polynucleotide-guided effector protein is preferably Cas9. Cas9 has a bi-lobed, multi-domain protein structure comprising target recognition and nuclease lobes. The recognition lobe binds guide RNA and DNA. The nuclease lobe contains the HNH and RuvC nuclease domains which are positioned for cleavage of the complementary and non-complementary strands of the target DNA. The structure of Cas 9 is detailed in Nishimasu, H., et al., (2014) Crystal Structure of Cas9 in Complex with Guide RNA and Target DNA. Cell 156, 935-949. The relevant PDB reference for Cas9 is 5F9R (Crystal structure of catalytically active Streptococcus pyogenes CRISPR-Cas9 in complex with single-guided RNA and double-stranded DNA primed for target DNA cleavage).

The Cas9 may be an ‘enhanced specificity’ Cas9 that shows reduced off-target binding compared to wild-type Cas9. An example of such an ‘enhanced specificity’ Cas9 is S. pyogenes Cas9 D10A/H840A/K848A/K1003A/R1060A. ONLP12296 is the amino acid sequence of S. pyogenes Cas9 D10A/H840A/K848A/K1003A/R1060A having a C-terminal Twin-Strep-tag with TEV-cleavable linker.

Catalytic sites of an RNA-guided endonuclease may be inactivated by mutation. The mutation may be a substitution, insertion, or deletion mutation. For example, one or more, such as 2, 3, 4, 5, or 6 amino acids may be substituted or inserted into or deleted from the catalytic site. The mutation is preferably a substitution insertion, more preferably substitution if a single amino acid at the catalytic site. The skilled person will be readily able to identify the catalytic sites of an RNA-guided endonuclease and mutations that inactivate them. For example, where the RNA-guided endonuclease is Cas9, one catalytic site may be inactivated by a mutation at D10 and the other by a mutation at H640.

An inactivated (‘dead’) polynucleotide-guided effector protein that does not cut the target polynucleotide sequence and so shows no directionality bias. An active (‘live’) polynucleotide-guided effector protein that cuts the target polynucleotide sequence may remain bound to just one of the two ends of the cut site and so may show some directionality bias.

The polymer-guided effector protein may be specifically modified for use in a method of the invention. The protein may comprise an anchor capable of coupling to a membrane, such as cholesterol. The polymer-guided effector protein preferably has a binding moiety capable of coupling to a surface attached thereto. The surface is preferably the surface of beads.

### Guide Polymer

The guide polymer is capable of binding to the opposite end of the at least part of the telomere and mediating the binding of the polymer-guided effector protein to the opposite end of the at least part of the telomere. The guide polymer may have any structure that enables it to bind to the opposite end of the at least part of the telomere, also known as the target polynucleotide sequence. It may be any of the polymers discussed above with reference to the telomere adaptor. The guide polymer is preferably an oligonucleotide, a polynucleotide, a polypeptide, a protein, an oligosaccharide, or a polysaccharide. The guide polypeptide or protein is preferably a zinc finger binding protein, a transcription activator-like effector (TALE), transcription factor, restriction enzyme, DNA-binding protein or enzyme, an antibody, or an antibody fragment. Suitable antibody fragments are known in the art and include, but are not limited to, Fab, Fab′, (Fab′)2, FV, scFv, diabody, triabody, tetrabody, Bis-scFv, minibody, Fab2 and Fab3. The guide oligonucleotide is preferably an aptamer.

The guide polymer may bind to the polymer-guided effector protein. In this context, the guide polymer may bind to polymer-guided effector protein, may be bound by the polymer-guided effector protein or both. The guide polymer may be attached to the polymer-guided effector protein. Suitable methods are known in the art for attaching the guide polymer to the polymer-guided effector protein, for instance using streptavidin/biotin. The polymer-guided effector is preferably covalently attached to the guide polymer. Covalent attachment, for instance using click chemistry, is discussed in WO 2018/060740 (incorporated herein by reference in its entirety).

The guide polymer is preferably a guide polynucleotide. In this instance, the polymer-guided effector protein is preferably a polynucleotide-guided effector protein. The guide polynucleotide preferably comprises a sequence that is capable of binding to the opposite end of the at least part of the telomere or specifically hybridising to the target polynucleotide sequence. The guide polynucleotide preferably comprises a nucleotide sequence that binds to a sequence at the opposite end of the at least part of the telomere and a nucleotide sequence that binds to the polynucleotide-guided effector protein. The guide polynucleotide preferably comprises a nucleotide sequence that specifically hybridises to a sequence at the opposite end of the at least part of the telomere and a nucleotide sequence that binds to the polynucleotide-guided effector protein. The guide polynucleotide may have any structure that enables it to specifically bind/hybridise to the opposite end of the at least part of the telomere and bind to a polynucleotide-guided effector protein.

The guide polynucleotide typically specifically hybridizes to a sequence of about 20 nucleotides in the target polynucleotide sequence. The sequence to which the guide polynucleotide binds may be from about 10 to about 40, such as from about 15 to about 30, preferably from about 18 to about 25, such as about 19, 20, 21, 22, 23 or 24 nucleotides. The guide polynucleotide is typically complementary to one strand of the opposite end of the at least part of the telomere. The guide polynucleotide preferably comprises a nucleotide sequence of from about 10 to about 40, such as from about 15 to about 30, preferably from about 18 to about 25, such as about 19, 20, 21, 22, 23 or 24, nucleotides that is complementary to the sequence of, or to a sequence in, the target polynucleotide sequence. The degree of complementarity is preferably exact.

The guide polynucleotide is preferably guide RNA. The guide RNA may be complementary to a region in the target polynucleotide sequence that is 5′ to a PAM. This is preferred where the target polynucleotide comprises DNA, particularly where the RNA effector protein is Cas9 or Cpf1. The guide RNA may be complementary to a region in the target polynucleotide sequence that is flanked by a guanine. This is preferred where the target polynucleotide comprises RNA, particularly where the RNA effector protein is C2c2.

The guide RNA may have any structure that enables it to bind to the target polynucleotide sequence and to an RNA-guided effector protein. The guide RNA may comprise a crRNA that binds to a sequence in the target polynucleotide sequence and a tracrRNA. The tracrRNA typically binds to the RNA-guided effector protein. Typical structures of guide RNAs are known in the art. For example, the crRNA is typically a single stranded RNA and the tracrRNA typically has a double stranded region of which one strand is attached to the 3′ end of the crRNA and a part that forms a hairpin loop at the 3′ end of the strand that is not attached to the crRNA. The crRNA and tracrRNA may be transcribed in vitro as a single piece sgRNA. The guide RNA is preferably a sgRNA

The guide RNA may comprise other components, such as additional RNA bases or DNA bases or other nucleobases. The RNA and DNA bases in the guide RNA may be natural bases or modified bases. A guide DNA may be used in place of a guide RNA, and a DNA-guided effector protein used instead of an RNA-guided effector protein. The used of a guide DNA and a DNA-guided effector protein may be preferred where the target polynucleotide is RNA.

### Characterisation

Step (b) comprises using the telomere adaptor to characterise the ligated, non-overhanging strand of the at least part of the telomere in the 5′ to 3′ direction from the end of the telomere. The method preferably also comprises using a sequencing adaptor to characterise the non-ligated, overhanging strand of the at least part of the telomere in the 5′ to 3′ direction to the end of the telomere. Any method of characterisation may be used. The method preferably uses next generation sequencing (NGS).

The ligated, non-overhanging strand and/or the non-ligated, overhanging strand is/are preferably moved with respect to a detector such as a nanopore. The detector may be selected from (i) a zero-mode waveguide, (ii) a field-effect transistor, optionally a nanowire field-effect transistor; (iii) an AFM tip; (iv) a nanotube, optionally a carbon nanotube; and (v) a nanopore. Preferably, the detector is a nanopore.

The ligated, non-overhanging strand and/or the non-ligated, overhanging strand may be characterised in the method of the invention in any suitable manner. The ligated, non-overhanging strand and/or the non-ligated, overhanging strand is/are preferably characterised by detecting an ionic current or optical signal as it/they move(s) with respect to a nanopore. This is described in more detail herein. The method is amenable to these and other methods of characterising polynucleotides.

In another non-limiting example, the ligated, non-overhanging strand and/or the non-ligated, overhanging strand is/are characterised by detecting the by-products of a polynucleotide-processing reaction, such as a sequencing by synthesis reaction. The method may thus involve detecting the product of the sequential addition of (poly)nucleotides by an enzyme such as a polymerase to the nucleic acid strand. The product may be a change in one or more properties of the enzyme such as in the conformation of the enzyme. Such methods may thus comprise subjecting an enzyme such as polymerase or a reverse transcriptase to the polynucleotide(s) under conditions such that the template-dependent incorporation of nucleotide bases into a growing oligonucleotide strand causes conformational changes in the enzyme in response to sequentially encountering template strand nucleic acid bases and/or incorporating template-specified natural or analog bases (i.e., an incorporation event), detecting the conformational changes in the enzyme in response to such incorporation events, and thereby detecting the sequence of the template strand. In such methods the polynucleotide strand may be moved in accordance with the method of the invention. Such methods may involve detecting and/or measuring incorporation events using methods known to those skilled in the art, such as those described in US 2017/0044605.

In another embodiment, by-products may be labelled so that a phosphate labelled species is released upon the addition of a nucleotide to a synthesised nucleic acid strand that is complementary to the template strand, and the phosphate labelled species is detected e.g., using a detector as described herein. The polynucleotide being characterised in this way may be moved in accordance with the methods herein. Suitable labels may be optical labels that are detected using a nanopore, or a zero-mode wave guide, or by Raman spectroscopy, or other detectors. Suitable labels may be non-optical labels that are detected using a nanopore, or other detectors.

In another approach, nucleoside phosphates (nucleotides) are not labelled and upon the addition of a nucleotide to a synthesised nucleic acid strand that is complementary to the template strand, a natural by-product species is detected. Suitable detectors may be ion-sensitive field-effect transistors, or other detectors.

These and other detection methods are suitable for use in the methods described herein. Any suitable measurements can be taken using a detector as the polynucleotide moves with respect to the detector.

### Nanopore Characterisation

The ligated, non-overhanging strand and/or the non-ligated, overhanging strand is/are preferably characterised using a nanopore.

The method preferably comprises (i) contacting the ligated, non-overhanging strand and/or the non-ligated, overhanging strand with a nanopore such that the ligated, non-overhanging strand and/or the non-ligated, overhanging strand move(s) with respect to the nanopore and (ii) taking one or more measurements as the ligated, non-overhanging strand and/or the non-ligated, overhanging strand move(s) with respect to the nanopore wherein the measurements are indicative of one or more characteristics of the ligated, non-overhanging strand and/or the non-ligated, overhanging strand and thereby characterising the ligated, non-overhanging strand and/or the non-ligated, overhanging strand. The one or more characteristics are preferably selected from (i) the length of the ligated, non-overhanging strand and/or the non-ligated, overhanging strand, (ii) the identity of the ligated, non-overhanging strand and/or the non-ligated, overhanging strand, (iii) the sequence of the ligated, non-overhanging strand and/or the non-ligated, overhanging strand, (iv) the secondary structure of the ligated, non-overhanging strand and/or the non-ligated, overhanging strand and (v) whether or not the ligated, non-overhanging strand and/or the non-ligated, overhanging strand is modified. The ligated, non-overhanging strand and/or the non-ligated, overhanging strand may be modified by methylation, by oxidation, by damage, with one or more proteins or with one or more labels, tags, or spacers. The one or more characteristics of the ligated, non-overhanging strand and/or the non-ligated, overhanging strand are preferably measured by electrical measurement and/or optical measurement. The electrical measurement is preferably a current measurement, an impedance measurement, a tunnelling measurement, or a field effect transistor (FET) measurement.

The method more preferably comprises (i) contacting the ligated, non-overhanging strand and/or the non-ligated, overhanging strand with a nanopore such that the ligated, non-overhanging strand and/or the non-ligated, overhanging strand moves through the nanopore and (ii) measuring the current moving through the nanopore as the ligated, non-overhanging strand and/or the non-ligated, overhanging strand moves through the nanopore wherein the current is indicative of one or more characteristics of the ligated, non-overhanging strand and/or the non-ligated, overhanging strand and thereby characterising the ligated, non-overhanging strand and/or the non-ligated, overhanging strand. The one or more characteristics may be any of those described above.

The movement of the ligated, non-overhanging strand and/or the non-ligated, overhanging strand with respect to the nanopore or through the nanopore is preferably controlled using a polynucleotide binding protein. The use of such proteins in nanopore sequencing is known. Examples of suitable proteins are discussed in more detail below.

The invention also provides a method for characterising at least part of a telomere, the method comprising (a) ligating a polynucleotide telomere adaptor to the 5′ end of the non-overhanging strand at the end of the telomere wherein the 3′ end of the specifically hybridises to the first part of the overhanging strand and the 5′ end of the adaptor does not hybridise to the opposite part of the overhanging strand, (b) using a polymer-guided effector protein, such as a Cas9 protein, to create a double stranded break at the opposite end of the at least part of the telomere from the telomere end and attaching a sequencing adaptor to the opposite end, (c) contacting the telomere adaptor with a nanopore such that the ligated, non-overhanging strand moves with respect to the nanopore, taking one or more measurements as the ligated, non-overhanging strand moves with respect to the nanopore wherein the measurements are indicative of one or more characteristics of the ligated, non-overhanging strand and thereby characterising the ligated, non-overhanging strand, and (d) contacting the sequencing adaptor with a nanopore such that the non-ligated, overhanging strand moves with respect to the nanopore, taking one or more measurements as the non-ligated, overhanging strand moves with respect to the nanopore, wherein the measurements are indicative of one or more characteristics of the non-ligated, overhanging strand and thereby characterising the non-ligated, overhanging strand. The ligated, non-overhanging strand and the non-ligated, overhanging strand preferably move through the nanopore. The method preferably comprises measuring the current moving through the nanopore wherein the current is indicative of one or more characteristics of the ligated, non-overhanging strand and the non-ligated, overhanging strand. The movement of the ligated, non-overhanging strand and the non-ligated, overhanging strand with respect to the nanopore/through the nanopore is preferably controlled using a polynucleotide binding protein. The telomere adaptor and the sequencing adaptor may be any of those discussed above.

Any suitable nanopore can be used. The nanopore is preferably a transmembrane pore. A transmembrane pore is a structure that crosses the membrane to some degree. It permits hydrated ions driven by an applied potential to flow across or within the membrane. The transmembrane pore typically crosses the entire membrane so that hydrated ions may flow from one side of the membrane to the other side of the membrane. However, the transmembrane pore does not have to cross the membrane. It may be closed at one end. For instance, the pore may be a well, gap, channel, trench or slit in the membrane along which or into which hydrated ions may flow.

The nanopore typically has a first opening and a second opening. The first opening is typically the cis opening and the second opening is typically the trans opening. However, the first opening may be the trans opening and the second opening may be the cis opening. Any polynucleotide binding protein used in the method of the invention is typically provided at the first opening of the nanopore and thus controls the movement of the target polynucleotide in the direction from the second opening of the nanopore towards the first opening of the nanopore.

Any transmembrane pore may be used in the method of the invention. The pore may be biological or artificial. Suitable pores include, but are not limited to, protein pores, polynucleotide pores and solid-state pores. The pore may be a DNA origami pore (Langecker et al., Science, 2012; 338: 932-936). Suitable DNA origami pores are disclosed in WO2013/083983.

The nanopore is preferably a transmembrane protein pore. A transmembrane protein pore is a polypeptide or a collection of polypeptides that permits hydrated ions, such as polynucleotide, to flow from one side of a membrane to the other side of the membrane. In the method of the invention, the transmembrane protein pore is capable of forming a pore that permits hydrated ions driven by an applied potential to flow from one side of the membrane to the other. The transmembrane protein pore preferably permits polynucleotides to flow from one side of the membrane, such as a triblock copolymer membrane, to the other. The transmembrane protein pore allows a polynucleotide to be moved through the pore.

The nanopore may be a transmembrane protein pore which is a monomer or an oligomer. The pore is preferably made up of several repeating subunits, such as at least about 6, at least about 7, at least about 8, at least about 9, at least about 10, at least about 11, at least about 12, at least about 13, at least about 14, at least about 15, or at least about 16 subunits. The pore is preferably a hexameric, heptameric, octameric or nonameric pore. The pore may be a homo-oligomer or a hetero-oligomer.

The transmembrane protein pore may comprise a barrel or channel through which the ions may flow. The subunits of the pore typically surround a central axis and contribute strands to a transmembrane β-barrel or channel or a transmembrane α-helix bundle or channel.

Typically, the barrel or channel of the transmembrane protein pore comprises amino acids that facilitate interaction with an analyte, such as a target polynucleotide (as described herein). These amino acids are preferably located near a constriction of the barrel or channel. The transmembrane protein pore typically comprises one or more positively charged amino acids, such as arginine, lysine or histidine, or aromatic amino acids, such as tyrosine or tryptophan. These amino acids typically facilitate the interaction between the pore and nucleotides, polynucleotides, or nucleic acids.

The nanopore may be a transmembrane protein pore derived from β-barrel pores or α-helix bundle pores. β-barrel pores comprise a barrel or channel that is formed from β-strands. Suitable β-barrel pores include, but are not limited to, β-toxins, such as α-hemolysin, anthrax toxin and leukocidins, and outer membrane proteins/porins of bacteria, such as Mycobacterium smegmatis porin (Msp), for example MspA, MspB, MspC or MspD, CsgG, outer membrane porin F (OmpF), outer membrane porin G (OmpG), outer membrane phospholipase A and Neisseria autotransporter lipoprotein (NaIP) and other pores, such as lysenin. α-helix bundle pores comprise a barrel or channel that is formed from α-helices. Suitable α-helix bundle pores include, but are not limited to, inner membrane proteins and a outer membrane proteins, such as WZA and ClyA toxin.

The nanopore may be a transmembrane pore derived from or based on Msp, α-hemolysin (α-HL), lysenin, CsgG, ClyA, Sp1 or haemolytic protein fragaceatoxin C (FraC).

The nanopore may be a transmembrane protein pore derived from CsgG, e.g., from CsgG from E. coli Str. K-12 substr. MC4100. Such a pore is oligomeric and typically comprises 7, 8, 9 or 10 monomers derived from CsgG. The pore may be a homo-oligomeric pore derived from CsgG comprising identical monomers. Alternatively, the pore may be a hetero-oligomeric pore derived from CsgG comprising at least one monomer that differs from the others. Examples of suitable pores derived from CsgG are disclosed in WO 2016/034591, WO 2017/149316, WO 2017/149317, WO 2017/149318, and WO 2019/002893 (all of which are incorporated herein by reference in their entireties).

The nanopore may be a transmembrane pore derived from lysenin. Examples of suitable pores derived from lysenin are disclosed in WO 2013/153359 (incorporated herein by reference in its entirety).

The nanopore may be a transmembrane pore derived from or based on α-hemolysin (α-HL). The wild type α-hemolysin pore is formed of 7 identical monomers or sub-units (i.e., it is heptameric). An α-hemolysin pore may be α-hemolysin-NN or a variant thereof. The variant preferably comprises N residues at positions E111 and K147.

The nanopore may be a transmembrane protein pore derived from Msp, e.g., from MspA. Examples of suitable pores derived from MspA are disclosed in WO 2012/107778 (incorporated herein by reference in its entirety).

The nanopore may be a transmembrane pore derived from or based on ClyA.

### Membrane

The detector or nanopore is typically present in a membrane. Any suitable membrane may be used.

The membrane is preferably an amphiphilic layer. An amphiphilic layer is a layer formed from amphiphilic molecules, such as phospholipids, which have both hydrophilic and lipophilic properties. The amphiphilic molecules may be synthetic or naturally occurring. Non-naturally occurring amphiphiles and amphiphiles which form a monolayer are known in the art and include, for example, block copolymers (Gonzalez-Perez et al., Langmuir, 2009, 25, 10447-10450). Block copolymers are polymeric materials in which two or more monomer sub-units that are polymerized together to create a single polymer chain. Block copolymers typically have properties that are contributed by each monomer sub-unit. However, a block copolymer may have unique properties that polymers formed from the individual sub-units do not possess. Block copolymers can be engineered such that one of the monomer sub-units is hydrophobic (i.e., lipophilic), whilst the other sub-unit(s) are hydrophilic whilst in aqueous media. In this case, the block copolymer may possess amphiphilic properties and may form a structure that mimics a biological membrane. The block copolymer may be a diblock (consisting of two monomer sub-units) but may also be constructed from more than two monomer sub-units to form more complex arrangements that behave as amphipiles. The copolymer may be a triblock, tetrablock or pentablock copolymer. The membrane may be a triblock copolymer membrane.

Archaebacterial bipolar tetraether lipids are naturally occurring lipids that are constructed such that the lipid forms a monolayer membrane. These lipids are generally found in extremophiles that survive in harsh biological environments, thermophiles, halophiles and acidophiles. Their stability is believed to derive from the fused nature of the final bilayer. It is straightforward to construct block copolymer materials that mimic these biological entities by creating a triblock polymer that has the general motif hydrophilic-hydrophobic-hydrophilic. This material may form monomeric membranes that behave similarly to lipid bilayers and encompass a range of phase behaviours from vesicles through to laminar membranes. Membranes formed from these triblock copolymers hold several advantages over biological lipid membranes. Because the triblock copolymer is synthesised, the exact construction can be carefully controlled to provide the correct chain lengths and properties required to form membranes and to interact with pores and other proteins.

Block copolymers may also be constructed from sub-units that are not classed as lipid sub-materials; for example, a hydrophobic polymer may be made from siloxane or other non-hydrocarbon-based monomers. The hydrophilic sub-section of block copolymer can also possess low protein binding properties, which allows the creation of a membrane that is highly resistant when exposed to raw biological samples. This head group unit may also be derived from non-classical lipid head-groups.

Triblock copolymer membranes also have increased mechanical and environmental stability compared with biological lipid membranes, for example a much higher operational temperature or pH range. The synthetic nature of the block copolymers provides a platform to customise polymer-based membranes for a wide range of applications.

The membrane may be one of the membranes disclosed in International Application No. WO2014/064443 or WO2014/064444 (both of which are incorporated herein by reference in their entireties).

The amphiphilic molecules may be chemically modified or functionalised to facilitate coupling of the polynucleotide. The amphiphilic layer may be a monolayer or a bilayer. The amphiphilic layer is typically planar. The amphiphilic layer may be curved. The amphiphilic layer may be supported.

Amphiphilic membranes are typically naturally mobile, essentially acting as two-dimensional fluids with lipid diffusion rates of approximately 10⁻⁸ cm s⁻¹. This means that the pore and coupled polynucleotide can typically move within an amphiphilic membrane.

The membrane may be a lipid bilayer. Lipid bilayers are models of cell membranes and serve as excellent platforms for a range of experimental studies. For example, lipid bilayers can be used for in vitro investigation of membrane proteins by single-channel recording. Alternatively, lipid bilayers can be used as biosensors to detect the presence of a range of substances. The lipid bilayer may be any lipid bilayer. Suitable lipid bilayers include, but are not limited to, a planar lipid bilayer, a supported bilayer, or a liposome. The lipid bilayer is preferably a planar lipid bilayer. Suitable lipid bilayers are disclosed in WO 2008/102121, WO 2009/077734, and WO 2006/100484 (incorporated herein by reference in their entireties).

Methods for forming lipid bilayers are known in the art. Lipid bilayers are commonly formed by the method of Montal and Mueller (Proc. Natl. Acad. Sci. USA., 1972; 69: 3561-3566).

A lipid bilayer may be formed as described in WO 2009/077734 (incorporated herein by reference in its entirety). In this method, the lipid bilayer is formed from dried lipids. A lipid bilayer may be formed across an opening as described in WO2009/077734.

The membrane may comprise a solid-state layer. Solid state layers can be formed from both organic and inorganic materials including, but not limited to, microelectronic materials, insulating materials such as Si₃N₄, Al₂O₃, and SiO, organic and inorganic polymers such as polyamide, plastics such as Teflon® or elastomers such as two-component addition-cure silicone rubber, and glasses. The solid-state layer may be formed from graphene. Suitable graphene layers are disclosed in WO 2009/035647 (incorporated herein by reference in its entirety). If the membrane comprises a solid-state layer, the pore is typically present in an amphiphilic membrane or layer contained within the solid-state layer, for instance within a hole, well, gap, channel, trench or slit within the solid-state layer. The skilled person can prepare suitable solid state/amphiphilic hybrid systems. Suitable systems are disclosed in WO 2009/020682 and WO 2012/005857 (incorporated herein by reference in their entireties). Any of the amphiphilic membranes or layers discussed above may be used.

The methods disclosed herein are typically carried out using (i) an artificial amphiphilic layer comprising a pore, (ii) an isolated, naturally occurring lipid bilayer comprising a pore, or (iii) a cell having a pore inserted therein. The methods are typically carried out using an artificial amphiphilic layer, such as an artificial triblock copolymer layer. The layer may comprise other transmembrane and/or intramembrane proteins as well as other molecules in addition to the pore. Suitable apparatus and conditions are discussed below. The method of the invention is typically carried out in vitro.

### Polynucleotide Binding Protein

As those skilled in the art will appreciate, any suitable polynucleotide binding protein can be used in the methods and products of the invention. The polynucleotide binding protein may be any protein that is capable of binding to a polynucleotide and controlling its movement with respect to a detector, e.g., a nanopore.

In more detail, polynucleotide binding proteins such as helicases can typically control the movement of DNA in at least two active modes of operation (when is provided with all the necessary components to facilitate movement e.g., ATP and Mg²⁺) and one inactive mode of operation (when not provided with the necessary components to facilitate movement; or when the polynucleotide binding protein is modified in order to prevent the active mode).

When provided with all the necessary components to facilitate movement, a polynucleotide binding protein may move along a polynucleotide such as DNA in either a 5′-3′ direction or a 3′-5′ direction. Many polynucleotide binding proteins process polynucleotides such as DNA in a 5′-3′ direction. Polynucleotide binding proteins which control the movement of polynucleotides in this manner are typically suitable for use in the method of the invention.

However, when a polynucleotide binding protein is not provided with the necessary components to facilitate movement or is modified in order to prevent it from actively controlling the movement of the polynucleotide with respect to the nanopore, it can still passively control the movement of the polynucleotide with respect to the nanopore. For example, the polynucleotide binding protein can bind to the polynucleotide and act as a brake slowing the movement of the polynucleotide when it is pulled into the pore by an applied field (e.g., by the first force in the method of the invention). In the “inactive” mode it typically does not matter whether the DNA is captured either 3′ or 5′ down (i.e., moves through the nanopore in a 5′-3′ direction or in a 3′-5′ direction), as the applied force provides the impetus to move the polynucleotide through the nanopore. However, in such embodiments, the polynucleotide binding protein may still control the movement of the polynucleotide with respect to the nanopore e.g., by acting as a brake. When in the inactive mode the movement control of a polynucleotide by a polynucleotide binding protein can be described in a number of ways including ratcheting, sliding, and braking. Typically the method of the invention do not comprise the use of a polynucleotide binding protein operating in the passive mode. However, when a polynucleotide binding protein the polynucleotide binding protein is used, it may be a polynucleotide binding protein operating in the passive mode.

Some methods of the invention may comprise use of a polynucleotide binding protein as a pausing moiety to impede the movement of the polynucleotide strand through the nanopore. The polynucleotide binding protein may be a protein which binds to polynucleotides but which does not have polynucleotide processing capacity, i.e., it is not a polynucleotide binding protein.

A polynucleotide-handling enzyme is a polypeptide that is capable of interacting with a polynucleotide. The enzyme may modify the polynucleotide by cleaving it to form individual nucleotides or shorter chains of nucleotides, such as di- or trinucleotides. The enzyme may modify the polynucleotide by orienting it or moving it to a specific position. A polynucleotide binding protein as used herein may be, or may be derived from a polynucleotide handling enzyme. A polynucleotide binding protein may be, or may be derived from a polynucleotide-handling enzyme.

The polynucleotide binding protein may be derived from a member of any of the Enzyme Classification (EC) groups 3.1.11, 3.1.13, 3.1.14, 3.1.15, 3.1.16, 3.1.21, 3.1.22, 3.1.25, 3.1.26, 3.1.27, 3.1.30 and 3.1.31.

Typically, the polynucleotide binding protein is a helicase, a polymerase, an exonuclease, a topoisomerase, or a variant thereof.

The polynucleotide binding protein may be modified to prevent the polynucleotide binding protein disengaging from the polynucleotide. Thus, the target polynucleotide preferably does not disengage from the polynucleotide binding protein.

As used herein, the term “disengaging” refers to the dissociation of the polynucleotide binding protein from the target polynucleotide. Thus, a polynucleotide binding protein may be modified to prevent it from dissociating from the target polynucleotide, e.g., into the reaction medium. It is important to distinguish potential “disengagement” of a polynucleotide binding protein from “unbinding” of a polynucleotide binding protein from a target polynucleotide. As used herein, “unbinding” refers to the transient release of the target polynucleotide the active site of the polynucleotide binding protein (described in more detail herein) but does not imply disengagement. Thus, for example, a polynucleotide binding protein may be modified to prevent the polynucleotide binding protein from disengaging from a polynucleotide, but without preventing the polynucleotide binding protein from unbinding from the polynucleotide. When unbound, the polynucleotide binding protein remains engaged with the target polynucleotide. For example, the polynucleotide binding protein may remain engaged with the target polynucleotide (i.e., it may be prevented from disengaging from the target polynucleotide) because it is topologically closed around the target polynucleotide. The polynucleotide binding site may remain free to bind or unbind the target polynucleotide such that the polynucleotide binding protein may bind or unbind to the target polynucleotide, whilst the polynucleotide binding protein remains engaged with the target polynucleotide. When the polynucleotide binding protein is unbound from the target polynucleotide it may be able to move on (e.g., along) the target polynucleotide under an applied force and may be capable of re-binding to the target polynucleotide. When engaged on the target polynucleotide but unbound from the target polynucleotide, the polynucleotide binding protein is not capable of dissociating from the target polynucleotide.

The polynucleotide binding protein can be adapted to prevent disengagement in any suitable way. For example, the polynucleotide binding protein can be loaded on the polynucleotide and then modified in order to prevent it from disengaging from the polynucleotide. Alternatively, the polynucleotide binding protein can be modified to prevent it from disengaging from the polynucleotide before it is loaded onto the polynucleotide. Modification of a polynucleotide binding protein and/or a polynucleotide binding protein in order to prevent it from disengaging from a polynucleotide can be achieved using methods known in the art, such as those discussed in WO 2014/013260, which is hereby incorporated by reference in its entirety, and with particular reference to passages describing the modification of polynucleotide binding proteins such as helicases in order to prevent them from disengaging with polynucleotide strands. For example, a polynucleotide binding protein can be modified by treating with tetramethylazodicarboxamide (TMAD). Various other closing moieties are described in WO 2021/255476 (incorporated herein by reference in its entirety).

For example, a polynucleotide binding protein and/or a polynucleotide binding protein may have a polynucleotide-unbinding opening, e.g., a cavity, cleft or void through which a polynucleotide strand may pass when the polynucleotide binding protein disengages from the strand. The polynucleotide-unbinding opening may be the opening through which a polynucleotide may pass when the polynucleotide binding protein disengages from the polynucleotide. The polynucleotide-unbinding opening for a given polynucleotide binding protein can be determined by reference to its structure, e.g., by reference to its X-ray crystal structure. The X-ray crystal structure may be obtained in the presence and/or the absence of a polynucleotide substrate. The location of a polynucleotide-unbinding opening in a given polynucleotide binding protein may be deduced or confirmed by molecular modelling using standard packages known in the art. The polynucleotide-unbinding opening may be transiently produced by movement of one or more parts e.g., one or more domains of the polynucleotide binding protein.

The polynucleotide binding protein may be modified by closing the polynucleotide-unbinding opening. The polynucleotide-unbinding opening may be closed with a closing moiety. Closing the polynucleotide-unbinding opening may therefore prevent the polynucleotide binding protein from disengaging from the polynucleotide. For example, the polynucleotide binding protein may be modified by covalently closing the polynucleotide-unbinding opening. However, as explained above closing the polynucleotide-unbinding opening does not necessarily prevent the target polynucleotide from unbinding from the polynucleotide binding site of the polynucleotide binding protein. A preferred protein for addressing in this way is a helicase.

The polynucleotide binding protein may be modified with a closing moiety for (i) topologically closing the polynucleotide binding site of the polynucleotide binding protein around the target polynucleotide and (ii) promoting unbinding of the target polynucleotide from the polynucleotide binding site of the polynucleotide binding protein and/or retarding re-binding of the target polynucleotide to the polynucleotide binding site of the polynucleotide binding protein. The polynucleotide binding protein may be modified in any suitable manner to facilitate attachment of such a closing moiety.

A closing moiety may comprise a bifunctional cross-linking moiety. The closing moiety may comprise a bifunctional cross-linker. The bifunctional crosslinker may attach at two points on the polynucleotide binding protein and close the polynucleotide-unbinding opening of the polynucleotide binding protein thereby preventing disengagement of the polynucleotide from the polynucleotide binding protein whilst allowing unbinding of the polynucleotide from the polynucleotide-binding site of the polynucleotide binding protein.

The closing moiety may attach at any suitable positions on the polynucleotide binding protein. For example, the closing moiety may crosslink two amino acid residues of the polynucleotide binding protein. Typically, at least one amino acid crosslinked by the closing moiety is a cysteine or a non-natural amino acid. The cysteine or non-natural amino acid may be introduced into the polynucleotide binding protein by substitution or modification of a naturally occurring amino acid residue of the polynucleotide binding protein. Methods for introducing non-natural amino acids are well known in the art and include for example native chemical ligation with synthetic polypeptide strands comprising such non-natural amino acids. Methods for introducing cysteines into a polynucleotide binding protein are likewise within the capability of one of skill in the art, for example using techniques disclosed in references such as Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016).

The closing moiety may have a length of from about 1 Å to about 100 Å. The length of the closing moiety may be calculated according to static bond lengths or more preferably using molecular dynamics simulations. The length may for example be from about 2 Å to about 80 Å, such as from about 5 Å to about 50 Å, e.g., from about 8 to about 30 Å such as from about 10 to about 25 Å or about 20 Å, e.g., about 1, 2, 3, 4, 5, 6, 7, 8, 9, 10, 11, 12, 13, 14, 15, 16, 17, 18, or 19 Å.

Polynucleotide binding proteins suitable for being closed using a closing moiety as described above are discussed in more detail herein. The polynucleotide binding protein is preferably a helicase, e.g., a Dda helicase as described herein.

The polynucleotide binding protein may be or may be derived from an exonuclease. Suitable enzymes include, but are not limited to, exonuclease I from E. coli, exonuclease III enzyme from E. coli, RecJ from T. thermophilus and bacteriophage lambda exonuclease, TatD exonuclease and variants thereof.

The polynucleotide binding protein may be a polymerase. The polymerase may be PyroPhage® 3173 DNA Polymerase (which is commercially available from Lucigen® Corporation), SD Polymerase (commercially available from Bioron®), Klenow from NEB or variants thereof. In one embodiment, the enzyme is Phi29 DNA polymerase or a variant thereof. Modified versions of Phi29 polymerase that may be used in the invention are disclosed in U.S. Pat. No. 5,576,204.

The polynucleotide binding protein may be a topoisomerase. In one embodiment, the topoisomerase is a member of any of the Moiety Classification (EC) groups 5.99.1.2 and 5.99.1.3. The topoisomerase may be a reverse transcriptase, which are enzymes capable of catalysing the formation of cDNA from a RNA template. They are commercially available from, for instance, New England Biolabs® and Invitrogen®.

The polynucleotide binding protein is preferably a helicase. Any suitable helicase can be used in accordance with the method of the invention. For example, the or each enzyme used in accordance with the present disclosure may be independently selected from a Hel308 helicase, a RecD helicase, a TraI helicase, a TrwC helicase, an XPD helicase, and a Dda helicase, or a variant thereof. Monomeric helicases may comprise several domains attached together. For instance, TraI helicases and TraI subgroup helicases may contain two RecD helicase domains, a relaxase domain and a C-terminal domain. The domains typically form a monomeric helicase that is capable of functioning without forming oligomers. Particular examples of suitable helicases include Hel308, NS3, Dda, UvrD, Rep, PcrA, Pif1 and TraI. These helicases typically work on single stranded DNA. Examples of helicases that can move along both strands of a double stranded DNA include FtfK and hexameric enzyme complexes, or multisubunit complexes such as RecBCD. The polynucleotide binding protein is preferably a Dda (DNA-dependent ATPase) helicase.

Hel308 helicases are described in publications such as WO 2013/057495, the entire contents of which are incorporated by reference. RecD helicases are described in publications such as WO 2013/098562, the entire contents of which are incorporated by reference. XPD helicases are described in publications such as WO 2013/098561, the entire contents of which are incorporated by reference. Dda helicases are described in publications such as WO 2015/055981 and WO 2016/055777, the entire contents of each of which are incorporated by reference.

The helicase may be Trwc Cba or a variant thereof, Hel308 Mbu or a variant thereof or Dda or a variant thereof. Variants may differ from the native sequences in any of the ways discussed herein. An example variant of Dda comprises E94C/A360C. A further example variant of Dda comprises E94C/A360C and then (ΔM1)G1G2 (i.e., deletion of M1 and then addition of G1 and G2).

### Disclaimers

The 3′ end of the telomere adaptor preferably does not comprise one or more universal nucleotides. A universal nucleotide is one which will hybridise or bind to some degree to all of the nucleotides in the template polynucleotide. A universal nucleotide is preferably one which will hybridise or bind to some degree to nucleotides comprising the nucleosides adenosine (A), thymine (T), uracil (U), guanine (G) and cytosine (C). The universal nucleotide may hybridise or bind more strongly to some nucleotides than to others. For instance, a universal nucleotide (I) comprising the nucleoside, 2′-deoxyinosine, will show a preferential order of pairing of I-C>I-A>I-G approximately=I-T. The polymerase will replace a nucleotide species with a universal nucleotide if the universal nucleotide takes the place of the nucleotide species in the population. For instance, the polymerase will replace dGMP with a universal nucleotide, if it is contacted with a population of free dAMP, dTMP, dCMP and the universal nucleotide.

The universal nucleotide preferably comprises one of the following nucleobases: hypoxanthine, 4-nitroindole, 5-nitroindole, 6-nitroindole, formylindole, 3-nitropyrrole, nitroimidazole, 4-nitropyrazole, 4-nitrobenzimidazole, 5-nitroindazole, 4-aminobenzimidazole or phenyl (C6-aromatic ring). The universal nucleotide more preferably comprises one of the following nucleosides: 2′-deoxyinosine, inosine, 7-deaza-2′-deoxyinosine, 7-deaza-inosine, 2-aza-deoxyinosine, 2-aza-inosine, 2-O′-methylinosine, 4-nitroindole 2′-deoxyribonucleoside, 4-nitroindole ribonucleoside, 5-nitroindole 2′-deoxyribonucleoside, 5-nitroindole ribonucleoside, 6-nitroindole 2′-deoxyribonucleoside, 6-nitroindole ribonucleoside, 3-nitropyrrole 2′-deoxyribonucleoside, 3-nitropyrrole ribonucleoside, an acyclic sugar analogue of hypoxanthine, nitroimidazole 2′-deoxyribonucleoside, nitroimidazole ribonucleoside, 4-nitropyrazole 2′-deoxyribonucleoside, 4-nitropyrazole ribonucleoside, 4-nitrobenzimidazole 2′-deoxyribonucleoside, 4-nitrobenzimidazole ribonucleoside, 5-nitroindazole 2′-deoxyribonucleoside, 5-nitroindazole ribonucleoside, 4-aminobenzimidazole 2′-deoxyribonucleoside, 4-aminobenzimidazole ribonucleoside, phenyl C-ribonucleoside, phenyl C-2′-deoxyribosyl nucleoside, 2′-deoxynebularine, 2′-deoxyisoguanosine, K-2′-deoxyribose, P-2′-deoxyribose and pyrrolidine. The universal nucleotide more preferably comprises 2′-deoxyinosine. The universal nucleotide is more preferably IMP or dIMP. The universal nucleotide is most preferably dPMP (2′-Deoxy-P-nucleoside monophosphate) or dKMP (N6-methoxy-2, 6-diaminopurine monophosphate).

The method of the invention preferably does not comprise restriction digestion. The method of the invention preferably does not comprise amplifying the at least part of a telomere or polymerase chain reaction (PCR). The method of the invention preferably does not comprise (i) restriction digestion and/or (ii) amplifying the at least part of a telomere or polymerase chain reaction (PCR). The method of the invention preferably does not comprise (i) restriction digestion, (ii) amplifying the at least part of a telomere and (iii) or polymerase chain reaction (PCR). These embodiments provide the advantages discussed above.

### Telomere Source

The method of the invention may be carried out on any suitable sample. The sample is typically one that is known to contain or is suspected of containing at least part of a telomere.

The sample may be a biological sample. The invention may be carried out in vitro on a sample obtained from or extracted from any organism or microorganism. Telomeres are typically only present in eukaryotes. The at least part of a telomere may be from any eukaryote, including any of those listed below.

The sample is preferably a fluid sample. The sample typically comprises a body fluid. The body fluid may be obtained from a human or animal. The human or animal may have, be suspected of having or be at risk of a disease. The sample may be urine, lymph, saliva, mucus, seminal fluid, or amniotic fluid, but is preferably whole blood, plasma, or serum. Typically, the sample is human in origin, but alternatively it may be from another mammal such as from commercially farmed animals such as horses, cattle, sheep, or pigs or may alternatively be pets such as cats or dogs.

Alternatively, a sample of plant origin is typically obtained from a commercial crop, such as a cereal, legume, fruit, or vegetable, for example wheat, barley, oats, canola, maize, soya, rice, bananas, apples, tomatoes, potatoes, grapes, tobacco, beans, lentils, sugar cane, cocoa, cotton, tea, or coffee.

The sample may be a non-biological sample. The non-biological sample is preferably a fluid sample. Examples of non-biological samples include surgical fluids, water such as drinking water, sea water or river water, and reagents for laboratory tests.

The sample may be processed prior to being assayed, for example by centrifugation or by passage through a membrane that filters out unwanted molecules or cells, such as red blood cells. The sample may be measured immediately upon being taken. The sample may also be typically stored prior to assay, preferably below −70° C.

The sample typically comprises genomic DNA. The sample may comprise T-cell DNA.

The at least part of a telomere may be from common organisms such as plants, or animals. The at least part of a telomere is often obtained from a human or animal, e.g., from urine, lymph, saliva, mucus, seminal fluid, or amniotic fluid, or from whole blood, plasma, or serum. The at least part of a telomere may be obtained from a plant e.g., a cereal, legume, fruit, or vegetable.

### GENERAL METHODS

As mentioned above, the method of the invention may be operated using any suitable detector, and as such any suitable apparatus for detecting polynucleotides can be used.

The method of the invention may be carried out using any apparatus that is suitable for nanopore sensing. For example, the apparatus may comprise a chamber comprising an aqueous solution and a barrier that separates the chamber into two sections. The barrier may have an aperture in which a membrane containing a transmembrane pore is formed. Transmembrane pores are described herein.

The methods may be carried out using the apparatus described in WO 2008/102120, WO 2010/122293, or WO 00/28312 (incorporated herein by reference in their entireties). In brief. the binding of a molecule (e.g., a target polynucleotide) in the channel of a pore will have an effect on the open-channel ion flow through the pore, which is the essence of “molecular sensing” of pore channels. Variation in the open-channel ion flow can be measured using suitable measurement techniques by the change in electrical current. The degree of reduction in ion flow, as measured by the reduction in electrical current, is related to the size of the obstruction within, or in the vicinity of, the pore. Binding of a molecule of interest (e.g., the target polynucleotide) in or near the pore therefore provides a detectable and measurable event, thereby forming the basis of a “biological sensor”. Detecting the presence of biological molecules finds application in personalised drug development, medicine, diagnostics, life science research, environmental monitoring and in the security and/or the defence industry.

When used to characterize the polynucleotide, the presence, absence or one or more characteristics of the target polynucleotide are determined. The methods may be for determining the presence, absence or one or more characteristics of at least one target polynucleotide. The methods may concern determining the presence, absence or one or more characteristics of two or more target polynucleotide. The methods may comprise determining the presence, absence or one or more characteristics of any number of target polynucleotides, such as 2, 5, 10, 15, 20, 30, 40, 50, 100 or more target polynucleotides. Any number of characteristics of the one or more target polynucleotides may be determined, such as 1, 2, 3, 4, 5, 10 or more characteristics. Characteristics amenable to being detected in the methods provide herein include the identity or sequence of the polynucleotide, the length, of the polynucleotide, whether or not the polynucleotide is modified, etc. In some embodiments the method of the invention are methods of sequencing the at least part of the telomere. In some embodiments the sequence of the at least part of the telomere may be determined in real-time by aligning real-time signal or basecalling to known references. Exemplary methods of determining a polynucleotide sequence are described in WO 2016/059427 (incorporated herein by reference in its entirety).

When used to characterize the polynucleotide, the methods may involve measuring the ion current flow through the pore, typically by measurement of a current. Alternatively, the ion flow through the pore may be measured optically, such as disclosed by Heron et al: J. Am. Chem. Soc. 9 Vol. 131, No. 5, 2009. Therefore, the apparatus may also comprise an electrical circuit capable of applying a potential and measuring an electrical signal across the membrane and pore. The characterisation methods may be carried out using a patch clamp or a voltage clamp. The characterisation methods preferably involve the use of a voltage clamp.

The methods may involve measuring an optical signal as described in Chen et al, Nature Communications (2018)9:1733, the entire contents of which are hereby incorporated by reference. For example, a nanopore such as an optically engineered nanopore structure (e.g., a plasmonic nanoslit) may be used to locally enable single-molecule surface enhanced Raman spectroscopy (SERS) to allow the characterisation of the polynucleotide through direct Raman spectroscopic detection.

The methods may be carried out on a silicon-based array of wells where each array comprises 128, 256, 512, 1024, 2000, 3000, 4000, 6000, 10000, 12000, 15000 or more wells.

The methods may involve the measuring of a current flowing through the pore. The method is typically carried out with a voltage applied across the membrane and pore. The voltage used is typically from +2 V to −2 V, typically −400 mV to +400 mV. The voltage used is preferably in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage used is more preferably in the range 100 mV to 240 mV and most preferably in the range of 120 mV to 220 mV. It is possible to increase discrimination between different nucleotides by a pore by using an increased applied potential.

The methods are typically carried out in the presence of any charge carriers, such as metal salts, for example alkali metal salts, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or 1-ethyl-3-methyl imidazolium chloride. In the exemplary apparatus discussed above, the salt is present in the aqueous solution in the chamber. Potassium chloride (KCl), sodium chloride (NaCl) or caesium chloride (CsCl) is typically used. KCl is preferred. The salt may be an alkaline earth metal salt such as calcium chloride (CaCl2). The salt concentration may be at saturation. The salt concentration may be 3M or lower and is typically from 0.1 to 2.5 M, from 0.3 to 1.9 M, from 0.5 to 1.8 M, from 0.7 to 1.7 M, from 0.9 to 1.6 M or from 1 M to 1.4 M. The salt concentration is preferably from 150 mM to 1 M. The method is preferably carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. High salt concentrations provide a high signal to noise ratio and allow for currents indicative of binding/no binding to be identified against the background of normal current fluctuations.

The methods are typically carried out in the presence of a buffer. In the exemplary apparatus discussed above, the buffer is present in the aqueous solution in the chamber. Any suitable buffer may be used. Typically, the buffer is HEPES. Another suitable buffer is Tris-HCl buffer. The methods are typically carried out at a pH of from 4.0 to 12.0, from 4.5 to 10.0, from 5.0 to 9.0, from 5.5 to 8.8, from 6.0 to 8.7 or from 7.0 to 8.8 or 7.5 to 8.5. The pH used is preferably about 7.5.

The methods may be carried out at from 0° C. to 100° C., from 15° C. to 95° C., from 16° C. to 90° C., from 17° C. to 85° C., from 18° C. to 80° C., 19° C. to 70° C., or from 20° C. to 60° C. The methods are typically carried out at room temperature. The methods are optionally carried out at a temperature that supports enzyme function, such as about 37° C.

Any of the proteins described herein, such as the protein pores, may be made synthetically or by recombinant means. For example, the pore may be synthesised by in vitro translation and transcription (IVTT). The amino acid sequence of the pore may be modified to include non-naturally occurring amino acids or to increase the stability of the protein. When a protein is produced by synthetic means, such amino acids may be introduced during production. The pore may also be altered following either synthetic or recombinant production.

Any of the proteins described herein, such as the protein pores, can be produced using standard methods known in the art. Polynucleotide sequences encoding a pore or construct may be derived and replicated using standard methods in the art. Polynucleotide sequences encoding a pore or construct may be expressed in a bacterial host cell using standard techniques in the art. The pore may be produced in a cell by in situ expression of the polypeptide from a recombinant expression vector. The expression vector optionally carries an inducible promoter to control the expression of the polypeptide. These methods are described in Sambrook, J. and Russell, D. (2001). Molecular Cloning: A Laboratory Manual, 3rd Edition. Cold Spring Harbor Laboratory Press, Cold Spring Harbor, NY.

The pore may be produced in large scale following purification by any protein liquid chromatography system from protein producing organisms or after recombinant expression. Typical protein liquid chromatography systems include FPLC, AKTA systems, the Bio-Cad system, the Bio-Rad BioLogic system, and the Gilson HPLC system.

### Polynucleotide Telomere Adaptors

The invention also provides a polynucleotide telomere adaptor wherein the 3′ end of the adaptor specifically hybridises to the first part of the overhanging strand at the end of a telomere and the 5′ end of the adaptor is not complementary to the opposite part of the overhanging strand at the end of the telomere. The telomere adaptor may be any of those defined above with reference to the method of the invention, including an extended telomere adaptor. The telomere adaptor may further comprise a splint polynucleotide and/or a sequencing adaptor.

The invention also provides a population of six telomere adaptors each of which has a 3′ end which specifically hybridises to one of the six possible sequences of the first part of the overhanging strand at the end of a telomere and a 5′ end which is not complementary to the opposite part of the overhanging strand at the end of the telomere. The population may be any of those defined above with reference to the method of the invention, including extended telomere adaptors.

The population preferably comprises six telomere adaptors which comprise or consist of the following sequences:

(SEQ ID NO: 1)
AGCAATACGTAACTGAACGAAGTACCCTAA;
(SEQ ID NO: 2)
AGCAATACGTAACTGAACGAAGTAACCCTA;
(SEQ ID NO: 3)
AGCAATACGTAACTGAACGAAGTTAACCCT;
(SEQ ID NO: 4)
AGCAATACGTAACTGAACGAAGTCTAACCC;
(SEQ ID NO: 5)
AGCAATACGTAACTGAACGAAGTCCTAACC;
and
(SEQ ID NO: 6)
AGCAATACGTAACTGAACGAAGTCCCTAAC.

These are shown in Table 3 of Example 1 and FIG. 1.

The population preferably comprises six telomere adaptors which comprise or consist of the following sequences:

(SEQ ID NO: 14)
AGCAATACGTAACTGAACGAAGTCCCTAACCCTAACCCTAA;
(SEQ ID NO: 15)
AGCAATACGTAACTGAACGAAGTTAACCCTAACCCTAACCC;
(SEQ ID NO: 16)
AGCAATACGTAACTGAACGAAGTCTAACCCTAACCCTAACC;
(SEQ ID NO: 17)
AGCAATACGTAACTGAACGAAGTCCTAACCCTAACCCTAAC;
(SEQ ID NO: 18)
AGCAATACGTAACTGAACGAAGTAACCCTAACCCTAACCCT;
and
(SEQ ID NO: 19)
AGCAATACGTAACTGAACGAAGTACCCTAACCCTAACCCTA.

These are shown in Table 14.

The population preferably comprises six extended telomere adaptors which comprise or consist of the following sequences:

SEQ ID NO: 11 covalently attached to
(SEQ ID NO: 1)
AGCAATACGTAACTGAACGAAGTACCCTAA;
SEQ ID NO: 11 covalently attached to
(SEQ ID NO: 2)
AGCAATACGTAACTGAACGAAGTAACCCTA;
SEQ ID NO: 11 covalently attached to
(SEQ ID NO: 3)
AGCAATACGTAACTGAACGAAGTTAACCCT;
SEQ ID NO: 11 covalently attached to
(SEQ ID NO: 4)
AGCAATACGTAACTGAACGAAGTCTAACCC;
SEQ ID NO: 11 covalently attached to
(SEQ ID NO: 5)
AGCAATACGTAACTGAACGAAGTCCTAACC;
and
SEQ ID NO: 11 covalently attached to
(SEQ ID NO: 6)
AGCAATACGTAACTGAACGAAGTCCCTAAC.

The population preferably comprises six extended telomere adaptors which comprise or consist of the following sequences:

(SEQ ID NO: 25)
GCTTGGGTGTTTAACCNNNNAGCAATACGTAACTGAACGAAGTACCCT
AA;
(SEQ ID NO: 26)
GCTTGGGTGTTTAACCNNNNAGCAATACGTAACTGAACGAAGTAACCC
TA;
(SEQ ID NO: 27)
GCTTGGGTGTTTAACCNNNNAGCAATACGTAACTGAACGAAGTTAACC
CT;
(SEQ ID NO: 28)
GCTTGGGTGTTTAACCNNNNAGCAATACGTAACTGAACGAAGTCTAAC
CC;
(SEQ ID NO: 29)
GCTTGGGTGTTTAACCNNNNAGCAATACGTAACTGAACGAAGTCCTAA
CC;
and
(SEQ ID NO: 30)
GCTTGGGTGTTTAACCNNNNAGCAATACGTAACTGAACGAAGTCCCTA
AC,
wherein N is Int 5-Nitroindole (i5NiTInd).

The population preferably comprises six extended telomere adaptors which comprise or consist of the following sequences:

SEQ ID NO: 11 covalently attached to
(SEQ ID NO: 14)
AGCAATACGTAACTGAACGAAGTCCCTAACCCTAACCCTAA;
SEQ ID NO: 11 covalently attached to
(SEQ ID NO: 15)
AGCAATACGTAACTGAACGAAGTTAACCCTAACCCTAACCC;
SEQ ID NO: 11 covalently attached to
(SEQ ID NO: 16)
AGCAATACGTAACTGAACGAAGTCTAACCCTAACCCTAACC;
SEQ ID NO: 11 covalently attached to
(SEQ ID NO: 17)
AGCAATACGTAACTGAACGAAGTCCTAACCCTAACCCTAAC;
SEQ ID NO: 11 covalently attached to
(SEQ ID NO: 18)
AGCAATACGTAACTGAACGAAGTAACCCTAACCCTAACCCT;
and
SEQ ID NO: 11 covalently attached to
(SEQ ID NO: 19)
AGCAATACGTAACTGAACGAAGTACCCTAACCCTAACCCTA.

The two sequences in the extended adaptors are preferably covalently attached by one or more, such as two or more, three or more or four or more, universal nucleotides, such as Int 5-Nitroindole (i5NiTInd). The two sequences in the extended adaptors are more preferably covalently attached by 4 i5NiTInds. The extended telomere adaptors preferably comprise a click chemistry group, such as DBCOTEG, at their 5′ ends. Examples of these extended adaptors are shown in Table 7 and FIG. 4.

The telomere adaptors in the population may each further comprise a splint polynucleotide and/or a sequencing adaptor.

### Kits and Systems

The invention also provides a kit for characterising at least part of a telomere, comprising (a) one or more polynucleotide telomere adaptors of the invention or a population of six telomere adaptors of the invention and (b) one or more splint polynucleotides or one or more polynucleotide extensions. The one or more telomere adaptors or the population of six telomere adaptors may be any of those defined above with reference to the method of the invention. The one or more splint polynucleotides or the one or more polynucleotide extensions may be any of those defined above with reference to the method of the invention. The one or more telomere adaptors and the one or more polynucleotide extensions preferably comprise click chemistry groups.

The kit of the invention preferably further comprises one or more sequencing adaptors. The one or more sequencing adaptors may be any of those discussed above with reference to the method of the invention.

The kit preferably further comprises a polymer-guided effector protein and one or more guide polymers. The polymer-guided effector protein and the one or more guide polymers may be any of those discussed above with reference to the method of the invention. The polymer-guided effector protein is preferably a Cas9 protein. The one or more guide polymers are preferably one or more guide RNAs.

The invention also provides a system for conducting the method of the invention. The system is for characterising at least part of a telomere. The system comprises (a) one or more polynucleotide telomere adaptors of the invention or a population of six telomere adaptors of the invention and (b) a nanopore. Any of the embodiments discussed above with reference to the method of the invention equally apply to the system of the invention.

The nanopore is preferably present in a membrane. Suitable membranes are discussed above. The system may comprise any of the membranes disclosed above, such as an amphiphilic layer, a triblock copolymer membrane or a solid-state layer. The membrane is typically part of an array of membranes, wherein each membrane preferably comprises a nanopore. The array may be any of those described in WO 2018/060740 (incorporated herein by reference in its entirety).

The system is preferably adapted to apply a voltage across the membrane and to take one or more electrical measurements. Suitable adaptations are discussed in WO 2018/060740 (incorporated herein by reference in its entirety).

The system may further comprise a polynucleotide binding protein. The kit may further comprise a microparticle. Any of the embodiments discussed above with reference to the method of the invention equally apply to the system of the invention.

The system may further comprise one or more splint polynucleotides, one or more polynucleotide extensions and/or one or more sequencing adaptors. Any of the embodiments discussed above with reference to the method of the invention equally apply to the system of the invention.

The system or kit may additionally comprise one or more other reagents or instruments which enable any of the embodiments mentioned above to be carried out. Such reagents or instruments include one or more of the following: suitable buffer(s) (aqueous solutions), means to obtain a sample from a subject (such as a vessel or an instrument comprising a needle), means to amplify and/or express polynucleotides, a membrane as defined above or voltage or patch clamp apparatus. Reagents may be present in the system or kit in a dry state such that a fluid sample is used to resuspend the reagents. The system or kit may also, optionally, comprise instructions to enable the system or kit to be used in the methods described herein or details regarding for which organism the method may be used. The system or kit may comprise a magnet or an electromagnet. The system or kit may, optionally, comprise nucleotides.

The following Examples illustrate the invention. It is to be understood that although particular embodiments, specific configurations as well as materials and/or molecules, have been discussed herein for methods according to the invention, various changes or modifications in form and detail may be made without departing from the scope and spirit of this invention. The following examples are provided to better illustrate particular embodiments, and they should not be considered limiting the application. The application is limited only by the claims.

### Example 1

This Example demonstrates the assembly of a telomere adaptor as described herein, and its use in combination with a sequencing adaptor for nanopore sequencing. The sequencing adapter is attached via a ligation reaction.

Assembly and Ligation of Telomere Adaptors onto Chromosome Ends

Telomere adaptors were assembled using the following sequences in Table 3 (as shown in FIG. 1).

TABLE 3
T1 to T6 telomere adaptors used in Example 1:

| | Sequences | SEQ ID NO: |
|---|---|---|
| T1 | /5Phos/AGCAATACGTAACTGAACGAAGTACCCTAA | 1 |
| T2 | /5Phos/AGCAATACGTAACTGAACGAAGTAACCCTA | 2 |
| T3 | /5Phos/AGCAATACGTAACTGAACGAAGTTAACCCT | 3 |
| T4 | /5Phos/AGCAATACGTAACTGAACGAAGTCTAACCC | 4 |
| T5 | /5Phos/AGCAATACGTAACTGAACGAAGTCCTAACC | 5 |
| T6 | /5Phos/AGCAATACGTAACTGAACGAAGTCCCTAAC | 6 |

The underlined bases hybridize with the G-rich strand.
1) 1 μM T1 through T6 was mixed in equal volumes to make “Telo-Mix”.
2) The ligation reaction was assembled in Table 4 below:

| Reagent | Volume | | Final concentration | |
|---|---|---|---|---|
| rCutSmart Buffer (10x) | 20 | μL | 1 | X |
| 100 mM ATP | 2 | μL | 1 | mM |
| gDNA (unsheared) | 5 | μg | 25 | ng/μL |
| Telo-Mix (1 μM) | 20 | μL | 100 | nM |
| NEB T4 DNA ligase (2000 U/μL) | 5 | μL | 50 | U/μL |

| | |
|---|---|
| H₂O | To 200 μL |
| Final volume | 200 μL |

3) Incubation was carried out at 35° C. for 16 hours, followed by heat inactivation of T4 at 65° C. for 10 min.

The protocol includes an optional step of digesting adaptor ligated DNA:
1) 4 μL EcoRI added to the 200 μL ligation reaction.
2) Incubation carried out at 37° C. for 15 min.

### Annealing of the “Splint” Oligo

Annealing of the splint oligo was carried out as follows (also shown in FIG. 2).
1) The above assembly reaction was cleaned up with 0.4×SPRI beads.
2) DNA was eluted in 190 μL DI water (37° C., 15 min, occasional mixing).
3) 10 μL NaCl (1 M) solution was added to give a final NaCl concentration of 50 mM.
4) 2 μL 10 μM “splint” oligo was added to give a final “splint” concentration of 100 nM.

TABLE 5

| | | SEQ |
|---|---|---|
| Splint | Sequences | ID NO: |
| S1 | ACTTCGTTCAGTTACGTATTGCTAGCAAT | 9 |
| S2 | ACTTCGTTCAGTTACGTATTGCTA | 10 |

The underlined bases can hybridise with a nanopore sequencing adapter.
5) Incubation was carried out at 50° C. for 1 hour.
6) The reaction was cleaned up with 0.4×SPRI beads.
7) Elution in 30 μL water. (37° C., 15 min, occasional mixing).

### Ligation of Nanopore Sequencing Adapters

1) The sequencing adaptor ligation reaction was assembled as below using Oxford Nanopore Technologies' Adapter Mix II Expansion kit (FIG. 3):

TABLE 6

| Reagent | Volume |
|---|---|
| DNA in water | 30 μL |
| AMII-H adaptor | 5 μL |
| NEBNext Quick ligation 5x buffer | 10 μL |
| Quick T4 DNA ligase | 5 μL |
| Total | 50 μL |

2) Incubation was carried out at room temperature for 20 min.
3) The reaction was cleaned up with 20 μL AXP beads and LFB.
4) Elution in 15 μL Elution Buffer (37° C., 15 min).

Nanopore sequencing was carried out using a GridION device from Oxford Nanopore Technologies fitted with FLO-MIN106 flow cells.

### Example 2

This Example demonstrates the assembly of a telomere adaptor as described herein, and its use in combination with a sequencing adaptor for nanopore sequencing. The sequencing adapter is attached via a “click” chemistry reaction.

Telomere adaptors were assembled using the following sequences (FIG. 4)
1) Click telomere adaptors all having 5′ DBCOTEG (Table 7):

| | Sequences | SEQ ID NOS: |
|---|---|---|
| T1 | /5DBCOTEG/GCTTGGGTGTTTAACC/i5NITInd/i5NiTInd | 11 and 1 linked by 4x |
| | /i5NITInd/i5NiTInd/ | i5NiTInd (full sequence in |
| | AGCAATACGTAACTGAACGAAGTACCCTAA | SEQ ID NO: 25) |
| T2 | /5DBCOTEG/GCTTGGGTGTTTAACC/i5NiTInd/i5NiTInd | 11 and 2 linked by 4x |
| | /i5NiTInd/i5NiTInd/ | i5NiTInd (full sequence in |
| | AGCAATACGTAACTGAACGAAGTAACCCTA | SEQ ID NO: 26) |
| T3 | /5DBCOTEG/GCTTGGGTGTTTAACC/i5NITInd/i5NiTInd | 11 and 3 linked by 4x |
| | /i5NiTInd/i5NiTInd/ | i5NiTInd (full sequence in |
| | AGCAATACGTAACTGAACGAAGTTAACCCT | SEQ ID NO: 27) |
| T4 | /5DBCOTEG/GCTTGGGTGTTTAACC/i5NITInd/i5NiTInd | 11 and 4 linked by 4x |
| | /i5NiTInd/i5NiTInd/ | i5NiTInd (full sequence in |
| | AGCAATACGTAACTGAACGAAGTCTAACCC | SEQ ID NO: 28) |
| T5 | /5DBCOTEG/GCTTGGGTGTTTAACC/i5NITInd/i5NiTInd | 11 and 5 linked by 4x |
| | /i5NiTInd/i5NiTInd/ | i5NiTInd (full sequence in |
| | AGCAATACGTAACTGAACGAAGTCCTAACC | SEQ ID NO: 29) |
| T6 | /5DBCOTEG/GCTTGGGTGTTTAACC/i5NITInd/i5NiTInd | 11 and 6 linked by 4x |
| | /i5NITInd/i5NiTInd/ | i5NiTInd (full sequence in |
| | AGCAATACGTAACTGAACGAAGTCCCTAAC | SEQ ID NO: 30) |

The underlined bases hybridize with the G-rich strand.
2) 1 μM T1 through T6 was mixed in equal volumes to make “Telo-Mix”.
3) The ligation reaction was assembled as below (Table 8):

| Reagent | Volume | | Final concentration | |
|---|---|---|---|---|
| rCutSmart Buffer (10x) | 20 | μL | 1 | X |
| 100 mM ATP | 2 | μL | 1 | mM |
| gDNA (unsheared) | 5 | μg | 25 | ng/μL |
| Telo-Mix (1 μM) | 20 | μL | 100 | nM |
| NEB T4 DNA ligase (2000 U/μL) | 5 | μL | 50 | U/μL |

| | |
|---|---|
| H₂O | To 200 μL |
| Final volume | 200 μL |

4) Incubation was carried out at 35° C. for 16 hours, followed by heat inactivation of T4 at 65° C. for 10 min.
5) 1 μL NEB Exonuclease I was added to the reaction.
6) Incubation was carried out at 37° C. for 15 min, followed by 80° C. for 15 min.

The protocol may include the optional step of digesting “Telo Mix” ligated DNA:
1) 4 μL EcoRI added to the 200 μL ligation reaction.
2) Incubation carried out at 37° C. for 15 min.

### Addition of “Click” Nanopore Sequencing Adapters

1) The above reaction was cleaned up with 0.4×SPRI beads.
2) Elution in 12 μL Elution Buffer. (37° C., 15 min)
3) 1 μL of sequencing adapter RAP-T from Oxford Nanopore Technologies' kit SQK-PCS111 was added.
4) Incubation was carried out at room temperature for 10 min.

Nanopore sequencing was carried out using a GridION device from Oxford Nanopore Technologies fitted with FLO-MIN106 flow cells.

### Example 3

This Example demonstrates the assembly of a biotinylated telomere adaptor as described herein, and its use in combination with a sequencing adaptor for nanopore sequencing. The sequencing adapter is attached via a ligation reaction.

Biotinylated telomere adaptors can be annealed onto chromosome ends, allowing sequencing of both strands.

Biotinylated telomere adaptors were assembled using the following sequences (as shown in FIG. 6).
1) Biotinylated telomere adaptors (Table 9):

| | Sequences | SEQ ID NO: |
|---|---|---|
| T1 | /BIOTIN/AGCAATACGTAACTGAACGAAGTCCCTAACCCTAACCCTAA | 12 |
| T2 | /BIOTIN/AGCAATACGTAACTGAACGAAGTCTAACCCTAACCCTAACC | 13 |

The underlined bases hybridize with the G-rich strand.
1) 1 μM T1 and T2 were mixed in equal volumes to make “Telo-Mix”.
2) The annealing reaction was assembled as below (Table 10):

| Reagent | Volume | | Final concentration | |
|---|---|---|---|---|
| rCutSmart Buffer (10x) | 20 | μL | 1 | X |
| gDNA (unsheared) | 5 | μg | 25 | ng/μL |
| Telo-Mix (1 μM) | 20 | μL | 100 | nM |

| | |
|---|---|
| H₂O | To 200 μL |
| Final volume | 200 μL |

3) Incubation was carried out at 35° C. for 16 hours.
4) A streptavidin Dynabeads® biotinylated pull-down protocol was followed. The telomere strands were eluted into 48 μL of water by heating the beads at 50° C.

At this point, the telomeric reads of interest are enriched in the supernatant and the telomeric adapters have attached to the beads, separated from the free-floating strands with telomeric overhangs. The protocol now moves forward with the supernatant, following the ONT “Genomic DNA By Ligation (SQK-LSK110)” protocol.
5) The telomeric overhang was filled in using NEBNext® FFPE DNA Repair Mix and NEBNext® Ultra™ II End Repair/dA-Tailing Module (Table 11):

| Reagent | Volume | |
|---|---|---|
| Telomere Enriched DNA | 47 | μL |
| NEBNext FFPE DNA Repair Buffer | 3.5 | μL |
| NEBNext FFPE DNA Repair Mix | 2 | μg |
| Ultra II End-Prep Reaction Buffer | 3.5 | μL |
| Ultra II End-Prep Enzyme Mix | 3 | μL |
| Final volume | 60 | μL |

Using a thermal cycler, the reaction was incubated at 20° C. for 5 minutes and 65° C. for 5 minutes.
6) The above reaction was cleaned up with 1×SPRI beads and 70% ethanol.
7) The DNA was eluted in 61 μL DI water (Room temperature, 10 min, occasional mixing).
8) The sequencing adaptor ligation reaction was assembled as below using sequencing adaptors from Oxford Nanopore Technologies' LSK109-112 kit (Table 12):

| Reagent | Volume | |
|---|---|---|
| DNA in water | 60 | μL |
| Adapter Mix F (AMX-F) | 5 | μL |
| Ligation Buffer (LNB) | 25 | μL |
| Quick T4 DNA ligase | 10 | μL |
| Total | 100 | μL |

9) The reaction was incubated at room temperature for 10 min.
10) The reaction was cleaned up with 40 μL AXP beads and LFB.
11) Elution in 15 μL Elution Buffer (37° C., 15 min).

Ends were dA-tailed and ONT sequencing adapters attached by ligation. Sequencing was carried out using a GridION device from Oxford Nanopore Technologies fitted with FLO-MIN106 flow cells.

### Example 4

This Example demonstrate the analysis of data obtained from the experiment described in Example 1.

Data output in the Fast5 file format were basecalled using the Bonito basecaller* (Oxford Nanopore Technologies) with a telomere trained model. *https://github.com/nanoporetech/bonito.

Potential telomeric reads were identified in base space using noise cancelling repeat finder, minimum motif repeat length of 500 bp. Reads were classified using a custom script, the order of reads predetermined by nature of the chemistry.

Reads were mapped to CHM13 v1.1 with addition of alternative sub-telomeric assemblies with minimap aligner allowing anchoring of reads from sub-telomeric regions into P and Q arms.

The mapped reads must have telomeric and sub-telomeric alignment. At least 20% of the read is aligned to the reference to be considered in future steps.

When a read has multiple mapping alignments, a single alignment is chosen by selecting the longest read coverage and identity.

Methylation was called with the Remora tool (Oxford Nanopore Technologies); https://github.com/nanoporetech/remora.

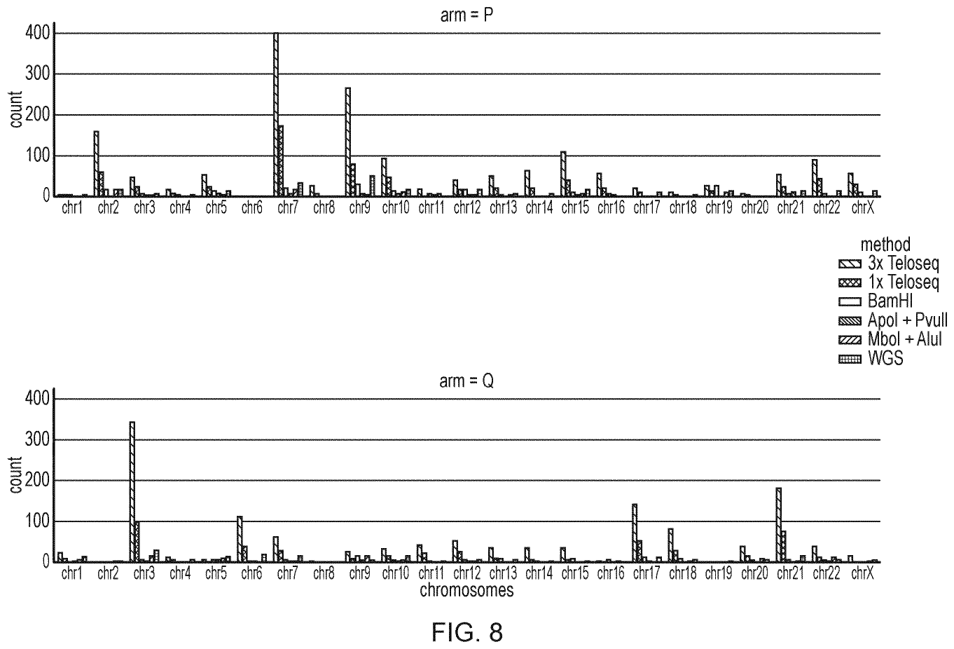

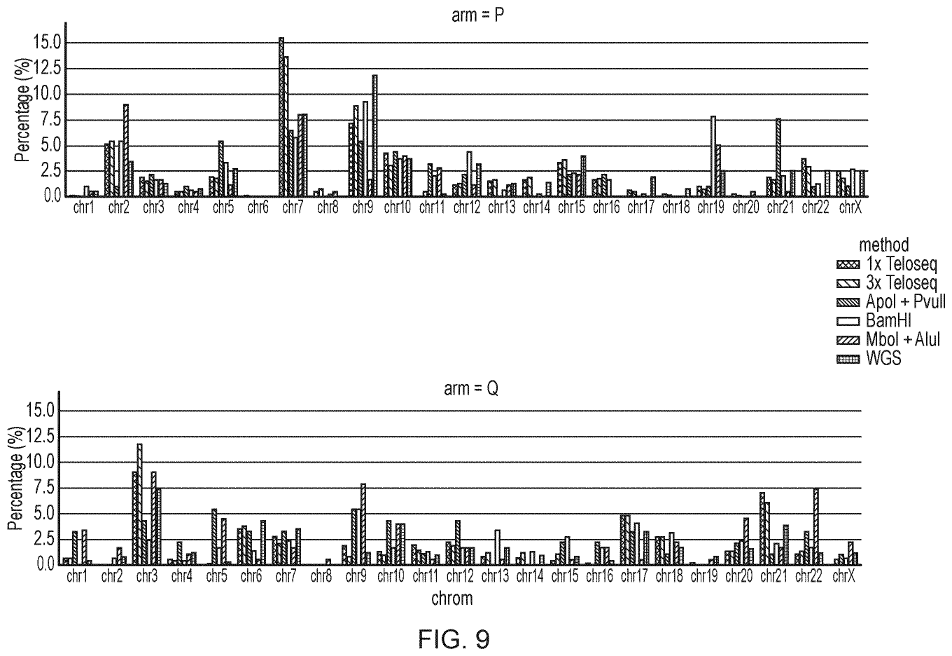

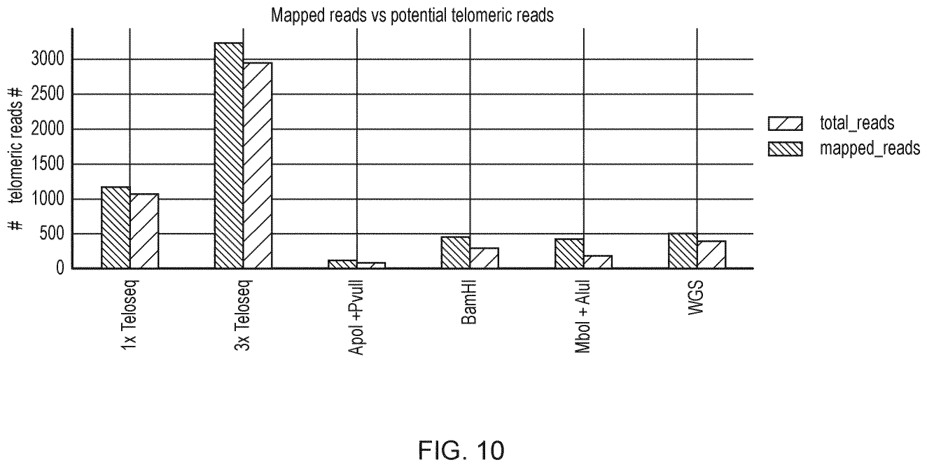

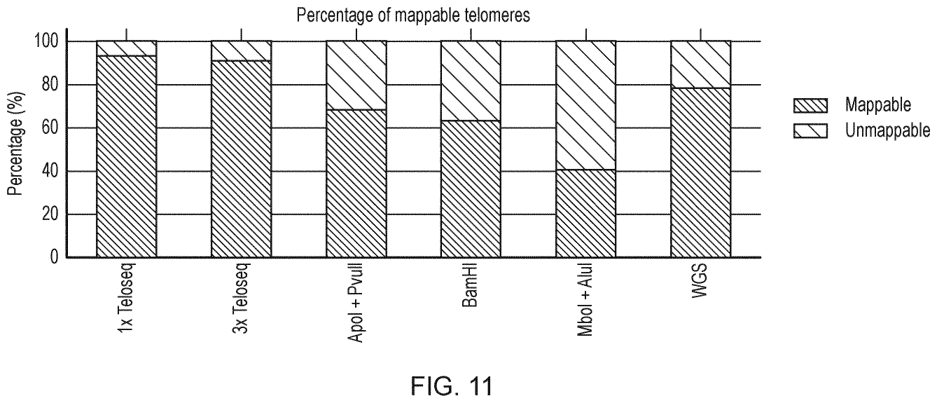

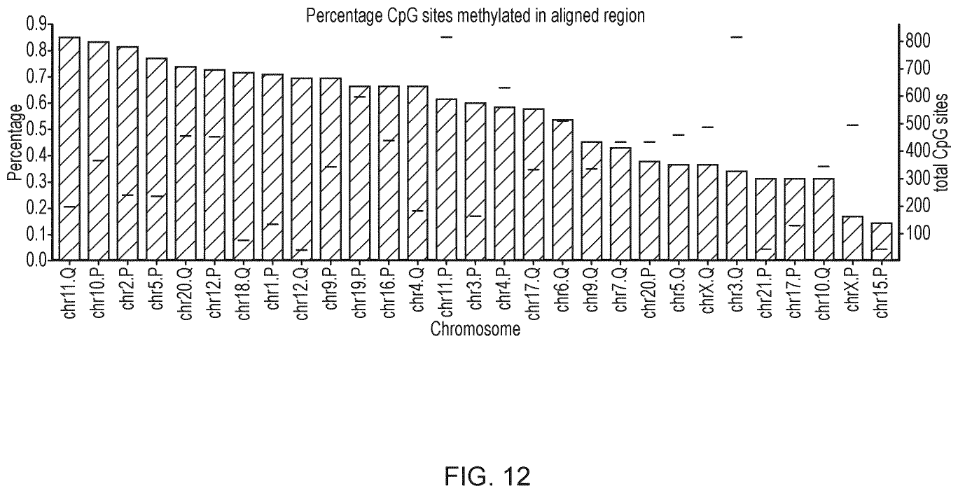

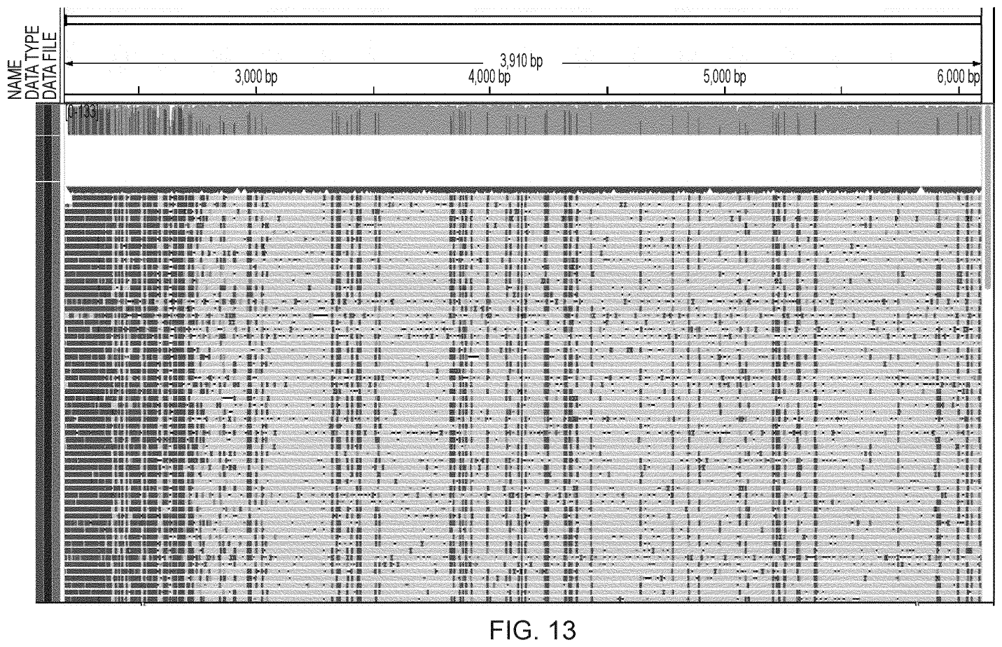

Results are shown in FIGS. 8-13.

### Example 5

Table 13 below shows the total number of telomeric reads, as well as the length of the subtelomere which is important for mapping reads uniquely, obtained with a number of different methods including restriction digestion methods and the “Teloseq” methods described herein. MobI+AluI has the shortest sub-telomeric lengths making mapping reads uniquely challenging and this is evident by the percentage mapped.

TABLE 13

| | | | Percent- | Avg | Avg sub- | Avg |
|---|---|---|---|---|---|---|
| | Total | Mapped | age | telomere | telomere | read |
| | telomeric | telomeric | mapped | length | length | len |
| Method | reads | reads | (%) | (Kb) | (Kb) | (Kb) |
| 3x | 3,239 | 2,936 | 90.6 | 4.31 | 12.50 | 16.81 |
| Teloseq | | | | | | |
| 1x | 1,169 | 1,086 | 92.2 | 4.34 | 9.75 | 14.09 |
| Teloseq | | | | | | |
| BamHI | 461 | 292 | 63.3 | 2.72 | 6.90 | 9.62 |
| MboI + | 425 | 176 | 41.4 | 1.99 | 0.53 | 2.53 |
| AluI | | | | | | |
| ApoI + | 135 | 92 | 63.3 | 2.79 | 1.43 | 4.23 |
| PvuII | | | | | | |
| WGS | 505 | 394 | 78.0 | 3.37 | 6.60 | 9.98 |

### Example 6

This Example demonstrates the use of a customised base-caller to identify and map telomere sequences enriched using the methods of the invention.

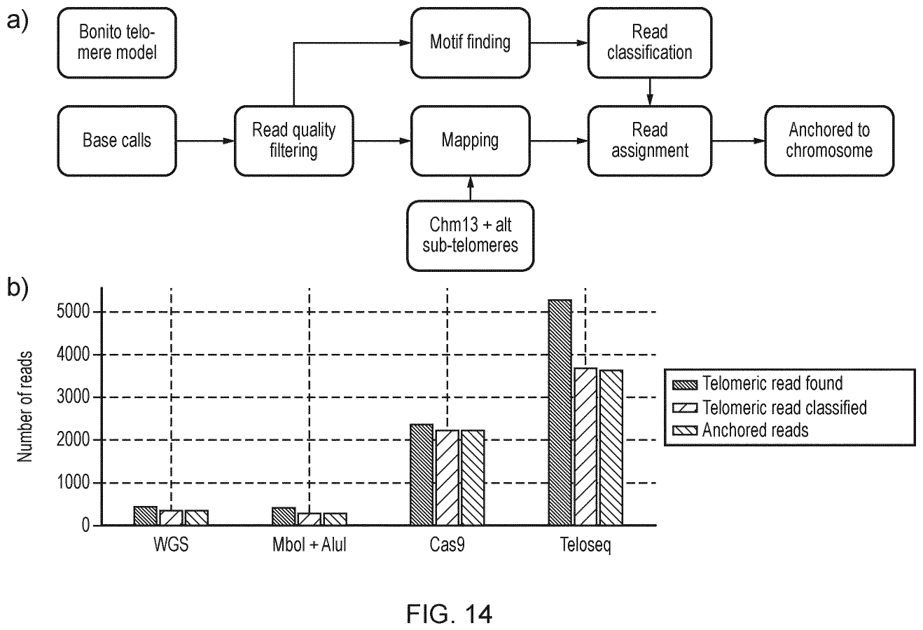

A Bonito base-calling model was trained to be specifically optimised for telomeric repeats and reads filtered using a noise-cancelling repeat finder (FIG. 14a). The isolated telomeric reads were aligned using minimap2 to the CHM13 v2.0 reference and alternative sub-telomeric assemblies. Alignments to the sub-telomeric regions were used to anchor a read to specific chromosome arms. Multiple telomere-enrichment methods were compared using the human cell line HG002 (FIG. 14b). The “Teloseq” method of the invention was compared to other approaches, including a restriction digestion with AluI and MboI, which do not cut in the telomeres, Cas9-based sequencing using guide RNAs targeted to the sub-telomeric region, and whole genome sequencing. FIG. 14b demonstrates that the “Teloseq” method of the invention enabled the identification of the greatest number of telomere sequences.

### Example 7

Telomere sequencing was carried out using techniques described above, with sequencing data obtained using R10 flow cells from Oxford Nanopore Technologies.

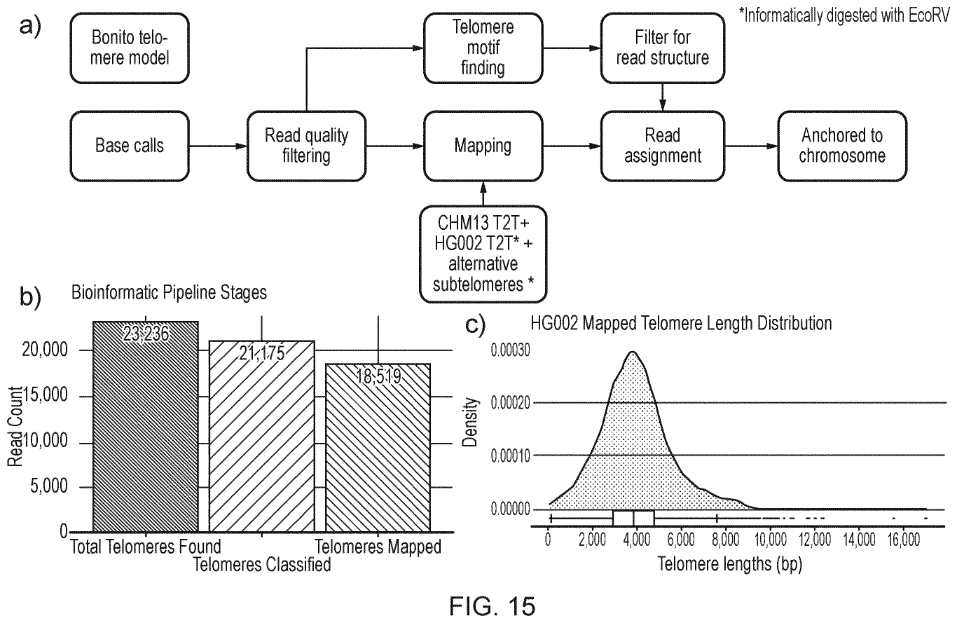

A Bonito base-calling model was trained to be specifically optimised for telomeric repeats, with stringent filtering stages used to isolate telomeric motifs. The isolated telomeric reads were aligned using minimap2 to a custom reference that includes: CHM13 v2.0, HG002, and alternative sub-telomeric assemblies that were virtually digested and P- and Q-arms retained (FIG. 15a).

Using the human cell line HG002, 23,236 reads were captured that contained the telomeric motif with at least 50 bps of continuous repeats, of which 18,519 telomeric reads uniquely mapped to chromosome arms (FIG. 15b). The mapped telomeric reads demonstrated a median telomere length of 3,826 bp (FIG. 15c).

### Example 8

This Example demonstrates phasing of telomeric reads, which can provide valuable telomere variant information.

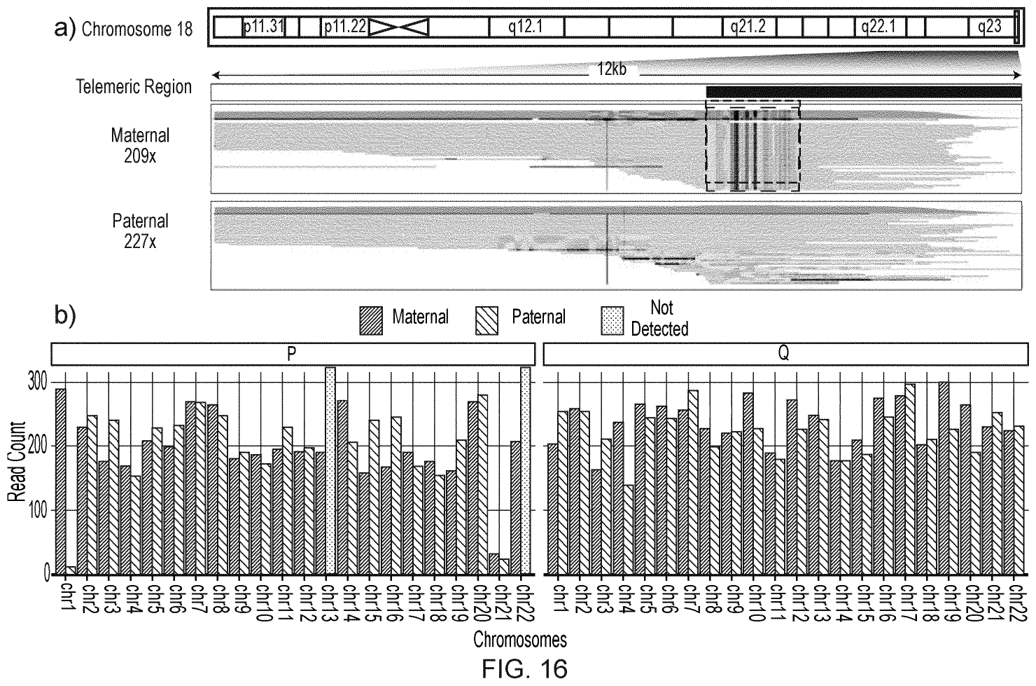

HG002 telomeric reads were uniquely mapped to a phased HG002 assembly of 88 autosomal, haplotyped chromosome arms, with successful alignments to 86 arms. In chromosome 18.q, telomeric SNP variants were observed in only the maternal copy, highlighted in dark grey (FIG. 16a). Each haplotyped chromosome arm has approximately 200× coverage when applying a mapping quality threshold of 30 and above (FIG. 16b). Lower-quality mapping was observed for certain chromosomes, such that of 1.p and 21.p. Pale boxes denote the paternal haplotyped chromosomes where no uniquely mapping telomere alignments to the reference were identified (chr13, chr22).

### Example 9

This Example demonstrates mapping of haplotyped telomere reads to provide high resolution of telomere length measurements.

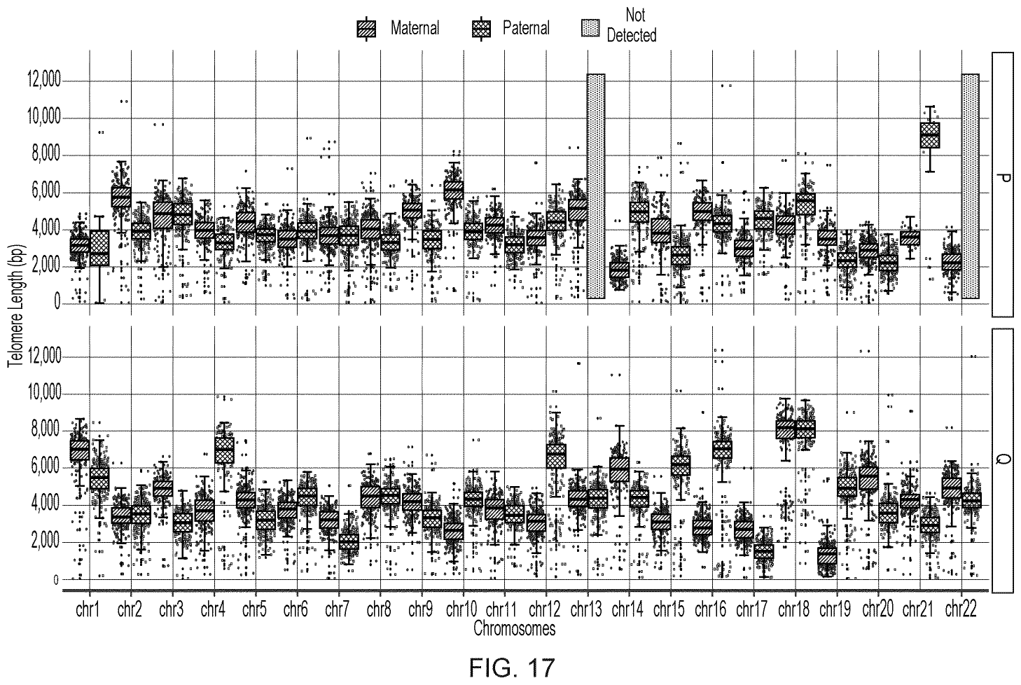

HG002 telomeric reads were uniquely mapped to a phased HG002 assembly to assess telomeric lengths (FIG. 17). Without phasing, the P-arm telomere length average is 3,768 bp, while the Q-arm telomere length average is 4,062 bp. On average, the delta between the maternal and paternal telomere lengths is 91. However, when the haplotypes are split by chromosome arms, there is a significant difference between the paternal haploid arms (Δ523). The paternal P-arm average is 3,690 bp, while the paternal Q-arm average is 4,213 bp. There is no significant difference between the maternal haploid arm telomere lengths (Δ73). The maternal P-arm average is 3,850 bp, while the maternal Q-arm average is 3,923 bp.

### Example 10

Example 1 was repeated using the telomere adaptors shown in Table 14 and the splint polynucleotide in Table 15.

TABLE 14
Telomere adaptors used in Example 10:

| Sequences | SEQ ID NO: |
|---|---|
| /5′phos/AGCAATACGTAACTGAACGAAGTCCCTAACCCTAACCCTAA 3′ | 14 |
| /5′phos/AGCAATACGTAACTGAACGAAGTTAACCCTAACCCTAACCC 3′ | 15 |
| /5′phos/AGCAATACGTAACTGAACGAAGTCTAACCCTAACCCTAACC 3′ | 16 |
| /5′phos/AGCAATACGTAACTGAACGAAGTCCTAACCCTAACCCTAAC 3′ | 17 |
| /5′phos/AGCAATACGTAACTGAACGAAGTAACCCTAACCCTAACCCT 3′ | 18 |
| /5′phos/AGCAATACGTAACTGAACGAAGTACCCTAACCCTAACCCTA 3′ | 19 |

TABLE 15
Splint polynucleotide used in Example 10:

| Splint | Sequences | SEQ ID NO: |
|---|---|---|
| S3 | ACTTCGTTCGGTGACTTGAGGACAGCAAT | 20 |

1. A method for characterising at least part of a telomere, the method comprising:
(a) ligating a polynucleotide telomere adaptor to the 5′ end of the non-overhanging strand at the end of the telomere wherein the 3′ end of the adaptor specifically hybridises to the first part of the overhanging strand and the 5′ end of the adaptor does not hybridise to the opposite part of the overhanging strand; and
(b) using the telomere adaptor to characterise the ligated, non-overhanging strand of the at least part of the telomere in the 5′ to 3′ direction from the end of the telomere.

2. A method according to claim 1, wherein characterising the at least part of the telomere comprises (a) sequencing the at least part of the telomere, (b) measuring the length of the at least part of the telomere, (c) telomere to telomere assembly of a chromosome or genome, (d) identifying telomere or chromosome fusions, (e) identifying one or more modifications in the at least part of the telomere, (f) identifying the at least part of the telomere as a variant or (g) linking the at least part of the telomere to a particular cell or tissue type.

3. A method according to claim 1 or 2, wherein the method is for characterising (i) all of the telomere, (ii) all of the telomere and at least part of, or all of, the subtelomere, (iii) all of the telomere, all of the subtelomere and at least part of, or all of, the chromatin, (iv) all of the telomere, all of the subtelomere, all of the chromatin and at least part of, or all of, the opposite subtelomere, or (v) all of the telomere, all of the subtelomere, all of the chromatin, all of the opposite subtelomere and at least part of, or all of, the opposite telomere.

4. A method according any one of the preceding claims, wherein the method is for characterising at least part of one or both telomeres on each of two or more different chromosomes.

5. A method according to any one of the preceding claims, wherein step (a) further comprises hybridising a splint polynucleotide to the 5′ end of the telomere adaptor.

6. A method according to claim 5, wherein the splint polynucleotide is compatible with a sequencing adaptor.

7. A method according to any one of the preceding claims, wherein step (a) further comprises attaching a sequencing adaptor to the telomere adaptor and, if present, the splint polynucleotide, and step (b) comprises using the sequencing adaptor to characterise the ligated, non-overhanging strand of the at least part of the telomere in the 5′ to 3′ direction.

8. A method according to any one of claims 1-4, wherein step (a) further comprises ligating or covalently attaching a polynucleotide extension to the telomere adaptor or step (a) uses a telomere adaptor further comprising a polynucleotide extension at its 5′ end.

9. A method according to claim 8, wherein the polynucleotide extension comprises a sequencing adaptor or step (a) further comprises covalently attaching a sequencing adaptor to the polynucleotide extension and step (b) comprises using the sequencing adaptor to characterise the ligated, non-overhanging strand of the at least part of the telomere in the 5′ to 3′ direction from the end of the telomere.

10. A method according to claim 8 or 9, wherein the polynucleotide extension is covalently attached to the telomere adaptor or to the sequencing adaptor using click chemistry.

11. A method according to any one of the preceding claims, wherein the telomere adaptor comprises biotin.

12. A method according to claim 11, wherein step (a) further comprises using the biotin to enrich the ligated, non-overhanging strand of the at least part of the telomere.

13. A method according to any one of the preceding claims, wherein the 3′ end of the telomere adaptor is at least 5 or at least about 7 nucleotides in length.

14. A method according to any one of the preceding claims, wherein one or more linkers or a spacers are present between the 3′ end of the telomere adaptor and the 5′ end of the telomere adaptor.

15. A method according to claim 14, wherein the linker is a flexible linker or spacer.

16. A method according to any one of the preceding claims, wherein the method comprises before step (a) contacting the telomere with a population of six telomere adaptors each of which has a 3′ end which specifically hybridises to one of the six possible sequences of the first part of the overhanging strand and a 5′ end which does not hybridise to the opposite part of the overhanging strand.

17. A method according to any one of the preceding claims, wherein the ligated, non-overhanging strand is characterised using a nanopore.

18. A method according to any one of the preceding claims, wherein the method does not comprise (i) restriction digestion and/or (ii) amplifying the at least part of a telomere or polymerase chain reaction (PCR).

19. A method according to any one of the preceding claims, wherein the method further comprises characterising the non-ligated, overhanging strand of the at least part of the telomere in the 5′ to 3′ direction to the end of the telomere.

20. A method according to claim 19, wherein the method comprises using a polymer-guided effector protein to create a double stranded break at the opposite end of the at least part of the telomere from the telomere end and attaching a sequencing adaptor to the opposite end and using the sequencing adaptor to characterise the non-ligated, overhanging strand of the at least part of the telomere in the 5′ to 3′ direction to the end of the telomere.

21. A method according to any one of claims 1-18, wherein the method is repeated at the other end of a chromosome and the method comprises characterising both strands of the whole chromosome.

22. A method for characterising at least part of a telomere, the method comprising (a) ligating a polynucleotide telomere adaptor to the 5′ end of the non-overhanging strand at the end of the telomere wherein the 3′ end of the specifically hybridises to the first part of the overhanging strand and the 5′ end of the adaptor does not hybridise to the opposite part of the overhanging strand, (b) using a polymer-guided effector protein to create a double stranded break at the opposite end of the at least part of the telomere from the telomere end and attaching a sequencing adaptor to the opposite end and (c) using the telomere adaptor to characterise the ligated non-overhanging strand of at least part of the telomere in the 5′ to 3′ direction from the end of the telomere and using the sequencing adaptor to characterise the non-ligated overhanging strand of the at least part of the telomere in the 5′ to 3′ direction to the end of the telomere.

23. A polynucleotide telomere adaptor wherein the 3′ end of the adaptor specifically hybridises to the first part of the overhanging strand at the end of a telomere and the 5′ end of the adaptor does not hybridise to the opposite part of the overhanging strand at the end of the telomere.

24. A polynucleotide telomere adaptor according to claim 23, wherein the adaptor is as defined in claim 11, 13 or 14.

25. A population of six telomere adaptors each of which has a 3′ end which specifically hybridises to one of the six possible sequences of the first part of the overhanging strand at the end of a telomere and a 5′ end which does not hybridise to the opposite part of the overhanging strand at the end of the telomere.

26. A population according to claim 25, wherein each adaptor is as defined in claim 11, 13 or 14.

27. A kit for characterising at least part of a telomere, comprising (a) one or more polynucleotide telomere adaptors according to claim 23 or 24 or a population of six telomere adaptors according to claim 25 or 26 and (b) one or more splint polynucleotides or one or more polynucleotide extensions.

28. A kit according to claim 27, wherein the telomere adaptor(s) and the one or more polynucleotide extensions comprise click chemistry groups.

29. A kit according to claim 27 or 28, wherein the kit further comprises one or more sequencing adaptors.

30. A kit according to any one of claims 27-29, wherein the kit further comprises a polymer-guided effector protein and one or more guide polymers.

31. A system comprising (a) one or more polynucleotide telomere adaptors according to claim 23 or 24 or a population of six telomere adaptors according to claim 25 or 26 and (b) a nanopore.
